(19) United States
(12) Patent Application Publication
(10) Pub. No.: US 2004/0220148 A1
(43) Pub. Date: Nov. 4, 2004

(54) 5-Membered ring heterocycles as inhibitors of leucocyte adhesion and as VLA-4 antagonists

(75) Inventors: Hans Ulrich Stilz, Frankfurt am Main (DE); Volkman Wehner, Sandberg (DE); Jochen Knolle, Kriftel (DE); Eckart Bartnik, Wiesbaden-Delkenheim (DE); Christoph Huels, Wackernheim (DE)

Correspondence Address: Patricia Granados, Heller Ehrman White & McAuliffe LLP, 1666 K Street, N.W., Suite 300, Washington, DC, 20006, US

(73) Assignee: Aventis Pharma Deutschland GmbH, Frankfurt am Main (DE)

(21) Appl. No.: 10/691,630

(22) Filed: Oct. 24, 2003

Related U.S. Application Data
(63) Continuation of application No. 08/971,960, filed on Nov. 17, 1997, now abandoned.

(30) Foreign Application Priority Data
Nov. 15, 1996 (DE) 196 47 380.2

(51) Int. Cl. A61K 31/675; A61K 31/513; A61K 31/426; A61K 31/421

(52) U.S. Cl. 514/80; 514/269; 514/369; 514/376; 514/389

(57) ABSTRACT

Compounds of the formula I

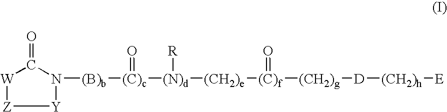

in which B, D, E, R, W, Y, Z, b, c, d, e, f, g and h have the meanings indicated in the claims, are inhibitors of the adhesion and migration of leucocytes and/or antagonists of the adhesion receptor VLA-4 which belongs to the group of integrins. The invention relates to the use of compounds of the formula I and of pharmaceutical preparations which contain such compounds for the treatment and prophylaxis of diseases which are caused by an undesired extent of leucocyte adhesion and/or leucocyte migration or which are associated therewith or in which cell-cell or cell-matrix interactions which are based on interactions of VLA-4 receptors with their ligands play a part, for example of inflammatory processes, of rheumatoid arthritis or of allergic disorders, and it also relates to the use of compounds of the formula I for the production of pharmaceuticals for use in such diseases. It further relates to novel compounds of the formula I.

The present invention relates to compounds of the formula I

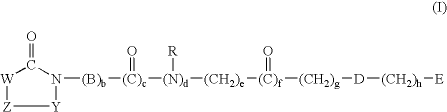

as inhibitors of the adhesion and migration of leucocytes and/or as antagonists of the adhesion receptor VLA-4 which belongs to the group of integrins. The invention relates to the use of compounds of the formula I and of pharmaceutical preparations which contain such compounds for the treatment or prophylaxis of diseases which are caused by an undesired extent of leucocyte adhesion and/or leucocyte migration or which are associated therewith or in which cell-cell or cell-matrix interactions which are based on interactions of VLA-4 receptors with their ligands play a part, for example of inflammatory processes, of rheumatoid arthritis or of allergic disorders, and it also relates to the use of compounds of the formula I for the production of pharmaceuticals for use in such diseases. It further relates to novel compounds of the formula I.

The integrins are a group of adhesion receptors which play an important part in cell-cell-binding and cell-extracellular matrix-binding processes. They have an αβ-heterodimeric structure and exhibit a wide cellular distribution and a high extent of evolutive conservation. The integrins include, for example, the fibrinogen receptor on platelets, which interacts especially with the RGD sequence of fibrinogen, or the vitronectin receptor on osteoclasts, which interacts especially with the RGD sequence of vitronectin or of osteopontin. The integrins are divided into three major groups, the β2 subfamily with the representatives LFA-1, Mac-1 and p150/95, which are responsible in particular for cell-cell interactions of the immune system, and the subfamilies β1 and β3, whose representatives mainly mediate cell adhesion to components of the extracellular matrix (Ruoslahti, Annu. Rev. Biochem. 1988, 57, 375). The integrins of the β1 subfamily, also called VLA proteins (very late (activation) antigen), include at least six receptors which interact specifically with fibronectin, collagen and/or laminin as ligands. Within the VLA family, the integrin VLA-4 (α4β1) is a typical in so far as it is mainly restricted to lymphoid and myeloid cells and is responsible in these for cell-cell interactions with a large number of other cells. For example, VLA-4 mediates the interaction of T and B lymphocytes with the heparin II-binding fragment of human plasma fibronectin (FN). The binding of VLA-4 with the heparin II-binding fragment of plasma fibronectin is especially based on an interaction with an LDVP sequence. In contrast to the fibrinogen or vitronectin receptor, VLA-4 is not a typical RGD-binding integrin (Kilger and Holzmann, J. Mol. Meth. 1995, 73, 347).

The leucocytes circulating in the blood normally exhibit only a low affinity for the vascular endothelial cells which line the blood vessels. Cytokines which are released from inflamed tissue cause the activation of endothelial cells and thus the expression of a large number of cell surface antigens. These include, for example, the adhesion molecules ELAM-1 (endothelial cell adhesion molecule-1; also designated as E-selectin), which, inter alia, binds neutrophiles, ICAM-1 (intercellular adhesion molecule-1), which interacts with LFA-I (leucocyte function-associated antigen 1) on leucocytes, and VCAM-1 (vascular cell adhesion molecule-1), which binds various leucocytes, inter alia lymphocytes (Osborn et al., Cell 1989, 59, 1203). VCAM-1, like ICAM-1, is a member of the immunoglobulin gene superfamily. VCAM-1 (first known as INCAM-110) was identified as an adhesion molecule which is induced on endothelial cells by inflammatory cytokines such as TNF and IL-1 and lipopolysaccharides (LPS). Elices et al. (Cell 1990, 60, 577) showed that VLA-4 and VCAM-1 form a receptor-ligand pair which mediates the adhesion of lymphocytes to activated endothelium. The binding of VCAM-1 to VLA-4 does not take place here due to an interaction of the VLA-4 with an RGD sequence; such one is not contained in VCAM-1 (Bergelson et al., Current Biology 1995, 5, 615). VLA-4, however, also occurs on other leucocytes, and the adhesion of leucocytes other than lymphocytes is also mediated via the VCAM-1/VLA-4 adhesion mechanism. VLA-4 thus represents an individual example of a β1 integrin receptor which, via the ligands VCAM-1 and fibronectin, plays an important part in cell-cell interactions and in cell-extracellular matrix interactions.

The cytokine-induced adhesion molecules play an important part in the recruitment of leucocytes into extravascular tissue regions. Leucocytes are recruited into inflammatory tissue regions by cell adhesion molecules which are expressed on the surface of endothelial cells and serve as ligands for leucocyte cell surface proteins or protein complexes (receptors) (the terms ligand and receptor can also be used vice versa). Leucocytes from the blood must first adhere to endothelial cells before they can migrate into the synovium. Since VCAM-1 binds to cells which carry the integrin VLA-4 (α4β1), such as eosinophils, T and B lymphocytes, monocytes or also neutrophils, it and the VCAM-1/VLA-4 mechanism have the function of recruiting cells of this type from the blood stream into areas of infection and inflammatory foci (Elices et al., Cell 1990, 60, 577; Osborn, Cell 1990, 62, 3; Issekutz et al., J. Exp. Med. 1996, 183, 2175).

The VCAM-1/VLA-4 adhesion mechanism has been connected with a number of physiological and pathological processes. Apart from cytokine-induced endothelium, VCAM-1 is additionally expressed, inter alia, by the following cells: myoblasts, lymphoid dendritic cells and tissue macrophages, rheumatoid synovium, cytokine-stimulated neural cells, parietal epithelial cells of the Bowman's capsule, the renal tubular epithelium, inflamed tissue during heart and kidney transplant rejection and by intestinal tissue in graft-versus-host disease. VCAM-1 is also found to be expressed on those tissue areas of the arterial endothelium which correspond to early arteriosclerotic plaques of a rabbit model. Additionally, VCAM-1 is expressed on follicular dendritic cells of human lymph nodes and is found on stroma cells of the bone marrow, for example in the mouse. The latter finding points to a function of VCAM-1 in B-cell development. Apart from cells of hematopoietic origin, VLA-4 is also found, for example, on melanoma cell lines, and the VCAM-1/VLA-4 adhesion mechanism is connected with the metastasis of such tumors (Rice et al., Science 1989, 246, 1303).

The main form in which VCAM-1 occurs in vivo on endothelial cells and which is the dominant form in vivo is designated as VCAM-7D and carries seven immunoglobulin domains. The domains 4, 5 and 6 are similar in their amino acid sequences to the domains 1, 2 and 3. The fourth domain is removed in a further form, consisting of six domains, designated here as VCAM-6D, by alternative splicing. VCAM-6D can also bind VLA-4-expressing cells.

Further details on VLA-4, VCAM-1, integrins and adhesion proteins are found, for example, in the articles by Kilger and Holzmann, J. Mol. Meth. 1995, 73, 347; Elices, Cell Adhesion in Human Disease, Wiley, Chichester 1995, p. 79; Kuijpers, Springer Semin. Immunopathol. 1995, 16, 379.

On account of the role of the VCAM-1/VLA-4 mechanism in cell adhesion processes which are of importance, for example, in infections, inflammations or atherosclerosis, it has been attempted by means of interventions into these adhesion processes to control diseases, in particular, for example, inflammations (Osborn et al., Cell 1989, 59, 1203). A method of doing this is the use of monoclonal antibodies which are directed against VLA-4. Monoclonal antibodies (mAB) of this type which as VLA-4 antagonists block the interaction between VCAM-1 and VLA-4, are known. Thus, for example, the anti-VLA-4 mAB HP2/1 and HP1/3 inhibit the adhesion of VLA-4-expressing Ramos cells (B-cell-like cells) to human umbilical cord endothelial cells and to VCAM-1-transfected COS cells.

The anti-VCAM-1 mAB 4B9 likewise inhibits the adhesion of Ramos cells, Jurkat cells (T-cell-like cells) and HL60 cells (granulocyte-like cells) to COS cells transfected with genetic constructs which cause VCAM-6D and VCAM-7D to be expressed. In vitro data with antibodies which are directed against the α4 subunit of VLA-4 show that the adhesion of lymphocytes to synovial endothelial cells is blocked, an adhesion which plays a part in rheumatoid arthritis (van Dinther-Janssen et al., J. Immunol. 1991, 147, 4207).

In vivo experiments have shown that an experimental autoimmune encephalomyelitis can be inhibited by anti-α4 mAB. The migration of leucocytes into an inflammatory focus is likewise blocked by a monoclonal antibody against the α4 chain of VLA-4. The influencing of the VLA-4-dependent adhesion mechanism by antibodies was also investigated in an asthma model in order to investigate the role of VLA-4 in the recruitment of leucocytes in inflamed lung tissue (U.S. Ser. No. 07/821,768; EP-A-626 861). The administration of anti-VLA-4 antibodies inhibited the late-phase reaction and respiratory tract overreaction in allergic sheep.

The VLA-4-dependent cell adhesion mechanism was also investigated in a primate model of inflammatory bowel disease (IBD). In this model, which corresponds to ulcerative colitis in man, the administration of anti-VLA-4 antibodies resulted in a significant reduction in the acute inflammation.

Moreover, it was possible to show that VLA-4-dependent cell adhesion plays a part in the following clinical conditions including the following chronic inflammatory processes: rheumatoid arthritis (Cronstein and Weismann, Arthritis Rheum. 1993, 36, 147; Elices et al., J. Clin. Invest. 1994, 93, 405), diabetes mellitus (Yang et al., Proc. Natl. Acad. Sci. USA 1993, 90, 10494), systemic lupus erythematosus (Takeuchi et al., J. Clin. Invest. 1993, 92, 3008), allergies of the delayed type (type IV allergy) (Elices et al., Clin. Exp. Rheumatol. 1993, 11, p.77), multiple sclerosis (Yednock et al., Nature 1992, 356, 63), malaria (Ockenhouse et al., J. Exp. Med. 1992, 176, 1183), arteriosclerosis (Obrien et al., J. Clin. Invest. 1993, 92, 945), transplantation (Isobe et al., Transplantation Proceedings 1994, 26, 867-868), various malignancies, for example melanoma (Renkonen et al., Am. J. Pathol. 1992, 140, 763), lymphoma (Freedman et al., Blood 1992, 79, 206) and others (Albelda et al., J. Cell Biol. 1991, 114, 1059).

VLA-4 blocking by suitable antagonists accordingly offers effective therapeutic possibilities, in particular, for example, of treating various inflammatory conditions including asthma and IBD. The particular relevance of VLA-4 antagonists for the treatment of rheumatoid arthritis in this respect results, as already stated, from the fact that leucocytes from the blood must first adhere to endothelial cells before they can migrate into the synovium, and that the VLA-4 receptor plays a part in this adhesion. The fact that VCAM-1 is induced by inflammatory agents on endothelial cells (Osborn, Cell 1990, 62, 3; Stoolman, Cell 1989, 56, 907), and the recruitment of various leucocytes into areas of infection and inflammatory foci has already been dealt with above. In this respect, T cells adhere to activated endothelium mainly via the LFA-1/ICAM-1 and VLA-4/VCAM-1 adhesion mechanisms (Springer, Cell 1994,76, 301). On most synovial T cells, the binding capacity of VLA-4 for VCAM-1 is increased in rheumatoid arthritis (Postigo et al., J. Clin. Invest. 1992, 89, 1445). Additionally, an increased adhesion of synovial T cells to fibronectin has been observed (Laffon et al., J. Clin. Invest. 1991, 88, 546; Morales-Ducret et al., J. Immunol. 1992, 149, 1424). VLA-4 is also upregulated both in the course of its expression and with respect to its function on T lymphocytes of the rheumatoid synovial membrane. The blocking of the binding of VLA-4 to its physiological ligands VCAM-1 and fibronectin makes possible an effective prevention or alleviation of articular inflammatory processes. This is also confirmed by experiments with the antibody HP2/1 on Lewis rats with adjuvant arthritis, in which an effective prevention of illness has been observed (Barbadillo et al., Springer Semin. Immunopathol. 1995, 16, 427). VLA-4 is thus an important therapeutic target molecule.

The abovementioned VLA-4 antibodies and the use of antibodies as VLA-4 antagonists are described in the Patent Applications WO-A-93/13798, WO-A-93/15764, WO-A-94116094, WO-A-94/17828 and WO-A-95/19790. In the Patent Applications WO-A-94/15958, WO-A-95/15973, WO-A-96/00581, WO-A-96/06108 and WO-A-96/20216, peptide compounds are described as VLA-4 antagonists. The use of antibodies and peptide compounds as pharmaceuticals, however, is afflicted with disadvantages, for example lack of oral availability, easy degradability or immunogenic action on longer-term use, and there is thus a need for VLA-4 antagonists having a favorable profile of properties for use in therapy and prophylaxis.

WO-A-94/21607 and WO-A-95/14008 describe substituted 5-membered ring heterocycles and EP-A-449 079, EP-A-530 505 (U.S. Pat. No. 5,389,614), WO-A-93/18057, EP-A-566 919 (U.S. Pat. No. 5,397,796), EP-A-580 008 (U.S. Pat. No. 5,424,293) and EP-A-584 694 (U.S. Pat. No. 5,554,594) describe hydantoin derivatives which have platelet aggregation-inhibiting acitvity. There are, however, not found any indications of a VLA-4 antagonism of these compounds. Surprisingly, it has now been found that these compounds also inhibit leucocyte adhesion and are VLA-4 antagonists.

The present invention thus relates to the use of compounds of the formula I

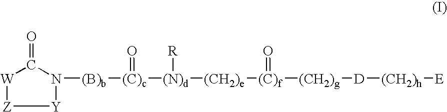

in which

W is R¹-A-C(R¹³) or R¹-A-CH═C;

Y is a carbonyl, thiocarbonyl or methylene group;

Z is N(R⁰), oxygen, sulfur or a methylene group;

A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, phenylene-(C₂-C₆)-alkenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent radical from the group consisting of (C₁-C₆)-alkylene, (C₂-C₆)-alkenylene, phenylene, phenylene-(C₁-C₃)-alkyl, (C₁-C₃)-alkylenephenyl, where the bivalent (C₁-C₆)-alkylene radical can be unsubstituted or substituted by a radical from the group consisting of (C₁-C₈)-alkyl, (C₂-C₈)-alkenyl, (C₂-C₈)-alkynyl, (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl and heteroaryl-(C₁-C₆)-alkyl optionally substituted in the heteroaryl radical;

D is C(R²)(R³), N(R³) or CH═C(R³);

E is tetrazolyl, (R⁸O)₂P(O), HOS(O)₂, R⁹NHS(O)₂ or R¹⁰CO;

R is hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R⁰ is hydrogen, (C₁-C₈)-alkyl, (C₃-C₁₂)-cycloalkyl, (C₃-C₁₂)-cycloalkyl-(C₁-C₈)-alkyl, (C₆-C₁₂)-bicycloalkyl, (C₆-C₁₂)-bicycloalkyl-(C₁-C₈)-alkyl, (C₆-C₁₂)-tricycloalkyl, (C₆-C₁₂)-tricycloalkyl-(C₁-C₈)₇alkyl, optionally substituted (C₆-C₁₄)-aryl, (C₆-C₁₄)-aryl-(C₁-C₈)-alkyl optionally substituted in the aryl radical, optionally substituted heteroaryl, heteroaryl-(C₁-C₈)-alkyl optionally substituted in the heteroaryl radical, CHO, (C₁-C₈)-alkyl-CO, (C₃-C₁₂)-cycloalkyl-CO, (C₃-C₁₂)-cycloalkyl-(C₁-C₈)-alkyl-CO, (C₆-C₁₂)-bicycloalkyl-CO, (C₆-C₁₂)-bicycloalkyl-(C₁-C₈)-alkyl-CO, (C₆-C₁₂)-tricycloalkyl-CO, (C₆-C₁₂)-tricycloalkyl-(C₁-C₈)-alkyl-CO, optionally substituted (C₆-C₁₄)-aryl-CO, (C₆-C₁₄)-aryl-(C₁-C₈)-alkyl-CO optionally substituted in the aryl radical, optionally substituted heteroaryl-CO, heteroaryl-(C₁-C₈)-alkyl-CO optionally substituted in the heteroaryl radical, (C₁-C₈)-alkyl-S(O)ₙ, (C₃-C₁₂)-cycloalkyl-S(O)ₙ, (C₃-C₁₂)-cycloalkyl-(C₁-C₈)-alkyl-S(O)ₙ, (C₆-C₁₂)-bicycloalkyl-S(O)ₙ, (C₆-C₁₂)-bicycloalkyl-(C₁-C₈)-alkyl-S(O)ₙ, (C₆-C₁₂)-tricycloalkyl-S(O)ₙ, (C₆-C₁₂)-tricycloalkyl-(C₁-C₈)-alkyl-S(O)ₙ, optionally substituted (C₆-C₁₄)-aryl-S(O)ₙ, (C₆-C₁₄)-aryl-(C₁-C₈)-alkyl-S(O)ₙ optionally substituted in the aryl radical, optionally substituted heteroaryl-S(O)ₙ or heteroaryl-(C₁-C₈)-alkyl-S(O)ₙ optionally substituted in the heteroaryl radical, where n is 1 or 2;

R¹ is X¹—NH—C(═NH)—(CH₂)ₚ or X¹—NH—(CH₂)ₚ, where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″), where R′ and R″ independently of one another have the meanings of X;

R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;

R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R¹⁴, CONHR¹⁴, CSNHR¹⁴, COOR¹⁵, CON(CH₃)R¹⁵ or CONHR¹⁵;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵,

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino and trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹³ is hydrogen, (C₁-C₆)-alkyl, (C₆-C₁₄)-aryl-(C₁-C₈)-alkyl optionally substituted in the aryl radical or (C₃-C₈)-cycloalkyl;

R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)-aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alalkylphenyl-(C₁-C₃)-alkylaminocarbonyl, (C₁-C₁₈)-alkylcarbonyl-amino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;

R¹⁵ is R¹⁶—(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which, can also be substituted by one or more identical or different substituents from the group consisting of (C₁-C₄)-alkyl and oxo;

b, c, d and f independently of one another are 0 or 1, but cannot all simultaneously be 0;

e, g and h independently of one another are 0, 1, 2, 3, 4, 5 or 6;

in all their stereoisomeric forms and mixtures thereof in any ratio, and of their physiologically tolerable salts for the production of pharmaceuticals for inhibition of the adhesion and/or migration of leucocytes or for inhibition of the VLA-4 receptor, i.e. of pharmaceuticals for the treatment or prophylaxis of diseases in which leucocyte adhesion and/or leucocyte migration has an undesired extent, or of diseases in which VLA-4-dependent adhesion processes play a part, for example of inflammatory disorders, and to the use of the compounds of the formula I in the treatment and prophylaxis of diseases of this type.

Alkyl radicals can be straight-chain or branched. This also applies if they carry substituents or occur as substituents of other radicals, for example in alkoxy, alkoxycarbonyl or aralkyl radicals. The same applies to alkylene radicals. Examples of suitable (C₁-C₂₈)-alkyl radicals are: methyl, ethyl, propyl, butyl, pentyl, hexyl, heptyl, octyl, decyl, undecyl, dodecyl, tridecyl, pentadecyl, hexadecyl, heptadecyl, nonadecyl, eicosyl, docosyl, tricosyl, pentacosyl, hexacosyl, heptacosyl, octacosyl, isopropyl, isobutyl, isopentyl, neopentyl, isohexyl, 3-methylpentyl, 2,3,5-trimethylhexyl, sec-butyl, tert-butyl, tert-pentyl. Preferred alkyl radicals are methyl, ethyl, propyl, isopropyl, butyl, isobutyl, sec-butyl and tert-butyl. Examples of alkylene radicals are methylene, ethylene, tri-, tetra-, penta- and hexamethylene or methyl ene substituted by an alkyl radical, for example methylene which is substituted by a methyl group, an ethyl group, an isopropyl group, an isobutyl group or a tert-butyl group.

Alkenyl and alkenylene radicals as well as alkynyl radicals can also be straight-chain or branched. Examples of alkenyl radicals are vinyl, 1-propenyl, 2-propenyl(=allyl), butenyl, 3-methyl-2-butenyl, examples of alkenylene radicals are vinylene or propenylene and examples of alkynyl radicals are ethynyl, 1-propynyl or 2-propynyl(=propargyl).

Cycloalkyl radicals are, in particular, cyclopropyl, cyclobutyl, cyclopentyl, cyclohexyl, cycloheptyl, cyclooctyl, cyclononyl, cyclodecyl, cycloundecyl and cyclododecyl, but which can also be substituted by, for example, (C₁-C₄)-alkyl. Examples of substituted cycloalkyl radicals which may be mentioned are 4-methylcyclohexyl and 2,3-dimethylcyclopentyl. The same applies to cycloalkylene radicals.

The 6- to 24-membered bicyclic and tricyclic radicals R¹⁶ are formally obtained by abstraction of a hydrogen atom from bicyclic systems or tricyclic systems. The bicyclic systems and tricyclic systems on which they are based can contain only carbon atoms as ring members, i.e. they can be bicycloalkanes or tricycloalkanes, but they can also contain one to four identical or different heteroatoms from the group consisting of nitrogen, oxygen and sulfur, i.e. they can be aza-, oxa- and thiabicyclo- and -tricycloalkanes. If heteroatoms are contained, preferably one or two heteroatoms, in particular nitrogen or oxygen atoms, are contained. The heteroatoms can assume any desired positions in the bi- or tricyclic structure; they can be located in the bridges, or in the case of nitrogen atoms, also at the bridgeheads. Both the bicyclo- and tricycloalkanes and their heterocyclic analogs can be completely saturated or can contain one or more double bonds. They preferably contain one or two double bonds or, in particular, are completely saturated. Both the bicyclo- and tricycloalkanes and the heterocyclic analogs and both the saturated and the unsaturated representatives can be unsubstituted or substituted in any desired suitable positions by one or more oxo groups and/or one or more identical or different (C₁-C₄)-alkyl groups, for example methyl or isopropyl groups, preferably methyl groups. The free bond of the bi- or tricyclic radical can be located in any desired position of the molecule; the radical can thus be bonded via a bridgehead atom or an atom in a bridge. The free bond can also be located in any desired stereochemical position, for example in an exo- or an endo position.

Examples of parent structures of bicyclic ring systems from which a bicyclic radical can be derived are norbornane(=bicyclo[2.2.1]heptane), bicyclo[2.2.2]octane and bicyclo[3.2.1]octane, examples of unsaturated or substituted systems or systems containing heteroatoms are 7-azabicyclo[2.2.1]-heptane, bicyclo[2.2.2]oct-5-ene and camphor(=1,7,7-trimethyl-2-oxobicyclo[2.2.1]heptane).

Examples, of systems from which a tricyclic radical can be derived are twistane(=tricyclo[4.4.0.0^{3,8}]decane), adamantane(=tricyclo[3.3.1.1^{3,7}]-decane), noradamantane(=tricyclo[3.3.1.0^{3,7}]nonane), tricyclo[2.2.1.0^{2,6}]heptane, tricyclo[5.3.2.0^{4,9}]dodecatne, tricyclo[5.4.0.0^{2,9}]undecane or tricyclo[5.5.0^{3,11}]tridecane.

Preferably, bicyclic or tricyclic radicals are derived from bridged bicyclic systems or tricyclic systems, i.e. from systems in which rings together have two or more than two atoms. Bicyclic or tricyclic radicals having 6 to 18 ring members are additionally preferred, particularly preferably those having 7 to 12 ring members.

Specifically particularly preferred bi- and tricyclic radicals are the 2-norbornyl radical, both that with the free bond in the exo position and also that with the free bond in the endo position, the 2-bicyclo[3.2.1]octyl radical, the 1-adamantyl radical, the 2-adamantyl radical and the noradamantyl radical, for example the 3-noradamantyl radical. The 1- and the 2-adamantyl radicals are moreover preferred.

(C₆-C₁₄)-aryl groups are, for example, phenyl, naphthyl, biphenylyl, anthryl or fluorenyl, where 1-naphthyl, 2-naphthyl and in particular phenyl are preferred. Aryl radicals, in particular phenyl radicals, can be mono- or polysubstituted, preferably mono- di- or trisubstituted, by identical or different radicals from the group consisting of (C₁-C₈)-alkyl, in particular (C₁-C₄)-alkyl, (C₁-C₈)-alkoxy, in particular (C₁-C₄)-alkoxy, halogen, nitro, amino, trifluoromethyl, hydroxyl, methylenedioxy, ethylenedioxy, cyano, hydroxycarbonyl, aminocarbonyl, (C₁-C₄)-alkoxycarbonyl, phenyl, phenoxy, benzyl, benzyloxy, (R⁸O)₂P(O), (R⁸O)₂P(O)—O—, tetrazolyl. The same applies, for example, to radicals such as aralkyl or arylcarbonyl. Aralkyl radicals are, in particular, benzyl as well as 1- and 2-naphthylmethyl, 2-, 3- and 4-biphenylylmethyl and 9-fluorenylmethyl which can also be substituted. Substituted aralkyl radicals are, for example, benzyl and naphthylmethyl substituted in the aryl moiety by one or more (C₁-C₈)-alkyl radicals, in particular (C₁-C₄)-alkyl radicals, for example 2-, 3- and 4-methylbenzyl, 4-isobutylbenzyl, 4-tert-butylbenzyl, 4-octylbenzyl, 3,5-dimethylbenzyl, pentamethylbenzyl, 2-, 3-, 4-, 5-, 6-, 7- and 8-methyl-1-naphthylmethyl, 1-, 3-, 4-, 5-, 6-, 7- and 8-methyl-2-naphthylmethyl, or benzyl or naphthylmethyl substituted in the aryl moiety by one or more (C₁-C₈)-alkoxy radicals, in particular (C₁-C₄)-alkoxy radicals, for example 4-methoxybenzyl, 4-neopentyloxybenzyl, 3,5-dimethoxybenzyl, 3,4-methylenedioxybenzyl, 2,3,4-trimethoxybenzyl, further 2-, 3- and 4-nitrobenzyl, halobenzyl, for example 2-, 3- and 4-chloro- and 2-, 3- and 4-fluorobenzyl, 3,4-dichlorobenzyl, pentafluorobenzyl, trifluoromethylbenzyl, for example 3- and 4-trifluoromethylbenzyl or 3,5-bis(trifluoromethyl)benzyl. Substituted aralkyl radicals, however, can also have different substituents. Examples of pyridyl are 2-pyridyl, 3-pyridyl and 4-pyridyl.

In monosubstituted phenyl radicals, the substituent can be located in the 2-, the 3- or the 4-position, the 3- and the 4-positions being preferred. If phenyl is disubstituted, the substituents can be in the 1,2-, 1,3- or 1,4-position relative to one another. Disubstituted phenyl can thus be substituted in the 2,3-position, the 2,4-position, the 2,5-position, the 2,6-position, the 3,4-position or the 3,5-position, relative to the linkage site. Preferably, in disubstituted phenyl radicals the two substituents are arranged in the 3-position and the 4-position, relative to the linkage site. The same applies for phenylene radicals which, for example, can be present as 1,4-phenylene or as 1,3-phenylene.

Phenylene-(C₁-C₆)-alkyl is, in particular, phenylenemethyl and phenyleneethyl. Phenylene-(C₂-C₆)-alkenyl is, in particular, phenyleneethenyl and phenylenepropenyl.

Mono- or bicyclic 5- to 12-membered heterocyclic rings are, for example, pyrrolyl, furyl, thienyl, imidazolyl, pyrazolyl, oxazolyl, isoxazolyl, thiazolyl, isothiazolyl, tetrazolyl, pyridyl, pyrazinyl, pyrimidinyl, indolyl, isoindolyl, indazolyl, phthalazinyl, quinolyl, isoquinolyl, quinoxalinyl, quinazolinyl, cinnolinyl, or a benzo-fused, cyclopenta-, cyclohexa- or cyclohepta-fused derivative of these radicals.

If not stated otherwise, these heterocycles can be substituted on a nitrogen atom by (C₁-C₇)-alkyl, for example methyl or ethyl, phenyl or phenyl-(C₁-C₄)-alkyl, for example benzyl, and/or on one or more carbon atoms by (C₁-C₄)-alkyl, halogen, hydroxyl, (C₁-C₄)-alkoxy, for example methoxy, phenyl-(C₁-C₄)-alkoxy, for example benzyloxy, or oxo and can be aromatic or partially or completely saturated. Nitrogen heterocycles can also be present as N-oxides.

The radicals of aromatic heterocycles, i.e. heteroaryl radicals, preferably contain a 5-membered ring heterocycle or 6-membered ring heterocycle having one, two, three or four, in particular one or two, identical or different heteroatoms from the group consisting of nitrogen, oxygen and sulfur, which can also be fused, for example benzo-fused, and which can be substituted by one or more, for example one, two, three or four, identical or different substituents. Suitable substituents are, for example, (C₁-C₈)-alkyl, in particular (C₁-C₄)-alkyl, (C₁-C₈)-alkoxy, in particular (C₁-C₄)-alkoxy, halogen, nitro, amino, trifluoromethyl, hydroxyl, methylenedioxy, ethylenedioxy, cyano, hydroxycarbonyl, aminocarbonyl, (C₁-C₄)-alkoxycarbonyl, phenyl, phenoxy, benzyl, benzyloxy, (R⁸O)₂P(O), (R⁸O)₂P(O)—O— or tetrazolyl.

Examples of heterocyclic radicals are 2- or 3-pyrrolyl, phenylpyrrolyl, for example 4- or 5-phenyl-2-pyrrolyl, 2-furyl, 3-furyl, 2-thienyl, 3-thienyl, 4-imidazolyl, methylimidazolyl, for example 1-methyl-2-, -4- or -5-imidazolyl, 1,3-thiazol-2-yl, 2-pyridyl, 3-pyridyl, 4-pyridyl, 2-, 3- or 4-pyridyl-N-oxide, 2-pyrazinyl, 2-, 4- or 5-pyrimidinyl, 2-, 3- or 5-indolyl, substituted 2-indolyl, for example 1-methyl-, 5-methyl-, 5-methoxy-, 5-benzyloxy-, 5-chloro- or 4,5-dimethyl-2-indolyl, 1-benzyl-2- or -3-indolyl, 4,5,6,7-tetrahydro-2-indolyl, cyclohepta[b]-5-pyrrolyl, 2-, 3- or 4-quinolyl, 1-, 3- or 4-isoquinolyl, 1-oxo-1,2-dihydro-3-isoquinolyl, 2-quinoxalinyl, 2-benzofuranyl, 2-benzothienyl, 2-benzoxazolyl or benzothiazolyl. Partially hydrogenated or completely hydrogenated heterocyclic rings are, for example, dihydropyridinyl, pyrrolidinyl, for example 2-, 3- or 4-(N-methylpyrrolidinyl), piperazinyl, morpholinyl, thiomoipholinyl, tetrahydrothienyl, benzodioxolanyl.

Halogen is fluorine, chlorine, bromine or iodine, in particular fluorine or chlorine.

If chiral, natural or unnatural amino acids can be present in the D- or L-form. α-Amino acids are preferred. Examples which may be mentioned are (cf. Houben-Weyl, Methoden der organischen Chemie [Methods of organic chemistry], Volume 15/1 and 15/2, Georg Thieme Verlag, Stuttgart, 1974):

Aad, Abu, γAbu, ABz, 2ABz, εAca, Ach, Acp, Adpd, Ahb, Aib, βAib, Ala, βAla, ΔAla, Alg, All, Ama, Amt, Ape, Apm, Apr, Arg, Asn, Asp, Asu, Aze, Azi, Bai, Bph, Can, Cit, Cys, (Cys)₂, Cyta, Daad, Dab, Dadd, Dap, Dapm, Dasu, Djen, Dpa, Dtc, Fel, Gln, Glu, Gly, Guv, hAla, hArg, hCys, hGln, hGlu, His, hIle, hLeu, hLys, hMet, hPhe, hPro, hSer, hThr, hTrp, hTyr, Hyl, Hyp, 3Hyp, Ile, Ise, Iva, Kyn, Lant, Lcn, Leu, Lsg, Lys, βLys, ΔLys, Met, Mim, Min, nArg, Nle, Nva, Oly, Orn, Pan, Pec, Pen, Phe, Phg, Plc, Pro, ΔPro, Pse, Pya, Pyr, Pza, Qin, Ros, Sar, Sec, Sem, Ser, Thi, βThi, Thr, Thy, Thx, Tia, Tle, Tly, Trp, Trta, Tyr, Val, tert-butylglycine (Tbg), neopentylglycine (Npg), cyclohexylglycine (Chg), cyclohexylalanine (Cha), 2-thienylalanine (Thia), 2,2-diphenylaminoacetic acid, 2-(p-tolyl)-2-phenylaminoacetic acid, 2-(p-chlorophenyl)-amino acetic acid.

Amino acid side chains are understood as meaning side chains of natural or unnatural amino acids. Azamino acids are natural or unnatural amino acids in which the central unit

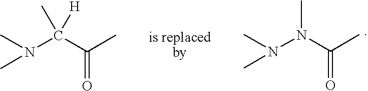

Suitable radicals of an imino acid are, in particular, radicals of heterocycles from the following group: pyrrolidine-2-carboxylic acid; piperidine-2-carboxylic acid; 1,2,3,4-tetrahydroisoquinoline-3-carboxylic acid; decahydroisoquinoline-3-carboxylic acid; octahydroindole-2-carboxylic acid; decahydroquinoline-2-carboxylic acid; octahydrocyclopenta[b]pyrrole-2-carboxylic acid; 2-azabicyclo[2.2.2]octane-3-carboxylic acid; 2-azabicyclo[2.2.1]heptane-3-carboxylic acid; 2-azabicyclo[3.1.0]hexane-3-carboxylic acid; 2-azaspiro[4.4]nonane-3-carboxylic acid; 2-azaspiro[4.5]decane-3-carboxylic acid; spiro(bicyclo[2.2.1]heptane)-2,3-pyrrolidine-5-carboxylic acid; spiro(bicyclo[2.2.2]octane)-2,3-pyrrolidine-5-carboxylic acid; 2-azatricyclo[4.3.0.1^{6,9}]decane-3-carboxylic acid; decahydrocyclohepta[b]pyrrole-2-carboxylic acid; decahydrocycloocta[c]pyrrole-2-carboxylic acid; octahydrocyclopenta[c]pyrrole-2-carboxylic acid; octahydroisoindole-1-carboxylic acid; 2,3,3a,4,6a-hexahydrocyclopenta[b]pyrrole-2-carboxylic acid; 2,3,3a,4,5,7a-hexahydroindole-2-carboxylic acid; tetrahydrothiazole-4-carboxylic acid; isoxazolidine-3-carboxylic acid; pyrazolidine-3-carboxylic acid, hydroxypyrrolidine-2-carboxylic acid, all of which can optionally be substituted (see following formulae):

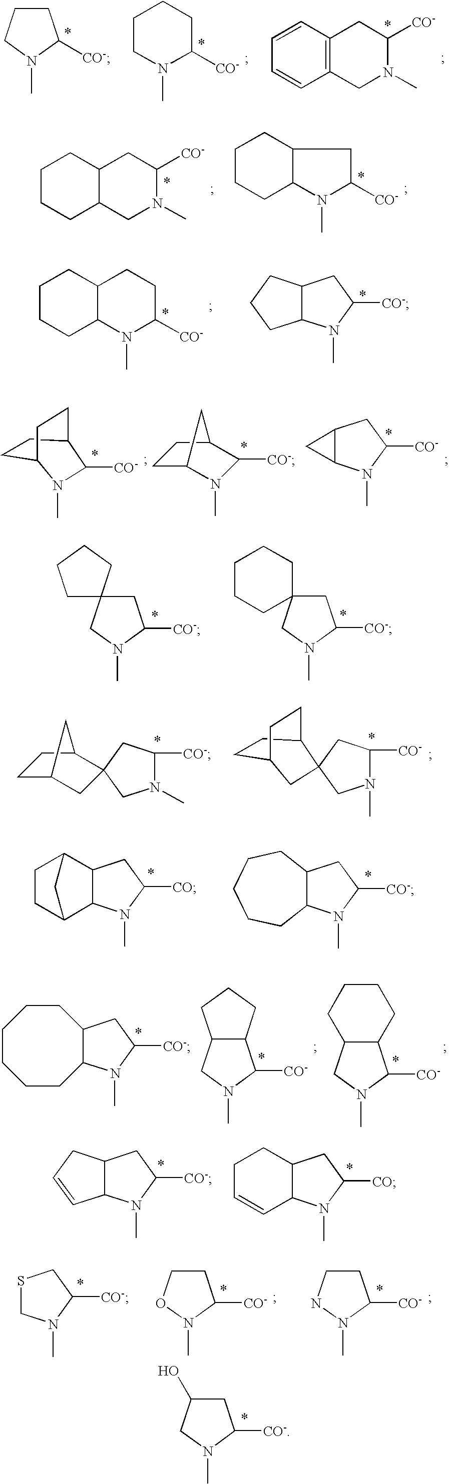

The heterocycles on which the abovementioned radicals are based are disclosed, for example, in U.S. Pat. No. 4,344,949; U.S. Pat. No. 4,374,847; U.S. Pat. No. 4,350,704; EP-A 29,488; EP-A 31,741; EP-A 46,953; EP-A 49,605; EP-A 49,658; EP-A 50,800; EP-A 51,020; EP-A 52,870; EP-A 79,022; EP-A 84,164; EP-A 89,637; EP-A 90,341; EP-A 90,362; EP-A 105,102; EP-A 109,020; EP-A 111,873; EP-A 271,865 and EP-A 344,682.

Dipeptides can contain natural or unnatural amino acids, imino acids as well as azamino acids as structural units. The natural or unnatural amino acids, imino acids, azamino acids and dipeptides can further be present also as esters or amides, such as, for example, as the methyl ester, ethyl ester, isopropyl ester, isobutyl ester, tert-butyl ester, benzyl ester, unsubstituted amide, ethylamide, semicarbazide or ω-amino-(C₂-C₈)-alkylamide.

Functional groups of the amino acids, imino acids and dipeptides can be present in protected form. Suitable protective groups such as, for example, urethane protective groups, carboxyl protective groups and side chain protective groups are described in Hubbuch, Kontakte (Merck) 1979, No. 3, pages 14 to 23, and in Bullesbach, Kontakte (Merck) 1980, No. 1, pages 23 to 35. The following may be mentioned in particular: Aloc, Pyoc, Fmoc, Tcboc, Z, Boc, Ddz, Bpoc, Adoc, Msc, Moc, Z(NO₂), Z(Halₙ), Bobz, Iboc, Adpoc, Mboc, Acm, tert-butyl, OBzl, ONbzl, OMbzl, Bzl, Mob, Pic, Trt.

Physiologically tolerable salts of the compounds of the formula I are, in particular, pharmaceutically utilizable or nontoxic salts.

Such salts are formed, for example, from compounds of the formula I which contain acidic groups, for example carboxyl, with alkali metals or alkaline earth metals, such as, for example, Na, K, Mg and Ca, and also with physiologically tolerable organic amines, such as, for example, triethylamine, ethanolamine or tris(2-hydroxyethyl)amine.

Compounds of the formula I which contain basic groups, for example an amino group, an amidino group or a guanidino group, form salts with inorganic acids, such as, for example, hydrochloric acid, sulfuric acid or phosphoric acid, and with organic carboxylic or sulfonic acids, such as, for example, acetic acid, citric acid, benzoic acid, maleic acid, fumaric acid, tartaric acid, methanesulfonic acid or p-toluenesulfonic acid.

Salts can be obtained from the compounds of the formula I by customary methods known to the person skilled in the art, for example by combination with an organic or inorganic acid or base in a solvent or dispersant, or alternatively from other salts by anion exchange or cation exchange.

The compounds of the formula I can be present in stereoisomeric forms. If the compounds of the formula I contain one or more centers of asymmetry, these can independently of one another have the S configuration or the R configuration. The invention includes all possible stereoisomers, for example enantiomers and diastereomers, and mixtures of two or more stereoisomeric forms, for example mixtures of enantiomers and/or diastereomers, in all ratios. The invention thus relates to enantiomers in enantiomerically pure form, both as levo- and as dextrorotatory antipodes, in the form of racemates and in the form of mixtures of the two enantiomers in all ratios. If cis/trans isomerism is present, the invention relates to both the cis form and the trans form and mixtures of these forms.

The compounds of the formula I according to the invention can moreover contain mobile hydrogen atoms, i.e. can be present in various tautomeric forms. The present invention also relates to all these tautomers. The present invention furthermore includes all solvates of compounds of the formula I, for example hydrates or adducts with alcohols, as well as derivatives of the compounds of the formula I, for example esters, prodrugs and metabolites which act like the compounds of the formula I.

The individual structural elements in the formula I preferably have the following meanings.

W is preferably R¹-A-C(R¹³).

A is preferably methylene, ethylene, trimethylene, tetramethylene, pentamethylene, cyclohexylene, phenylene, phenylenemethyl or phenyleneethyl.

Y is preferably a carbonyl group.

Z is preferably N(R⁰).

B is preferably methylene, ethylene, trimethylene, tetramethylene, vinylene, phenylene or substituted methylene or ethylene. B is particularly preferably a bivalent methylene radical or ethylene radical (=1,2-ethylene), where each of these radicals can be unsubstituted or substituted, in particular an unsubstituted or substituted methylene radical. These two radicals are very particularly preferably substituted. If a bivalent methylene radical or ethylene radical (=1,2-ethylene) representing B is substituted, it is preferably substituted by a radical from the group consisting of (C₁-C₈)-alkyl, (C₂-C₆)-alkenyl, (C₂-C₆)-alkynyl, (C₃-C₈)-cycloalkyl, in particular (C₅-C₆)-cycloalkyl, (C₃-C₈)-cycloalkyl-(C₁-C₄)-alkyl, in particular (C₅-C₆)-cycloalkyl-(C₁-C₄)-alkyl, optionally substituted (C₆-C₁₀)-aryl, (C₆-C₁₀)-aryl-(C₁-C₄)-alkyl optionally substituted in the aryl radical, optionally substituted heteroaryl or heteroaryl-(C₁-C₄)-alkyl optionally substituted in the heteroaryl radical, and it is particularly preferably substituted by (C₁-C₈)-alkyl, i.e. by a straight-chain or branched alkyl radical having 1, 2, 3, 4, 5, 6, 7 or 8 carbon atoms.

D is preferably C(R²)(R³).

E is preferably R¹⁰CO.

R is preferably hydrogen, (C₁-C₆)-alkyl or benzyl, in particular hydrogen, methyl or ethyl.

R⁰ is preferably (C₁-C₈)-alkyl, (C₃-C₈)-cycloalkyl, (C₃-C₈)-cycloalkyl-(C₁-C₄)-alkyl, optionally substituted (C₆-C₁₄)-aryl or (C₆-C₁₄)-aryl-(C₁-C₈)-alkyl optionally substituted in the aryl radical. R⁰ is particularly preferably (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical, very particularly preferably optionally substituted (C₆-C₁₄)-aryl or (C₆-C₁₄)-aryl-(C₁-C₈)-alkyl optionally substituted in the aryl radical, moreover preferably (C₆-C₁₄)-aryl-(C₁-C₄)-alkyl optionally substituted in the aryl radical. It is specifically preferred if R⁰ is biphenylylmethyl, naphthylmethyl or benzyl, each of which is unsubstituted or mono- or polysubstituted in the aryl radical.

R¹ is preferably X—NH—C(═NH), X—NH—(C═NX)—NH or X—NH—CH₂.

X and X¹ are preferably hydrogen, (C₁-C₆)-alkylcarbonyl, (C₁-C₆)-alkoxycarbonyl, (C₁-C₁₈)-alkylcarbonyloxy-(C₁-C₆)-alkoxycarbonyl or (C₆-C₁₄)-aryl-(C₁-C₆)-alkoxycarbonyl, hydroxyl, X¹ is additionally R′—NH—C(═N—R″), where R′ and R″ independently of one another have the preferred meanings of X.

R² is preferably hydrogen or (C₁-C₈)-alkyl.

R³ is preferably (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, pyridyl, R¹¹NH, R⁴CO, COOR⁴, CON(CH₃)R¹⁴, CONHR¹⁴, CSNHR¹⁴, COOR¹⁵, CON(CH₃)R¹⁵ or CONHR¹⁵, particularly preferably optionally substituted (C₆-C₁₄)-aryl, R¹¹NH, CON(CH₃)R¹⁴ or CONHR¹⁴.

R⁴ and R¹⁴ are preferably (C₁-C₈)-alkyl which can optionally be substituted as indicated in the definition of R⁴ or R¹⁴.

R¹³ is preferably hydrogen and in particular (C₁-C₆)-alkyl, (C₃-C₈)-cycloalkyl or benzyl, where a very particularly preferred alkyl radical representing R¹³ is the methyl radical.

R¹⁵ is preferably R¹⁶—(C₁-C₃)-alkyl or R¹⁶, particularly preferably R¹⁶—(C₁)-alkyl or R¹⁶. Moreover, when R³ is COOR¹⁵, R¹⁵ is preferably the exo-2-norbornyl radical, the endo-2-norbornyl radical or the bicyclo[3.2.1]octyl radical, and when R³ is CONHR¹⁵, R¹⁵ is the exo-2-norbornyl radical, the endo-2-norbornyl radical, the 3-noradamantyl radical and in particular the 1-adamantyl radical, the 2-adamantyl radical, the 1-adamantylmethyl radical or the 2-adamantylmethyl radical.

R¹⁶ is preferably a 7- to 12-membered bridged bicyclic or tricyclic radical, which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo.

b, c and d independently of one another are 1.

e, g and h preferably independently of one another are the numbers 0, 1, 2 or 3.

Compounds preferred for the use according to the invention are those in which, in the formula I simultaneously

W is R¹-A-C(R¹³) or R¹-A-CH═C;

Y is a carbonyl, thiocarbonyl or methylene group;

Z is N(R⁰), oxygen, sulfur or a methylene group;

A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, phenylene-(C₂-C₆)-alkenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent radical from the group consisting of (C₁-C₆)-alkylene, (C₂-C₆)-alkenylene, phenylene, phenylene-(C₁-C₃)-alkyl, (C₁-C₃)-alkylene-phenyl;

D is C(R²)(R³), N(R³) or CH═C(R³);

E is tetrazolyl, (R⁸O)₂P(O), HOS(O)₂, R⁹NHS(O)₂ or R¹⁰CO;

R and R⁰ independently of one another are hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is X—NH—C(═NH)—(CH₂)ₚ or X¹—NH—(CH₂)ₚ, where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″) where R′ and R″ independently of one another have the meanings of X;

R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;

R³ is hydrogen, (C₁-C₈)-alkyl, optionally substituted (C₆-C₁₄)-aryl, (C₆-C₁₄)-aryl-(C₁-C₈)-alkyl, optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R¹⁴, CONHR¹⁴, CSNHR¹⁴, COOR¹⁵, CON(CH₃)R¹⁵ or CONHR¹⁵;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)-aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino or trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxy carbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹³ is hydrogen, (C₁-C₆)-alkyl, (C₆-C₁₄)-aryl-(C₁-C₈)-alkyl optionally substituted in the aryl radical or (C₃-C₈)-cycloalkyl;

R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)-aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;

R¹⁵ is R¹⁶-(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo;

b, c, d and f independently of one another are 0 or 1, but cannot all simultaneously be 0;

e, g and h independently of one another are 0, 1, 2, 3, 4, 5 or 6;

in all their stereoisomeric forms and mixtures thereof in any ratio, and their physiologically tolerable salts.

Particularly preferred compounds of the formula I are those in which simultaneously

W is R¹-A-CH═C and therein A is a phenylene radical, or W is R¹-A-C(R¹³) and therein A is a bivalent radical from the group consisting of methylene, ethylene, trimethylene, tetramethylene, pentamethylene, cyclohexylene, phenylene, phenylenemethyl;

B is a bivalent radical from the group consisting of methylene, ethylene, trimethylene, tetramethylene, vinylene, phenylene, or is substituted methylene or ethylene;

E is R¹⁰CO;

R is hydrogen, (C₁-C₆)-alkyl or benzyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is X—NH—C(═NH), X—NH—C(═NX)—NH or X—NH—CH₂;

X is hydrogen, (C₁-C₆)-alkylcarbonyl, (C₁-C₆)-alkoxycarbonyl, (C₁-C₈)-alkylcarbonyloxy-(C₁-C₆)-alkoxycarbonyl, (C₆-C₁₄)-aryl-(C₁-C₆)-alkoxycarbonyl or hydroxyl;

R² is hydrogen or (C₁-C₈)-alkyl;

R³ is (C₁-C₈)-alkyl, optionally substituted (C₆-C₁₄)-aryl, (C₆-C₁₄)-aryl-(C₁-C₈)-alkyl, (C₃-C₈)-cycloalkyl, (C₂-C₈)-alkenyl; (C₂-C₈)-alkynyl, pyridyl, R¹¹NH, R⁴CO, COOR⁴, CONHR¹⁴, CSNHR⁴, COOR¹⁵ and CONHR¹⁵;

and e, g and h independently of one another are the numbers 0, 1, 2 or 3;

in all their stereoisomeric forms and mixtures thereof in any ratio, and their physiologically tolerable salts.

Very particularly preferred compounds of the formula I are those in which W is R¹-A-C(R¹³) and R¹³ is (C₁-C₆)-alkyl, (C₆-C₁₄)-aryl-(C₁-C₈)-alkyl optionally substituted in the aryl radical or (C₃-C₈)-cycloalkyl, in all their stereoisomeric forms and mixtures thereof in any ratio, and their physiologically tolerable salts.

A series of specifically preferred compounds of the formula I are those in which R³ is optionally substituted (C₆-C₁₄)-aryl, COOR⁴, R¹¹NH or CONHR¹⁴, where —NHR¹⁴ is the radical of an α-amino acid, its co-amino-(C₂-C₈)-alkylamide, its (C₁-C₈)-alkyl ester or its (C₆-C₁₄)-aryl-(C₁-C₄)-alkyl ester, in all their stereoisomeric forms and mixtures thereof in any ratio, and their physiologically tolerable salts. The radical of an α-amino acid —NHR¹⁴ is formally obtained by abstraction of a hydrogen atom from the amino group of the amino acid. It is specifically preferred in this series if R³ is CONHR¹⁴, where —NHR¹⁴ is the radical of the α-amino acids valine, lysine, phenylglycine, phenylalanine or tryptophan or their (C₁-C₈)-alkyl esters or (C₆-C₁₄)-aryl-(C₁-C₄)-alkyl esters.

Moreover preferred compounds of the formula I in this series are those in which simultaneously

W is R¹-A-C(R³);

Y is a carbonyl group;

Z is N(R⁰);

A is ethylene, trimethylene, tetramethylene, pentamethylene, cyclohexylene, phenylene or phenylenemethyl;

B is an unsubstituted or substituted methylene radical;

D is C(R²)(R³);

E is R¹⁰CO;

R is hydrogen or (C₁-C₄)-alkyl, in particular hydrogen, methyl or ethyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is H₂N—C(═NH), H₂N—C(═NH)—NH or H₂N—CH₂;

R² is hydrogen;

R³ is the radical CONHR¹⁴;

R¹⁰ is hydroxyl or (C₁-C₈)-alkoxy, preferably (C₁-C₄)-alkoxy;

R¹³ is (C₁-C₆)-alkyl, (C₃-C₇)-cycloalkyl or benzyl, in particular methyl;

R¹⁴ is methyl which is substituted by hydroxycarbonyl and a radical from the group consisting of (C₁-C₄)-alkyl, phenyl and benzyl, or is methyl which is substituted by (C₁-C₈)-alkoxycarbonyl, preferably (C₁-C₄)-alkoxycarbonyl, and a radical from the group consisting of (C₁-C₄)-alkyl, phenyl and benzyl;

b, c and d are 1 and e, f and g are 0;

h is 1 or 2; preferably 1;

in all their stereoisomeric forms and mixtures thereof in any ratio, and their physiologically tolerable salts.

If —NHR¹⁴ is a (C₁-C₈)-alkyl ester of an α-amino acid or R¹⁴ contains an alkoxycarbonyl radical, the methyl, ethyl, isopropyl, isobutyl or tert-butyl ester is preferred. If —NHR¹⁴ is a (C₆-C₁₄)-aryl-(C₁-C₄)-alkyl ester of an α-amino acid, the benzyl ester is preferred.

A further series of specifically preferred compounds of the formula I are those compounds in which simultaneously

W is R¹-A-CH═C and therein A is a phenylene radical, or W is R¹-A-C(R¹³) and therein A is a bivalent radical from the group consisting of methylene, ethylene, trimethylene, tetramethylene, pentamethylene, cyclohexylene, phenylene, phenylenemethyl;

B is a bivalent radical from the group consisting of methylene, ethylene, trimethylene, tetramethylene, vinylene, phenylene or is substituted methylene or ethylene;

E is R¹⁰CO;

R is hydrogen or (C₁-C₆)-alkyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is X—NH—C(═NH), X—NH—C(═NX)—NH or X—NH—CH₂;

X is hydrogen, (C₁-C₆)-alkylcarbonyl, (C₁-C₆)-alkoxycarbonyl, (C₁-C₈)-alkylcarbonyloxy-(C₁-C₆)-alkoxycarbonyl, (C₆-C₁₄)-aryl-(C₁-C₆)-alkoxycarbonyl or hydroxyl;

R² is hydrogen or (C₁-C₈)-alkyl;

R³ is CONHR¹⁵ or CONHR¹⁴ where R¹⁴ herein is a (C₁-C₈)-alkyl radical which is unsubstituted or substituted by one or more (C₆-C₁₄)-aryl radicals;

R¹⁵ is R¹⁶—(C₁-C₆)-alkyl or R¹⁶, where R¹⁶ is a 7- to 12-membered bridged bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo, and in particular R¹⁵ is an adamantyl radical or an adamantylmethyl radical;

and e, g and h independently of one another are the numbers 0, 1, 2 or 3 and b, c and d are 1;

in all their stereoisomeric forms and mixtures thereof in any ratio, and their physiologically tolerable salts.

Moreover preferred compounds of the formula I in this series are those in which simultaneously

W is R¹-A-C(R¹³);

Y is a carbonyl group;

Z is N(R⁰);

A is ethylene, trimethylene, tetramethylene, pentamethylene, cyclohexylene, phenylene or phenylenemethyl;

B is an unsubstituted or substituted methylene radical;

D is C(R²)(R³);

E is R¹⁰CO;

R is hydrogen or (C₁-C₄)-alkyl, in particular hydrogen, methyl or ethyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is H₂N—C(═NH), H₂N—C(═NH)—NH or H₂N—CH₂;

R² is hydrogen;

R³ is CONHR¹⁵ or CONHR¹⁴ where R¹⁴ herein is a (C₁-C₆)-alkyl radical which is unsubstituted or substituted by one or more (C₆-C₁₀)-aryl radicals;

R¹⁰ is hydroxyl or (C₁-C₈)-alkoxy, preferably (C₁-C₄)-alkoxy;

R¹³ is (C₁-C₆)-alkyl, (C₃-C₇)-cycloalkyl or benzyl, in particular methyl;

R¹⁵ is an adamantyl radical or an adamantylmethyl radical;

b, c and d are 1 and e, f and g are 0;

h is 1 or 2, preferably 1;

in all their stereoisomeric forms and mixtures thereof in any ratio, and their physiologically tolerable salts.

Furthermore, a series of specifically preferred compounds of the formula I are those in which simultaneously

W is R¹-A-C(R¹³);

Y is a carbonyl group;

Z is N(R⁰);

A is ethylene, trimethylene, tetramethylene, pentamethylene, cyclohexylene, phenylene, phenylenemethyl;

B is an unsubstituted or substituted methylene radical or ethylene radical; D is C(R²)(R³);

E is R¹⁰CO;

R is hydrogen or (C₁-C₄)-alkyl, in particular hydrogen, methyl or ethyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl which is optionally substituted in the aryl radical;

R¹ is H₂N—C(═NH), H₂N—C(═NH)—NH or H₂N—CH₂;

R² is hydrogen;

R³ is an unsubstituted phenyl radical or naphthyl radical, a phenyl radical or naphthyl radical substituted by one, two or three identical or different radicals from the group consisting of (C₁-C₄)-alkyl, (C₁-C₄)-alkoxy, hydroxyl, halogen, trifluoromethyl, nitro, methylenedioxy, ethylenedioxy, hydroxycarbonyl, (C₁-C₄)-alkoxycarbonyl, aminocarbonyl, cyano, phenyl, phenoxy, benzyl and benzyloxy, a pyridyl radical, a (C₁-C₄)-alkyl radical, a (C₂-C₄)-alkenyl radical, a (C₂-C₄)-alkynyl radical or a (C₅-C₆)-cycloalkyl radical, and in particular R³ is an unsubstituted or substituted phenyl radical or naphthyl radical;

R¹⁰ is hydroxyl or (C₁-C₈)-alkoxy, in particular (C₁-C₄)-alkoxy, and preferably R¹⁰ is a radical from the group consisting of hydroxyl, methoxy, ethoxy, propoxy and isopropoxy;

R¹³ is (C₁-C₆)-alkyl, (C₃-C₇)-cycloalkyl or benzyl, in particular methyl;

b, c and d are 1 and e, f and g are 0;

h is 1 or 2, preferably 1;

in all their stereoisomeric forms and mixtures thereof in any ratio, and their physiologically tolerable salts.

Finally, a series of specifically preferred compounds of the formula I are those in which simultaneously

W is R¹-A-C(R¹³);

Y is a carbonyl group;

Z is N(R⁰);

A is ethylene, trimethylene, tetramethylene, pentamethylene, cyclohexylene, phenylene, phenylenemethyl;

B is an unsubstituted or substituted methylene radical or ethylene radical;

D is C(R²)(R³);

E is R¹⁰CO;

R is hydrogen or (C₁-C₄)-alkyl, in particular hydrogen, methyl or ethyl;

R⁰ is (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is H₂N—C(═NH), H₂N—C(═NH)—NH or H₂N—CH₂;

R² is hydrogen;

R³ is R¹¹NH;

R¹⁰ is hydroxyl or (C₁-C₈)-alkoxy, in particular (C₁-C₄)-alkoxy, and preferably R¹⁰ is a radical from the group consisting of hydroxyl, methoxy, ethoxy, propoxy and isopropoxy;

R¹³ is (C₁-C₆)-alkyl, (C₃-C₇)-cycloalkyl or benzyl, in particular methyl;

b, c, d and e are 1 and f and g are 0;

h is 0;

in all their stereoisomeric forms and mixtures thereof in any ratio, and their physiologically tolerable salts.

Very specifically preferred compounds of the formula I are those in which a substituted methylene radical or substituted ethylene radical representing the group B carries as a substituent a radical from the group consisting of (C₁-C₈)-alkyl, (C₂-C₆)-alkenyl, (C₂-C₆)-alkynyl, (C₃-C₈)-cycloalkyl, in particular (C₅-C₆)-cycloalkyl, (C₃-C₈)-cycloalkyl-(C₁-C₄)-alkyl, in particular (C₅-C₆)-cycloalkyl-(C₁-C₄)-alkyl, optionally substituted (C₆-C₁₀)-aryl, (C₆-C₁₀)-aryl-(C₁-C₄)-alkyl optionally substituted in the aryl radical, optionally substituted heteroaryl and heteroaryl(C₁-C₄)-alkyl optionally substituted in the aryl radical, in all their stereoisomeric forms and mixtures thereof in any ratio, and their physiologically tolerable salts. Even more specifically preferred compounds of the formula I are those in which B is an unsubstituted methylene radical or a methylene radical which is substituted by a (C₁-C₈)-alkyl radical, in particular by a (C₁-C₆)-alkyl radical, in all their stereoisomeric forms and mixtures thereof in any ratio, and their physiologically tolerable salts.

Generally, compounds of the formula I are preferred which have a uniform configuration at chiral centers, e.g. at the chiral carbon atom representing D and at the center W in the 5-membered ring heterocycle in the formula I.

The compounds of the formula I can be prepared, for example, by fragment condensation of a compound of the formula II

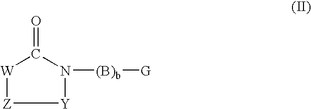

with a compound of the formula III,

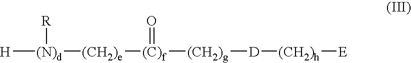

where W, Y, Z, B, D, E, R and b, d, e, f, g, and h are defined as indicated above and G is hydroxycarbonyl, (C₁-C₆)-alkoxycarbonyl, activated carboxylic acid derivatives, such as acid chlorides or active esters, or isocyanato.

For the condensation of the compounds of the formula II with those of the formula III, the coupling methods of peptide chemistry known per se (see, for example, Houben-Weyl, Methoden der Organischen Chemie, [Methods of organic chemistry], Volume 15/1 and 15/2, Georg Thieme Verlag, Stuttgart, 1974) are advantageously used. To do this, as a rule it is necessary that nonreacting amino groups present are protected by reversible protective groups during the condensation. The same applies to carboxyl groups not participating in the reaction, which are preferably present as (C₁-C₆)-alkyl, benzyl or tert-butyl esters. Amino group protection is unnecessary if the amino groups to be generated are still present as nitro or cyano groups and are formed, for example, by hydrogenation only after the coupling. After the coupling, the protective groups present are removed in a suitable manner. For example, NO₂ groups (guanidino protection), benzyloxycarbonyl groups and benzyl esters can be removed by hydrogenation. The protective groups of the tert-butyl type are removed under acidic conditions, while the 9-fluorenylmethyloxycarbonyl radical is removed by secondary amines. The compounds of the formula I can also be prepared, for example, by synthesizing the compounds stepwise on a solid phase according to customary methods, as is illustrated by way of example below.

Compounds of the formula II in which W is R¹-A-C(R¹³), Y is a carbonyl group and Z is NR⁰ can be prepared, for example, by first reacting compounds of the formula IV

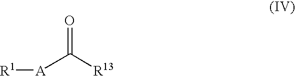

in a Bucherer reaction to give compounds of the formula V,

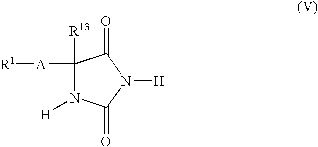

in which just as in the formula IV R¹, R¹³ and A are defined as indicated above (H. T. Bucherer, V. A. Lieb, J. Prakt. Chem. 141(1934), 5). Compounds of the formula VI,

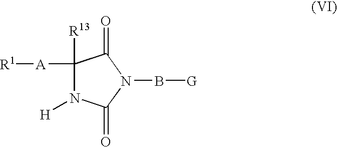

in which R¹, R¹³, A, B and G are defined as indicated above can then be obtained by first reacting the compounds of the formula V, for example, with an alkylating reagent which introduces the radical -B-G into the molecule. The reaction of compounds of the formula VI with a second reagent of the formula R⁰-LG, in which R⁰ has the meanings indicated above and LG is a nucleophilically substitutable leaving group, for example halogen, in particular chlorine or bromine, (C₁-C₄)-alkoxy, optionally substituted phenoxy or a heterocyclic leaving group such as, for example, imidazolyl, leads to the corresponding compounds of the formula II. These reactions can be carried out analogously to known methods familiar to the person skilled in the art. Depending on the individual case, it may be appropriate here, as in all steps in the synthesis of the compounds of the formula I, temporarily to block functional groups which could lead to side reactions or undesired reactions by means of a protective group strategy adapted to the synthesis problem, what is known to the person skilled in the art.

If W is R¹-A-CH═C, this structural element can be introduced, for example, by condensing an aldehyde with a 5-membered ring heterocycle which contains a methylene group in the position corresponding to the group W analogously to known methods.

Compounds of the formula I in which the 5-membered ring heterocycle is a dioxo- or thioxo-oxo-substituted imidazolidine ring in which W is R¹-A-C(R¹³), can also be obtained as follows:

By reaction of α-amino acids or N-substituted α-amino acids or preferably their esters, for example the methyl, ethyl, tert-butyl or benzyl esters, for example of a compound of the formula VII,

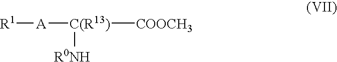

in which R⁰, R¹, R¹³ and A are defined as indicated above, with an isocyanate or isothiocyanate, for example, of the formula VIII,

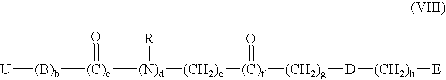

in which B, D, E and R and also b, c, d, e, f, g and h are defined as indicated above and U is isocyanato or isothiocyanato, there are obtained urea or thiourea derivatives, for example of the formula IX

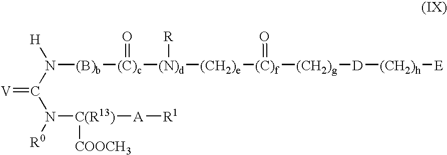

for which the definitions indicated above apply and in which V is oxygen or sulfur, and which by heating with acid are cyclized with hydrolysis of the ester functions to give compounds of the formula Ia

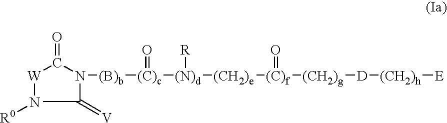

in which V is oxygen or sulfur, W is R¹-A-C(R¹³) and for which otherwise the meanings indicated above apply. The cyclization of the compounds of the formula IX to the compounds of the formula Ia can also be carried out by treatment with bases in inert solvents, for example by treatment with sodium hydride in an aprotic solvent such as dimethylformamide.

During the cyclization, guanidino groups can be blocked by protective groups, for example NO₂. Amino groups can be present in protected form or still as an NO₂ or cyano function which can later be reduced to the amino group or, in the case of the cyano group, also be converted into the formamidino group.

Compounds of the formula I in which the 5-membered ring heterocycle is a dioxo- or thioxo-oxo-substituted imidazolidine ring in which W is R¹-A-C(R¹³) and c is 1 can also be obtained by reacting a compound of the formula VII with an isocyanate or isothiocyanate of the formula X

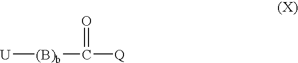

in which B, U and b are defined as indicated above for the formula VIII and Q is an alkoxy group, for example a (C₁-C₄)-alkoxy group such as methoxy, ethoxy or tert-butoxy, a (C₆-C₁₄)-aryloxy group, for example phenoxy, or a (C₆-C₁₄)-aryl-(C₁-C₄)-alkoxy group, for example benzyloxy. In this case, a compound of the formula XI

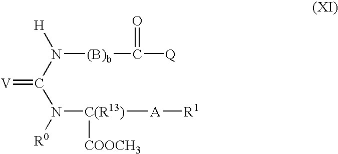

is obtained in which A, B, V, Q, R⁰, R¹, R¹³ and b are defined as indicated above for the formulae IX and X, which is then cyclized under the influence of an acid or of a base, such as described above for the cyclization of the compounds of the formula IX to a compound of the formula XII

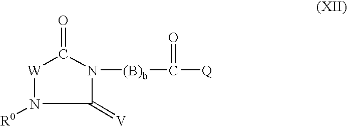

in which B, Q, V, W, R⁰ and b are defined as indicated above for the formulae Ia and X. From the compound of the formula XII, a compound of the formula Ia is then obtained by hydrolysis of the group CO-Q to give the carboxylic acid COOH and subsequent coupling with a compound of the formula III, as described above for the coupling of the compounds of the formulae II and III. Here too, during the cyclization functional groups can be present in protected form or in the form of precursors, for example guanidino groups are blocked by NO₂ or amino groups are present in protected form or still as an NO₂ or cyano function which can later be reduced to the amino group or, in the case of the cyano group, also be converted into the formamidino group.

A further method for the preparation of compounds of the formula Ia is, for example, the reaction of compounds of the formula XIII,

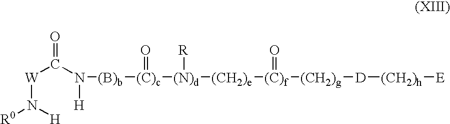

in which W is R¹-A-C(R¹³) and for which otherwise the definitions indicated above apply, with phosgene, thiophosgene or corresponding equivalents (analogously to S. Goldschmidt and M. Wick, Liebigs Ann. Chem. 575 (1952), 217-231 and C. Tropp, Chem. Ber. 61 (1928), 1431-1439).

The conversion of an amino function into an guanidino function can be carried out using the following reagents:

1. O-Methylisourea (S. Weiss and H. Krommer, Chemiker Zeitung 98 (1974), 617-618)

2. S-Methylisothiourea (R. F. Borne, M. L. Forrester and I. W. Waters, J. Med. Chem. 20 (1977), 771-776)

3. Nitro-S-methylisothiourea (L. S. Hafner and R. E. Evans, J. Org. Chem. 24 (1959) 57)

4. Formamidinosulfonic acid (K. Kim, Y.-T. Lin and H. S. Mosher, Tetra. Lett. 29 (1988), 3183-3186)

5. 3,5-Dimethyl-1-pyrazolylformamidinium nitrate (F. L. Scott, D. G. O'Donovan and J. Reilly, J. Amer. Chem. Soc. 75 (1953), 4053-4054)

6. N,N′-Di-tert-butyloxycarbonyl-S-methylisothiourea (R. J. Bergeron and J. S. McManis, J. Org. Chem. 52 (1987), 1700-1703)

7. N-Alkoxycarbonyl-, N,N′-dialkoxycarbonyl-, N-alkylcarbonyl- and N,N′-dialkylcarbonyl-S-methylisothiourea (H. Wollweber, H. Kolling, E. Niemers, A. Widdig, P. Andrews, H.-P. Schulz and H. Thomas, Arzneim. Forsch./Drug Res. 34 (1984), 531-542).

Amidines can be prepared from the corresponding cyano compounds by addition of alcohols (for example methanol or ethanol) in acidic anhydrous medium (for example dioxane, methanol or ethanol) and subsequent aminolysis, for example treatment with ammonia in alcohols such as, for example, isopropanol, methanol or ethanol (G. Wagner, P. Richter and Ch. Garbe, Pharmazie 29 (1974), 12-55). A further method of preparing amidines is the addition of H₂S to the cyano group, followed by methylation of the resulting thioamide and subsequent reaction with ammonia (GDR Patent No. 235 866).

With respect to the preparation of the compounds of the formula I, the disclosure of the following publications is fully incorporated by reference: WO-A-94/21607, WO-A-95/14008, EP-A-449 079, EP-A-530 505 (U.S. Pat. No. 5,389,614), WO-A-93/18057, EP-A-566 919 (U.S. Pat. No. 5,397,796), EP-A-580 008 (U.S. Pat. No. 5,424,293) and EP-A-584 694 (U.S. Pat. No. 5,554,594) as well as WO-A-96/33976.

The compounds of the formula I are antagonists of the adhesion receptor VLA-4. They have the ability to inhibit cell-cell and cell-matrix interaction processes in which interactions between VLA-4 and its ligands play a part. The activity of the compounds of the formula I can be demonstrated, for example, in an assay in which the binding of cells which contain the VLA-4 receptor, for example leucocytes, to ligands of this receptor is measured, for example to VCAM-1, which for this purpose can advantageously also be prepared by genetic engineering. Details of such an assay are described below. In particular, the compounds of the formula I are able to inhibit the adhesion and the migration of leucocytes, for example the adhesion of leucocytes to endothelial cells which—as explained above—is controlled via the VCAM-1/VLA-4 adhesion mechanism.

The compounds of the formula I and their physiologically tolerable salts are therefore suitable for the treatment and prophylaxis of diseases which are based on the interaction between the VLA-4 receptor and its ligands or can be influenced by inhibition of this interaction, and in particular they are suitable for the treatment and prophylaxis of diseases which are caused at least partially by an undesired extent of leucocyte adhesion and/or leucocyte migration or are associated therewith, and for whose prevention, alleviation or cure the adhesion and/or migration of leucocytes should be decreased. They can thus be employed, for example, as antiinflammatory agents in the case of inflammatory symptoms having very different causes. The compounds of the formula I according to the present invention are used, for example, for the treatment or prophylaxis of rheumatoid arthritis, inflammatory bowel disease (ulcerative colitis), systemic lupus erythematosus or for the treatment or prophylaxis of inflammatory disorders of the central nervous system, such as, for example, multiple sclerosis, for the treatment or prophylaxis of asthma or of allergies, for example allergies of the delayed type (type IV allergy), furthermore for the treatment or prophylaxis of cardiovascular disorders, arteriosclerosis, restenosis, for the treatment or prophylaxis of diabetes, for the prevention of damage to organ transplants, for the inhibition of tumor growth or tumor metastasis in various malignancies, for the therapy of malaria, and also of other diseases in which blocking of the integrin VLA-4 and/or influencing of the leucocyte activity appears appropriate for prevention, alleviation or cure. The compounds of the formula I and their salts can furthermore be employed for diagnostic purposes, e.g. in in vitro diagnoses, and as tools in biochemical investigations in which VLA-4 blocking or influencing of cell-cell or cell-matrix interactions is intended.

The compounds of the formula I and their physiologically tolerable salts can be administered according to the invention, as pharmaceuticals for therapy or prophylaxis, to animals, preferably to mammals, and in particular to man. They can be administered per se, in mixtures with one another or in the form of pharmaceutical preparations which permit enteral or parenteral use and which as active constituents contain an efficaceous dose of at least one compound of the formula I and/or its physiologically tolerable salts in addition to customary, pharmaceutically innocuous excipients and/or additives. The present invention also relates to the use of pharmaceutical preparations which contain one or more compounds of the formula I and/or their physiologically tolerable salts for the abovementioned inventive uses of the compounds of the formula I. The pharmaceutical preparations normally contain approximately 0.5 to 90% by weight of the therapeutically active compounds of the formula I and/or their physiologically tolerable salts.

The pharmaceuticals can be administered orally, for example in the form of pills, tablets, film-coated tablets, sugar-coated tablets, granules, hard and soft gelatin capsules, solutions, syrups, emulsions or suspensions. However, administration can also take place rectally, for example in the form of suppositories, or parenterally, for example in the form of injection or infusion solutions, microcapsules or rods, or percutaneously, for example in the form of ointments or tinctures, or by other routes, for example in the form of nasal sprays or aerosol mixtures.

The pharmaceutical preparations to be employed according to the invention are prepared in a manner known per se, pharmaceutically inert inorganic and/or organic excipients being used in addition to the compound(s) of the formula I and/or its/their physiologically tolerable salts. For the production of pills, tablets, sugar-coated tablets and hard gelatin capsules, it is possible to use, for example, lactose, corn starch or derivatives thereof, talc, stearic acid or its salts, etc. Excipients for soft gelatin capsules and suppositories are, for example, fats, waxes, semisolid and liquid polyots, natural or hardened oils, etc. Suitable excipients for the preparation of solutions, for example injection solutions, or of emulsions or syrups are, for example, water, alcohols, glycerol, polyols, sucrose, invert sugar, glucose, vegetable oils, etc. Suitable excipients for microcapsules, implants or rods are, for example, copolymers of glycolic acid and lactic acid.

In addition to the active compounds and excipients the pharmaceutical preparations can additionally contain additives, such as, for example, fillers, disintegrants, binders, lubricants, wetting agents, stabilizers, emulsifiers, preservatives, sweeteners, colorants, flavorings or aromatizers, thickeners, diluents, buffer substances, and also solvents or solubilizers or agents for achieving a depot effect, as well as salts for changing the osmotic pressure, coatings or antioxidants. They can also contain two or more compounds of the formula I and/or their physiologically tolerable salts. In addition to at least one compound of the formula I and/or its physiologically tolerable salts, they can further contain one or more other therapeutically or prophylactically active substances, for example substances having antiinflammatory action.

The dose can vary within wide limits and is to be adapted to the individual conditions in each individual case. In general, in the case of oral administration a daily dose of approximately 0.01 to 100 mg/kg, preferably 0.1 to 10 mg/kg, in particular 0.3 to 2 mg/kg of body weight is adequate to achieve effective results. In the case of intravenous administration, the daily dose is in general approximately 0.01 to 50 mg/kg, preferably 0.01 to 10 mg/kg of body weight. The daily dose can be subdivided, in particular in the case of administration of relatively large amounts, into a number of, for example 2, 3 or 4, part administrations. Where appropriate, it may be necessary, depending on individual behavior, to deviate upwards or downwards from the indicated daily dose. Pharmaceutical preparations normally contain 0.2 to 500 mg, preferably 1 to 100 mg, of active compound of the formula I and/or its physiologically tolerable salts per dose.

Certain compounds of the formula I have still not been disclosed in the prior art. The present invention also relates to these novel compounds per se.

The present invention thus also relates on the one hand to compounds of the formula Ib per se,

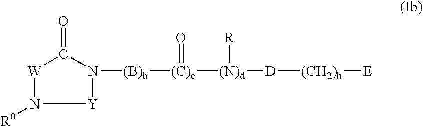

in which

W is R¹-A-CH or R¹-A-CH═C;

Y is a carbonyl, thiocarbonyl or methylene group;

A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, phenylene-(C₂-C₆)-alkenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent radical from the group consisting of (C₁-C₆)-alkylene, (C₂-C₆)-alkenylene, phenylene, phenylene-(C₁-C₃)-alkyl, (C₁-C₃)-alkylenephenyl;

D is C(R²)(R³);

E is tetrazolyl, (R⁸O)₂P(O), HOS(O)₂, R⁹NHS(O)₂ or R¹⁰CO;

R is hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R⁰ is (C₇-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R¹ is X—NH—C(═NH)—(CH₂)ₚ or X¹—NH—(CH₂)ₚ where p is one of the numbers 0, 1, 2 and 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″), where R′ and R″ independently of one another have the meanings of X;

R² is hydrogen or phenyl;

R³ is hydrogen, COOR⁴, CON(CH₃)R⁴ or CONHR⁴;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino and trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

b, c and d independently of one another can be 0 or 1, but cannot all simultaneously be 0;

h is one of the numbers 0, 1, 2, 3, 4, 5 and 6;

in all their stereoisomeric forms and mixtures thereof in any ratio, and their physiologically tolerable salts.

All the above explanations for formula I, for example with respect to alkyl radicals, aryl radicals, etc., also apply to the compounds of the formula Ib correspondingly. The above preferred meanings also apply here correspondingly.

The above explanations for the preparation of the compounds of the formula I and their use likewise also apply to the compounds of the formula Ib. These compounds, of course, are also inhibitors of leucocyte adhesion and/or antagonists of the VLA-4 receptor and are suitable for the treatment and prophylaxis of diseases which are caused by an undesired extent of leucocyte adhesion and/or leucocyte migration or which are associated therewith or in which cell-cell or cell-matrix interactions which are based on interactions of VLA-4 receptors with their ligands play a part, for example of inflammatory processes. The present invention furthermore relates to the compounds of the formula Ib for use as pharmaceuticals and to pharmaceutical preparations which contain one or more compounds of the formula Ib and/or their physiologically tolerable salts in addition to pharmaceutically innocuous excipients and/or additives, the above explanations also applying to these pharmaceutical preparations in turn.

Certain compounds of the formula I are not explicitly disclosed in the prior art and thus represent a selection of the variety of compounds covered by WO-A-95/14008. The present invention also relates to these novel compounds per se. Thus, the present invention also relates, on the other hand, to compounds of the formula Ic per se,

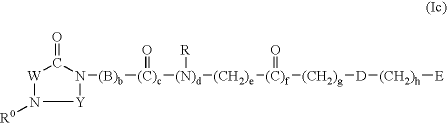

in which

W is R¹-A-C(R¹³);

Y is a carbonyl, thiocarbonyl or methylene group;

A is a phenylene radical;

B is a bivalent radical from the group consisting of (C₁-C₆)-alkylene, (C₂-C₆)-alkenylene, phenylene, phenylene-(C₁-C₃)-alkyl, (C₁-C₃)-alkylenephenyl;

D is C(R²)(R³), N(R³) or CH═C(R³);

E is tetrazolyl, (R⁸O)₂P(O), HOS(O)₂, R⁹NHS(O)₂ or R¹⁰CO;

R is hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R⁰ is (C₆-C₁₄)-aryl-(C₁-C₈)-alkyl optionally substituted in the aryl radical;

R¹ is X—NH—C(═NH)—(CH₂)ₚ or X¹—NH—(CH₂)ₚ, where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy, which can also be substituted in the aryl radical, or amino;

X¹ has one of the meanings of X or is R′—NH—C(═N—R″), where R′ and R″ independently of one another have the meanings of X;

R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;

R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R⁴, CONHR¹⁴, CSNHR⁴, COOR¹⁵, CON(CH₃)R¹⁵ or CONHR¹⁵;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino or trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹³ is (C₁-C₆)-alkyl, (C₆-C₁₄)-aryl-(C₁-C₈)-alkyl optionally substituted in the aryl radical or (C₃-C₈)-cycloalkyl;

R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;

R¹⁵ is R¹⁶—(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo;

b, c, d and f independently of one another are 0 or 1, but cannot all simultaneously be 0;

e, g and h independently of one another are 0, 1, 2, 3, 4, 5 or 6;

in all their stereoisomeric forms and mixtures thereof in any ratio, and their physiologically tolerable salts.

All the above explanations for the formula I, for example with respect to alkyl radicals, aryl radicals, etc., also apply to the compounds of the formula Ic correspondingly. The above preferred meanings also apply here correspondingly. In addition, particularly preferably, in the compounds of the formula Ic, independently of one another, b is 1, c is 1, d is 1, f is 0 and g is 0. e and h are particularly preferably independently of one another 0 or 1. It is also particularly preferred if Y in the compounds of the formula Ic is a carbonyl group.

The above explanations for the preparation of the compounds of the formula I and their use likewise also apply to the compounds of the formula Ic. These compounds, of course, are also inhibitors of leucocyte adhesion and/or antagonists of the VLA-4 receptor and are suitable for the treatment and prophylaxis of diseases which are caused by an undesired extent of leucocyte adhesion and/or leucocyte migration or which are associated therewith or in which cell-cell or cell-matrix interactions which are based on interactions of VLA-4 receptors with their ligands play a part, for example of inflammatory processes. The present invention furthermore relates to the compounds of the formula Ic for use as pharmaceuticals and to pharmaceutical preparations which contain one or more compounds of the formula Ic and/or their physiologically tolerable salts in addition to pharmaceutically innocuous excipients and/or additives, the above explanations also applying to these pharmaceutical preparations in turn.

Furthermore, in the prior art still no compounds of the formula I are explicitly disclosed in which b is 1 and B is a substituted alkylene radical. The present invention thus also relates to compounds of the formula Id per se,

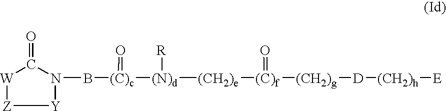

in which

W is R¹-A-C(R³) or R¹-A-CH═C;

Y is a carbonyl, thiocarbonyl or methylene group;

Z is N(R⁰);

A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, phenylene-(C₂-C₆)-alkenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent (C₁-C₆)-alkylene radical which is substituted by a radical from the group consisting of (C₁-C₈)-alkyl, (C₂-C₈)-alkenyl, (C₂-C₈)-alkynyl, (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl and heteroaryl-(C₁-C₆)-alkyl optionally substituted in the heteroaryl radical;

D is C(R²)(R³), N(R³) or CH═C(R³);

E is tetrazolyl, (R⁸O)₂P(O), HOS(O)₂, R⁹NHS(O)₂ or R¹⁰CO;

R is hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R⁰ is hydrogen, (C₁-C₈)-alkyl, (C₃-C₁₂)-cycloalkyl, (C₃-C₁₂)-cycloalkyl-(C₁-C₈)-alkyl, (C₆-C₁₂)-bicycloalkyl, (C₆-C₁₂)-bicycloalkyl-(C₁-C₈)-alkyl, (C₆-C₁₂)-tricycloalkyl, (C₆-C₁₂)-tricycloalkyl-(C₁-C₈)-alkyl, optionally substituted (C₆-C₁₄)-aryl, (C₆-C₁₄)-aryl-(C₁-C₈)-alkyl optionally substituted in the aryl radical, optionally substituted heteroaryl, heteroaryl-(C₁-C₈)-alkyl optionally substituted in the heteroaryl radical, CHO, (C₁-C₈)-alkyl-CO, (C₃-C₁₂)-cycloalkyl-CO, (C₃-C₁₂)-cycloalkyl-(C₁-C₈)-alkyl-CO, (C₆-C₁₂)-bicycloalkyl-CO, (C₆-C₁₂)-bicycloalkyl-(C₁-C₈)-alkyl-CO, (C₆-C₁₂)-tricycloalkyl-CO, (C₆-C₁₂)-tricycloalkyl-(C-1-C₈)-alkyl-CO, optionally substituted (C₆-C₁₄)-aryl-CO, (C₆-C₁₄)-aryl-(C₁-C₈)-alkyl-CO optionally substituted in the aryl radical, optionally substituted heteroaryl-CO, heteroaryl-(C₁-C₈)-alkyl-CO optionally substituted in the heteroaryl radical, (C₁-C₈)-alkyl-S(O)ₙ, (C₃-C₁₂)-cycloalkyl-S(O)ₙ, (C₃-C₁₂)-cycloalkyl-(C₁-C₈)-alkyl-S(O)ₙ, (C₆-C₁₂)-bicycloalkyl-S(O)ₙ, (C₆-C₁₂)-bicycloalkyl-(C₁-C₈)-alkyl-S(O)ₙ, (C₆-C₁₂)-tricycloalkyl-S(O)ₙ, (C₆-C₁₂)-tricycloalkyl-(C₁-C₈)-alkyl-S(O)ₙ, optionally substituted (C₆-C₁₄)-aryl-S(O)ₙ, (C₆-C₁₄)-aryl-(C₁-C₈)-alkyl-S(O)ₙ optionally substituted in the aryl radical, optionally substituted heteroaryl-S(O)ₙ or heteroaryl-(C₁-C₈)-alkyl-S(O)ₙ optionally substituted in the heteroaryl radical, where n is 1 or 2;

R¹ is X—NH—C(═NH)—(CH₂)ₚ or X¹—NH—(CH₂)ₚ, where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″), where R′ and R″ independently of one another have the meanings of X;

R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;

R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R⁴, CONHR¹⁴, CSNHR¹⁴, COOR¹⁵, CON(CH₃)R¹⁵ or CONHR¹⁵;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino and trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated) or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹³ is hydrogen, (C₁-C₆)-alkyl, (C₆-C₁₄)-aryl-(C₁-C₈)-alkyl optionally substituted in the aryl radical or (C₃-C₈)-cycloalkyl;

R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;

R¹⁵ is R¹⁶—(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo;

c, d and f independently of one another are 0 or 1, but cannot all simultaneously be 0;

e, g and h independently of one another are 0, 1, 2, 3, 4, 5 or 6;

in all their stereoisomeric forms and mixtures thereof in any ratio, and their physiologically tolerable salts.

All above explanations for the formula I, for example with respect to alkyl radicals, aryl radicals, etc., also apply to the compounds of the formula Id correspondingly. The above preferred meanings also apply here correspondingly. In addition, particularly preferably, in the compounds of the formula Id, independently of one another, c is 1, d is 1, f is 0 and g is 0. e and h are particularly preferably independently of one another 0 or 1. With respect to the group B, in addition the following applies to the compounds of the formula Id.

The (C₁-C₆)-alkylene radical representing the group B in the compounds of the formula Id is preferably a (C₁-C₄)-alkylene radical, particularly preferably a methylene radical or an ethylene radical (=1,2-ethylene), very particularly preferably a methylene radical. The substituent on the group B can on the one hand contain a cyclic system when it is a substituent from the group consisting of (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl and heteroaryl-(C₁-C₆)-alkyl optionally substituted in the heteroaryl radical, and can on the other hand be acyclic when it is a substituent from the group consisting of (C₁-C₈)-alkyl, (C₂-C₈)-alkenyl and (C₂-C₈)-alkynyl. These acyclic substituents can each contain 2, 3, 4, 5, 6, 7 or 8 carbon atoms or, in the case of the saturated alkyl radical, also 1 carbon atom. In the case of the alkenyl radicals and alkynyl radicals, the double bond or triple bond can be located in any desired position and in the case of the double bond can have the cis configuration or trans configuration. As explained above, these alkyl radicals, alkenyl radicals and alkynyl radicals can be straight-chain or branched. As examples of substituents which the (C₁-C₆)-alkylene radical representing B can carry the following are mentioned: methyl, ethyl, n-propyl, n-butyl, n-pentyl, n-hexyl, n-heptyl, n-octyl, isopropyl, isobutyl, isopentyl, isohexyl, sec-butyl, tert-butyl, tert-pentyl, neopentyl, neohexyl, 3-methylpentyl, 2-ethylbutyl, vinyl, allyl, 1-propenyl, 2-butenyl, 3-butenyl, 3-methyl-2-butenyl, ethynyl, 1-propynyl, 2-propynyl, 6-hexynyl, phenyl, benzyl, 1-phenylethyl, 2-phenylethyl, 3-phenylpropyl, 4-biphenylylmethyl, cyclopropyl, cyclopropylmethyl, cyclopentyl, cyclohexyl, cyclohexylmethyl, 2-cyclohexylethyl, 3-cyclooctylpropyl, 2-pyridyl, 3-pyridyl, 4-pyridyl, 4-pyridylmethyl, 2-(4-pyridyl)ethyl, 2-furylmethyl, 2-thienylmethyl, 3-thienylmethyl or 2-(3-indolyl)ethyl.

Preferred compounds of the formula Id are those in which simultaneously

W is R¹-A-C(R¹³);

Y is a carbonyl group;

Z is N(R⁰);

A is a bivalent radical from the group consisting of (C₃-C₇)-cycloalkylene, phenylene, phenylene-(C₁-C₆)-alkyl, (C₁-C₆)-alkylene phenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent methylene radical or ethylene radical which is substituted by a radical from the group consisting of (C₁-C₈)-alkyl, (C₂-C₈)-alkenyl, (C₂-C₈)-alkynyl, (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl and heteroaryl-(C₁-C₆)-alkyl optionally substituted in the heteroaryl radical;

D is C(R²)(R³);

E is tetrazolyl or R¹⁰CO;

R is hydrogen or (C₁-C₈)-alkyl;

R⁰ is hydrogen, (C₁-C₈)-alkyl, (C₃-C₁₂)-cycloalkyl, (C₃-C₁₂)-cycloalkyl-(C₁-C₈)-alkyl, (C₆-C₁₂)-bicycloalkyl, (C₆-C₁₂)-bicycloalkyl-(C₁-C₈)-alkyl, (C₆-C₁₂)-tricycloalkyl, (C₆-C₁₂)-tricycloalkyl-(C₁-C₈)-alkyl, optionally substituted (C₆-C₁₄)-aryl, (C₆-C₁₄)-aryl-(C₁-C₈)-alkyl optionally substituted in the aryl radical, optionally substituted heteroaryl, heteroaryl —(C₁-C₈)-alkyl optionally substituted in the heteroaryl radical, CHO, (C₁-C₈)-alkyl-CO, (C₃-C₁₂)-cycloalkyl-CO, (C₃-C₁₂)-cycloalkyl-(C₁-C₈)-alkyl-CO, (C₆-C₁₂)-bicycloalkyl-CO, (C₆-C₁₂)-bicycloalkyl-(C₁-C₈)-alkyl-CO, (C₆-C₁₂)-tricycloalkyl-CO, (C₆-C₁₂)-tricycloalkyl-(C₁-C₈)-alkyl-CO, optionally substituted (C₆-C₁₄)-aryl-CO, (C₆-C₁₄)-aryl-(C₁-C₈)-alkyl-CO optionally substituted in the aryl radical, optionally substituted heteroaryl-CO, heteroaryl-(C₁-C₈)-alkyl-CO optionally substituted in the heteroaryl radical, (C₁-C₈)-alkyl-S(O)ₙ, (C₃-C₁₂)-cycloalkyl-S(O)ₙ, (C₃-C₁₂)-cycloalkyl-(C₁-C₈)-alkyl-S(O)ₙ, (C₆-C₁₂)-bicycloalkyl-S(O)ₙ, (C₆-C₁₂)-bicycloalkyl-(C₁-C₈)-alkyl-S(O)ₙ, (C₆-C₁₂)-tricycloalkyl-S(O)ₙ, (C₆-C₁₂)-tricycloalkyl-(C₁-C₈)-alkyl-S(O)ₙ, optionally substituted (C₆-C₁₄)-aryl-S(O)ₙ, (C₆-C₁₄)-aryl-(C₁-C₈)-alkyl-S(O)ₙ optionally substituted in the aryl radical, optionally substituted heteroaryl-S(O)ₙ or heteroaryl-(C₁-C₈)-alkyl-S(O), optionally substituted in the heteroaryl radical, where n is 1 or 2;

R¹ is X—NH—C(═NH)—(CH₂)ₚ or X¹—NH—(CH₂)ₚ where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″), where R′ and R″ independently of one another have the meanings of X;

R² is hydrogen or (C₁-C₈)-alkyl;

R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, CON(CH₃)R⁴, CONHR¹⁴, CON(CH₃)R¹⁵ or CONHR¹⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, or a radical R⁶CO—, where the aryl radical and, independently thereof, the heterocyclic radical, can be mono- or polysubstituted by identical or different radicals from the group consisting of (C₁-C₈)-alkyl, (C₁-C₈)-alkoxy, halogen, nitro, amino or trifluoromethyl;

R⁶ is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical, and their esters and amides, where free functional groups can be protected by protective groups customary in peptide chemistry;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is R¹²CO, optionally substituted (C₆-C₁₄)-aryl-S(O)₂ or (C₁-C₁₈)-alkyl-S(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical or optionally substituted (C₆-C₁₄)-aryloxy;

R¹³ is hydrogen or (C₁-C₄)-alkyl;

R¹⁴ is (C₁-C₁₀)-alkyl which can optionally be mono- or polysubstituted by identical or different radicals from the group consisting of hydroxyl, hydroxycarbonyl, aminocarbonyl, mono- or di-((C₁-C₁₈)-alkyl)amino-carbonyl, (C₆-C₁₄)-aryl-(C₁-C₈)-alkoxycarbonyl which can also be substituted in the aryl radical, (C₁-C₈)-alkoxy, (C₁-C₈)-alkoxycarbonyl, optionally substituted (C₃-C₈)-cycloalkyl, tetrazolyl-(C₁-C₃)-alkyl, trifluoromethyl and R⁵;

R¹⁵ is R¹⁶—(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo;

c and d are 1 and f is 0;

e and h independently of one another are 0 or 1 and g is 0;

in all their stereoisomeric forms and mixtures thereof in any ratio, and their physiologically tolerable salts.

Particularly preferred compounds of the formula Id are those in which the radical by which the group B is substituted is a (C₁-C₈)-alkyl radical, in all their stereoisomeric forms and mixtures thereof in any ratio, and their physiologically tolerable salts.

The above explanations for the preparation of the compounds of the formula I and their use likewise also apply to the compounds of the formula Id. These compounds, of course, are also inhibitors of leucocyte adhesion and/or antagonists of the VLA-4 receptor and are suitable for the treatment and prophylaxis of diseases which are caused by an undesired extent of leucocyte adhesion and/or leucocyte migration or which are associated therewith or in which cell-cell or cell-matrix interactions which are based on interactions of VLA-4 receptors with their ligands play a part, for example of inflammatory processes. The present invention furthermore relates to the compounds of the formula Id for use as pharmaceuticals and to pharmaceutical preparations which contain one or more compounds of the formula Id and/or their physiologically tolerable salts in addition to pharmaceutically innocuous excipients and/or additives, the above explanations also applying to these pharmaceutical preparations in turn.

In the prior art, no compounds of the formula I are disclosed in which R⁰ is an acyl radical, sulfonyl radical or sulfinyl radical. The present invention thus furthermore also relates to compounds of the formula Ie per se,

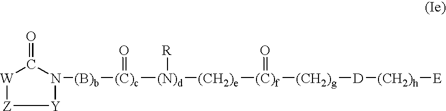

in which

W is R¹-A-C(R¹³) or R¹-A-CH═C;

Y is a carbonyl, thiocarbonyl or methylene group;

Z is N(R⁰), oxygen, sulfur or a methylene group;

A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, phenylene-(C₂-C₆)-alkenyl or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;

B is a bivalent radical from the group consisting of (C₁-C₆)-alkylene, (C₂-C₆)-alkenylene, phenylene, phenylene-(C₁-C₃)-alkyl, (C₁-C₃)-alkylenephenyl;

D is C(R²)(R³), N(R³) or CH═C(R³);

E is tetrazolyl, (R⁸O)₂P(O), HOS(O)₂, R⁹NHS(O)₂ or R¹⁰CO;

R is hydrogen, (C₁-C₈)-alkyl, (C₃-C₈)-cycloalkyl, optionally substituted (C₆-C₁₄)-aryl or (C₆-C₁₄)-aryl-(C₁-C₈)-alkyl optionally substituted in the aryl radical;

R⁰ is CHO, (C₁-C₈)-alkyl-CO, (C₃-C₁₂)-cycloalkyl-CO, (C₃-C₁₂)-cycloalkyl-(C₁-C₈)-alkyl-CO, (C₆-C₁₂)-bicycloalkyl-CO, (C₆-C₁₂)-bicycloalkyl-(C₁-C₈)-alkyl-CO, (C₆-C₁₂)-tricycloalkyl-CO, (C₆-C₁₂)-tricycloalkyl-(C₁-C₈)-alkyl-CO, optionally substituted (C₆-C₁₄)-aryl-CO, (C₆-C₁₄)-aryl-(C₁-C₈)-alkyl-CO optionally substituted in the aryl radical, optionally substituted heteroaryl-CO, heteroaryl-(C₁-C₈)-alkyl-CO optionally substituted in the heteroaryl radical, (C₁-C₈)-alkyl-S(O)ₙ, (C₃-C₁₂)-cycloalkyl-S(O)ₙ, (C₃-C₁₂)-cycloalkyl-(C₁-C₈)-alkyl-S(O)ₙ, (C₆-C₁₂)-bicycloalkyl-S(O)ₙ, (C₆-C₁₂)-bicycloalkyl-(C₁-C₈)-alkyl-S(O)ₙ, (C₆-C₁₂)-tricycloalkyl-S(O)ₙ, (C₆-C₁₂)-tricycloalkyl-(C₁-C₈)-alkyl-S(O)ₙ, optionally substituted (C₆-C₁₄)-aryl-S(O)ₙ, (C₆-C₁₄)-aryl-(C₁-C₈)-alkyl-S(O)ₙ optionally substituted in the aryl radical, optionally substituted heteroaryl-S(O)ₙ or heteroaryl-(C₁-C₈)-alkyl-S(O)ₙ optionally substituted in the heteroaryl radical, where n is 1 or 2;

R¹ is X—NH—C(═NH)—(CH₂)ₚ or X¹—NH—(CH₂)ₚ, where p is 0, 1, 2 or 3;

X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;

X¹ has one of the meanings of X or is R′—NH—C(═N—R″), where R′ and R″ independently of one another have the meanings of X;

R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;

R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R¹⁴, CONHR¹⁴, CSNHR¹⁴, COOR¹⁵, CON(CH₃)R¹⁵ or CONHR¹⁵;

R⁴ is hydrogen or (C₁-C₂₈)-alkyl which can optionally be mono- or polysubstituted by identical or different radicals R^{4′};

R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl or the radical R⁵;

R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated or completely hydrogenated and which can contain one, two or three identical or different heteroatoms from the group consisting of nitrogen, oxygen and sulfur, a radical R⁶ or a radical R⁶CO—, where the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino or trifluoromethyl;

R⁶ is R⁷R⁸N, R⁷⁰ or R⁷S or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—, and their esters and amides, where hydrogen or hydroxymethyl can optionally stand in place of free functional groups and/or where free functional groups can be protected by protective groups customary in peptide chemistry;

R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, where the alkyl groups can optionally be substituted by an amino group and/or where the aryl radicals can be mono- or polysubstituted, preferably monosubstituted, by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or in which the peptide bond can be reduced to —NH—CH₂—;

R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;

R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;

R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;

R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;

R¹³ is hydrogen, (C₁-C₆)-alkyl, (C₆-C₁₄)-aryl-(C₁-C₈)-alkyl optionally substituted in the aryl radical or (C₃-C₈)-cycloalkyl;

R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;

R¹⁵ is R¹⁶—(C₁-C₆)-alkyl or R¹⁶;

R¹⁶ is a 6- to 24-membered bicyclic or tricyclic radical which is saturated or partially unsaturated and which can also contain one to four identical or different heteroatoms from the group consisting of nitrogen, oxygen and sulfur and which can also be substituted by one or more identical or different substituents from the group consisting of (C₁-C₄)-alkyl and oxo;

b, c, d and f independently of one another are 0 or 1, but cannot all simultaneously be 0;

e, g and h independently of one another are 0, 1, 2, 3, 4, 5 or 6;

in all their stereoisomeric forms and mixtures thereof in any ratio, and their physiologically tolerable salts.

All above explanations for the formula I, for example with respect to alkyl radicals, aryl radicals, etc., also apply to the compounds of the formula Ie accordingly. The above preferred meanings also apply here correspondingly.

The above explanations for the preparation of the compounds of the formula I and their use likewise also apply to the compounds of the formula Ie. These compounds, of course, are also inhibitors of leucocyte adhesion and/or antagonists of the VLA-4 receptor and are suitable for the treatment and prophylaxis of diseases which are caused by an undesired extent of leucocyte adhesion and/or leucocyte migration or are associated therewith or in which cell-cell or cell-matrix interactions which are based on interactions of VLA-4 receptors with their ligands play a part, for example of inflammatory processes. The present invention furthermore relates to the compounds of the formula Ie for use as pharmaceuticals and to pharmaceutical preparations which contain one or more compounds of the formula Ie and/or their physiologically tolerable salts in addition to pharmaceutically innocuous excipients and/or additives, the above explanations also applying to these pharmaceutical preparations in turn.

### EXAMPLES

The products were identified by means of mass spectra (MS) and/or NMR spectra.

Compounds which were purified by chromatography using an eluent which contained, for example, acetic acid or trifluoroacetic acid and were then freeze-dried partly still contained, depending on the freeze-drying procedure, the acid contained in the eluent, and were thus partially or completely obtained in the form of a salt of the acid used, for example in the form of the acetic acid salt or trifluoroacetic acid salt.

The abbreviations have the following meanings:

| | |
|---|---|
| DMF | N,N-Dimethylformamide |
| THF | Tetrahydrofuran |
| DCC | N,N′-Dicyclohexylcarbodiimide |
| HOBt | 1-Hydroxybenzotriazole |
| HOOBt | 3-Hydroxy-4-oxo-3,4-dihydro-1,2,3-benzotriazine |

### Example 1

((R,S)-4-(4-(Amino-imino-methyl)phenyl)-4-methyl-2,5-dioxoimidazolidin-1-yl)acetyl-L-aspartyl-L-phenylglycine

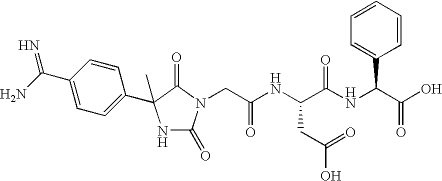

1a) (R,S)-4-(4-Cyanophenyl)-4-methyl-2,5-dioxoimidazolidine

20 g (138 mmol) of p-acetylbenzonitrile, 115.6 g of ammonium carbonate (1.21 mol) and 11.6 g of potassium cyanide (178 mmol) were dissolved in 600 ml of a mixture of 50% ethanol and 50% water. The mixture was stirred at 55° C. for 5 hours and allowed to stand at room temperature overnight. The solution was adjusted to a pH of 6.3 using 6 N HCl and then stirred at room temperature for 2 hours. The precipitate was filtered off with suction, washed with water and dried over phosphorus pentoxide in a high vacuum. Yield: 22.33 g (75%).

FAB-MS: 216.1 (M+H)⁺

1b) Methyl((R,S)-4-(4-cyanophenyl)-4-methyl-2,5-dioxoimidazolidin-1-yl)acetate

1.068 g of sodium (46.47 mmol) were dissolved in 110 ml of abs. methanol under nitrogen. The clear solution was treated with 10 g of (R,S)-4-(4-cyanophenyl)-4-methyl-2,5-dioxoimidazolidine (46.47 mmol) and the mixture was boiled under reflux for 2 h. 7.75 g (46.68 mmol) of potassium iodide were added and a solution of 4.53 ml of methyl chloroacetate (51.3 mmol) in 5 ml of methanol was added dropwise in the course of one hour. The mixture was heated to boiling for 6 hours, allowed to stand at room temperature overnight and concentrated. The oily residue was chromatographed on silica gel using methylene chloride/ethyl acetate (9:1). Yield: 8.81 g (66%).

FAB-MS: 288 (M+H)⁺

1c) Methyl((R,S)-4-(4-(ethoxy-imino-methyl)phenyl)-4-methyl-2,5-dioxoimidazolidin 1-yl)acetate hydrochloride

A suspension of 4 g of methyl((R,S)-4-(4-cyanophenyl)-4-methyl-2,5-dioxoimidazolidin-1-yl)acetate (13.92 mmol) in 60 ml of abs. ethanol was cooled to 0° C. Dry HCl gas was passed into the suspension, the temperature always being kept below 10° C., until the nitrile band was no longer present in the IR spectrum. The ethanolic solution was treated with 200 ml of diethyl ether and allowed to stand at 4° C. overnight. The precipitate was filtered off with suction and dried in a high vacuum.

Yield: 3.96 g (77%).

FAB-MS: 334 (M+H)⁺

1d) Methyl((R,S)-4-(4-(amino-imino-methyl)phenyl)-4-methyl-2,5-dioxoimidazolidin-1-yl)acetate hydrochloride

3.96 g of methyl((R,S)-4-(4-(ethoxy-imino-methyl)phenyl)-4-methyl-2,5-dioxoimidazolidin-1-yl)acetate hydrochloride (10.7 mmol) were suspended in 40 ml of isopropanol and treated with 11.9 ml of a 2 N solution of ammonia in isopropanol. The reaction mixture was stirred at 50° C. for 2 hours. The batch was cooled and then treated with 200 ml of diethyl ether. The precipitate was filtered off with suction and dried in a high vacuum. Yield: 3.27 g (89%).

FAB-MS: 305 (M+H)⁺

1e) ((R,S)-4-(4-(Amino-imino-methyl)phenyl)-4-methyl-2,5-dioxoimidazolidin-1-yl)acetic acid hydrochloride

3.27 g of methyl((R,S)-4-(4-(amino-imino-methyl)phenyl)-4-methyl-2,5-dioxoimidazolidin-1-yl)acetate hydrochloride (9.6 mmol) were dissolved in 50 ml of concentrated hydrochloric acid. The solution was heated to boiling for 6 hours and then concentrated. Yield: 2.73 g (87%).

FAB-MS: 291.2 (M+H)⁺

1f) ((R,S)-4-(4-(Amino-imino-methyl)phenyl)-4-methyl-2,5-dioxoimidazolidin-1-yl)acetyl-L-aspartyl-L-phenylglycine-di-tert-butyl ester hydrochloride

673 mg of DCC (3.06 mmol) were added at 0° C. to a solution of 1 g of ((R,S)-4-(4-(amino-imino-methyl)phenyl)-4-methyl-2,5-dioxoimidazolidin-1-yl)acetic acid hydrochloride (3.06 mmol), 1.27 g of H-Asp(OBu^{t})-Phg-OBu^{t} hydrochloride (3.06 mmol) and 413 mg of HOBt in 10 ml of dimethylformamide. The mixture was stirred at 0° C. for one hour and at room temperature for 4 hours. The batch was then allowed to stand in a cold store over the weekend, the precipitate was filtered off with suction and the filtrate was concentrated. For purification, the substance was chromatographed on silica gel using methylene chloride/methanol/glacial acetic acid/water (8.5:1.5:0.15:0.15). Yield: 920 mg of oil (still contained acetic acid).

FAB-MS: 651.3 (M+H)⁺

1g) ((R,S)-4-(4-(Amino-imino-methyl)phenyl)-4-methyl-2,5-dioxoimidazolidin-1-yl)acetyl-L-aspartyl-L-phenylglycine

920 mg of ((R,S)-4-(4-(Amino-imino-methyl)phenyl)-4-methyl-2,5-dioxo-imidazolidin-1-yl)acetyl-L-aspartyl-L-phenylglycine di-tert-butyl ester hydrochloride were dissolved in a mixture of 5.4 ml of trifluoroacetic acid, 0.6 ml of water and 0.6 ml of dimercaptoethane. The mixture was allowed to stand at room temperature for one hour and was concentrated in a water-jet vacuum. For purification, the substance was chromatographed on Sephadex LH20 using a mixture of glacial acetic acid, n-butanol and water. The fractions containing the pure substance were concentrated. The residue was dissolved in water and freeze-dried. Yield: 390 mg.

[α]_{D}=+1.3° (c=1, in methanol, 25° C.).

FAB-MS: 539.2 (M+H)⁺

### Example 2

((R,S)-4-(4-(Amino-imino-methyl)phenyl)-3,4-dimethyl-2,5-dioxo-imidazolidin-1-yl)acetyl-L-aspartyl-L-phenylglycine

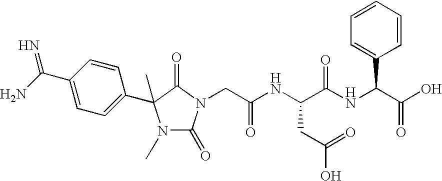

2a) Methyl((R,S)-4-(4-cyanophenyl)-3,4-dimethyl-2,5-dioxoimidazolidin-1-yl)acetate

3 g of methyl((R,S)-4-(4-cyanophenyl)-4-methyl-2,5-dioxoimidazolidin-1-yl)acetate (10.4 mmol) were dissolved in 15 ml of anhydrous dimethyl-formamide under argon. 275.5 mg of a sodium hydride dispersion in mineral oil (11.4 mmol) were added in an argon countercurrent. The reaction mixture was stirred at room temperature for 15 minutes. It was then treated with 721 μl of methyl iodide (11.4 mmol). The mixture was stirred at room temperature for 4 hours and then allowed to stand at room temperature overnight. The solution was concentrated. For purification, the substance was chromatographed on silica gel using methylene chloride/ethyl acetate (9.5:0.5). The fractions containing the pure substance were concentrated. Yield: 2.14 g of oil (68%).

FAB-MS: 302.2 (M+H)⁺

2b) Methyl((R,S)-4-(4-(ethoxy-imino-methyl)phenyl)-3,4-dimethyl-2,5-dioxoimidazolidin-1-yl)acetate hydrochloride

A solution of 2.56 g of methyl((R,S)-4-(4-cyanophenyl)-3,4-dimethyl-2,5-dioxoimidazolidin-1-yl)acetate (8.5 mmol) in 40 ml of abs. ethanol were cooled to 0° C. Dry HCl gas was passed into the solution, the temperature always being kept below 10° C., until the nitrile band was no longer present in the IR spectrum. The ethanolic solution was concentrated to 20 ml and treated with 200 ml of diethyl ether. The suspension was concentrated and dried in a high vacuum. Yield: 2.27 g (76%).

FAB-MS: 348.1 (M+H)⁺

2c) Methyl((R,S)-4-(4-(amino-imino-methyl)phenyl)-3,4-dimethyl-2,5-dioxoimidazolidin-1-yl)acetate hydrochloride

2.26 g of methyl((R,S)-4-(4-(ethoxy-imino-methyl)phenyl)-3,4-dimethyl-2,5-dioxoimidazolidin-1-yl)acetate hydrochloride (6.4 mmol) were suspended in 25 ml of isopropanol and treated with 7.2 ml of a 2 N solution of ammonia in isopropanol. The reaction mixture was stirred at 50° C. for 2.5 hours. The batch was cooled and then treated with 200 ml of diethyl ether. The precipitate was filtered off with suction and dried in a high vacuum. Yield: 1.03 g (45%).

FAB-MS: 319.4 (M+H)⁺

2d) ((R,S)-4-(4-(Amino-imino-methyl)phenyl)-3,4-dimethyl-2,5-dioxo-imidazolidin-1-yl)acetic acid hydrochloride

1 g of methyl((R,S)-4-(4-(amino-imino-methyl)phenyl)-3,4-dimethyl-2,5-dioxoimidazolidin-1-yl)acetate hydrochloride (2.8 mmol) were dissolved in 20 ml of concentrated hydrochloric acid. The solution was heated to boiling for 6 hours and then concentrated. Yield: 770 mg (81%).

FAB-MS: 305 (M+H)⁺

2e) ((R,S)-4-(4-(Amino-imino-methyl)phenyl)-3,4-dimethyl-2,5-dioxo-imidazolidin-1-yl)acetyl-L-aspartyl-L-phenylglycine di-tert-butyl ester hydrochloride

220 mg of DCC (1 mmol) were added at 0° C. to a solution of 340 mg of ((R,S)-4-(4-(amino-imino-methyl)phenyl)-3,4-dimethyl-2,5-dioxo-imidazolidin-1-yl)acetic acid hydrochloride (1 mmol), 415 mg of H-Asp(OBu^{t})-Phg-OBu^{t} hydrochloride (1 mmol) and 13.5 mg of HOBt in 7 ml of dimethylformamide. 0.13 ml of N-ethylmorpholine was added until a pH of 5.0 was achieved, and the mixture was stirred at 0° C. for one hour and at room temperature for 2 hours. The batch was then allowed to stand in a cold store over the weekend, the precipitate was filtered off with suction and the filtrate was concentrated. For purification, the substance was chromatographed on Sephadex LH20 using a mixture of glacial acetic acid, n-butanol and water. The fractions containing the pure substance were concentrated. The residue was dissolved in water and freeze-dried. Yield: 377 mg (57%).

FAB-MS: 665.2 (M+H)⁺

2f) ((R,S)-4-(4-(Amino-imino-methyl)phenyl)-3,4-dimethyl-2,5-dioxo-imidazolidin-1-yl)acetyl-L-aspartyl-L-phenylglycine

370 mg of ((R,S)-4-(4-(amino-imino-methyl)phenyl)-3,4-dimethyl-2,5-dioxoimidazolidin-1-yl)acetyl-L-aspartyl-L-phenylglycine di-tert-butyl ester hydrochloride (0.53 mmol) were dissolved in a mixture of 3.6 ml of trifluoroacetic acid, 0.4 ml of water and 0.4 ml of dimercaptoethane. The mixture was allowed to stand at room temperature for one hour and was concentrated in a water-jet vacuum. For purification, the substance was chromatographed on Sephadex LH20 using a mixture of glacial acetic acid, n-butanol and water. The fractions containing the pure substance were concentrated. The residue was dissolved in water and freeze-dried. Yield: 210 mg of a white solid (72%).

[α]_{D}=−2.8° (c=1, in methanol, 23° C.).

FAB-MS: 553.2 (M+H)⁺

The compounds of Examples 3 to 17 were prepared analogously to Example 2.

### Example 3

((R,S)-4-(4-(Amino-imino-methyl)phenyl)-3-ethyl-4-methyl-2,5-dioxoimidazolidin-1-yl)acetyl-L-aspartyl-L-phenylglycine

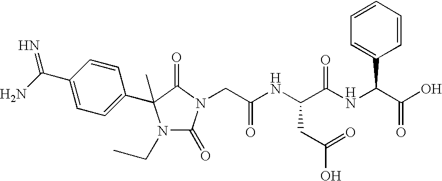

FAB-MS: 567.2 (M+H)⁺

### Example 4

((R,S)-4-(4-(Amino-imino-methyl)phenyl)-3-benzyl-4-methyl-2,5-dioxoimidazolidin-1-yl)acetyl-L-aspartyl-L-phenylglycine

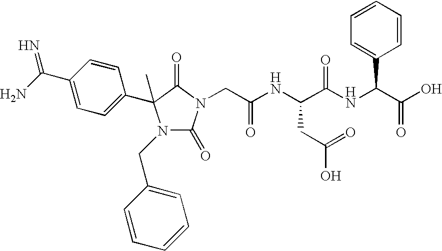

FAB-MS: 629.0 (M+H)⁺

### Example 5

((R,S)-4-(4-(Amino-imino-methyl)phenyl)-3-(4-tert-butyl-benzyl)-4-methyl-2,5-dioxoimidazolidin-1-yl)acetyl-L-aspartyl-L-phenylglycine

FAB-MS: 685.4 (M+H)⁺

### Example 6

((R,S)-4-(4-(Amino-imino-methyl)phenyl)-3-(2,3,4,5,6-pentafluorobenzyl)-4-methyl-2,5-dioxoimidazolidin-1-yl)acetyl-L-aspartyl-L-phenylglycine

FAB-MS: 719.3 (M+H)⁺

### Example 7

((R,S)-4-(4-(Amino-imino-methyl)phenyl)-3-(4-nitrobenzyl)-4-methyl-2,5-dioxoimidazolidin-1-yl)acetyl-L-aspartyl-L-phenylglycine

FAB-MS: 674.3 (M+H)⁺

### Example 8

((R,S)-4-(4-(Amino-imino-methyl)phenyl)-3-(3,5-dimethylbenzyl)-4-methyl-2,5-dioxoimidazolidin-1-yl)acetyl-L-aspartyl-L-phenylglycine

FAB-MS: 657.3 (M+H)⁺

### Example 9

((R,S)-4-(4-(Amino-imino-methyl)phenyl)-3-((2-naphthyl)methyl)-4-methyl-2,5-dioxoimidazolidin-1-yl)acetyl-L-aspartyl-L-phenylglycine

FAB-MS: 679.2 (M+H)⁺

### Example 10

((R,S)-4-(4-(Amino-imino-methyl)phenyl)-3-((2-(phenylsulfonylmethyl)-benzyl)-4-methyl-2,5-dioxoimidazolidin-1-yl)acetyl-L-aspartyl-L-phenylglycine

FAB-MS: 783.2 (M+H)⁺

### Example 11

((R,S)-4-(4-(Amino-imino-methyl)phenyl)-3-((2-biphenylyl)methyl)-4-methyl-2,5-dioxoimidazolidin-1-yl)acetyl-L-aspartyl-L-phenylglycine

FAB-MS: 705.2 (M+H)⁺

### Example 12

((R,S)-4-(4-(Amino-imino-methyl)phenyl)-3-(4-methylbenzyl)-4-methyl-2,5-dioxoimidazolidin-1-yl)acetyl-L-aspartyl-L-phenylglycine

FAB-MS: 643.3 (M+H)⁺

### Example 13

((R,S)-4-(4-(Amino-imino-methyl)phenyl)-3-((1-naphthyl)methyl)-4-methyl-2,5-dioxoimidazolidin-1-yl)acetyl-L-aspartyl-L-phenylglycine

FAB-MS: 679.3 (M+H)⁺

### Example 14

((R,S)-4-(4-(Amino-imino-methyl)phenyl)-3-((4-biphenylyl)methyl)-4-methyl-2,5-dioxoimidazolidin-1-yl)acetyl-L-aspartyl-L-phenylglycine

FAB-MS: 705.3 (M+H)⁺

### Example 15

((R,S)-4-(4-(Amino-imino-methyl)phenyl)-3-(4-trifluoromethylbenzyl)-4-methyl-2,5-dioxoimidazolidin-1-yl)acetyl-L-aspartyl-L-phenylglycine

FAB-MS: 697.3 (M+H)⁺

### Example 16

((R,S)-4-(4-(Amino-imino-methyl)phenyl)-3-(3,5-bis(trifluoromethyl)benzyl)₄-methyl-2,5-dioxoimidazolidin-1-yl)acetyl-L-aspartyl-L-phenylglycine

FAB-MS: 765.2 (M+H)⁺

### Example 17

((R,S)-4-(4-(Amino-imino-methyl)phenyl)-3-(pentamethylbenzyl)-4-methyl-2,5-dioxoimidazolidin-1-yl)acetyl-L-aspartyl-L-phenylglycine

FAB-MS: 699.3 (M+H)⁺

### Example 18

((R,S)-4-(4-(Amino-imino-methyl)phenyl)-3-((4-biphenylyl)methyl)-4-methyl-2,5-dioxoimidazolidin-1-yl)acetyl-L-N-methyl-aspartyl-L-phenylglycine

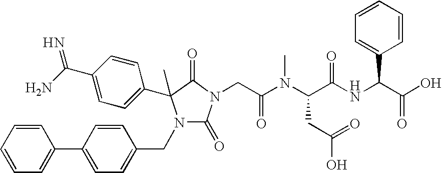

18a) Benzyl((S)-3-Benzyloxycarbonyl-5-oxo-1,3-oxazolidin-4-yl)acetate

3.57 g of β-benzyl L-N-benzyloxycarbonylaspartate (10 mmol) were dissolved in 300 ml of anhydrous toluene. 4.5 g of trioxane (50 mmol), 5.7 mg of p-toluenesulfonic acid (0.03 mmol), and 3 Å molecular sieve were added. The mixture was heated to boiling under reflux for 30 minutes and then concentrated in vacuo. For purification, the substance was chromatographed on silica gel using toluene/ethyl acetate (3:2). Yield: 2.94 g (80%).

FAB-MS: 370.2 (M+H)⁺

18b) β-Benzyl L-N-benzyloxycarbonyl-N-methylaspartate

886 mg of benzyl((S)-3-benzyloxycarbonyl-5-oxo-1,3-oxazolidin-4-yl)acetate (2.4 mmol) were dissolved in 25 ml of 1:1 mixture of methylene chloride and triethylsilane. 3 Å molecular sieve was added and 2 ml of boron trifluoride etherate were then added in portions. The reaction mixture was stirred at room temperature for 2 hours. The reaction solution was diluted with methylene chloride and the organic phase was then extracted by shaking with aqueous sodium hydrogencarbonate solution. The organic phase was concentrated and dried in vacuo. Yield: 820 mg (92%).

FAB-MS: 394.3 (M+Na)⁺

18c) L-N-Benzyloxycarbonyl-N-methylaspartyl(β-benzyl ester)-L-phenylglycine tert-butyl ester

197 mg of β-benzyl L-N-benzyloxycarbonyl-N-methylaspartate (0.5 mmol), 122 mg of H-Phg-OBu^{t} hydrochloride (0.5 mmol), 164 mg (0.5 mmol) of TOTU (0-((cyano(ethoxycarbonyl)methylene)amino)-N,N,N′,N′-tetra-methyluronium tetrafluoroborate) and 225 μl of diisopropylethylamine (1.5 mmol) were dissolved in 3 ml of anhydrous dimethylformamide at 0° C. The mixture was stirred at 0° C. for 10 minutes and at room temperature for 1.5 hours and the reaction solution was then concentrated. The residue was partitioned between ethyl acetate and 1000 ml of a KHSO₄/K₂SO₄ solution (50 g of KHSO₄ and 100 g of K₂SO₄ in 1000 ml of water). The organic phase was washed three times with a sodium hydrogencarbonate solution, with water and with saturated sodium chloride solution, dried over sodium sulfate and concentrated in vacuo. For purification, the substance was chromatographed on silica gel using toluene/ethyl acetate (10:1). Yield: 214 mg (76%).

FAB-MS: 561.3 (M+H)⁺

18d) L-N-Methyl-aspartyl-L-phenylglycine tert-butyl ester hydrochloride

2.98 g of L-N-benzyloxycarbonyl-N-methyl-aspartyl(O-benzyl ester)-L-phenylglycine tert-butyl ester (5.32 mmol) were dissolved in 300 ml of methanol and catalytically hydrogenated over Pd/active carbon at a pH of 6.5 in an autoburette with addition of 2 N methanolic HCl. The catalyst was filtered off with suction through kieselguhr and the filtrate was concentrated. The residue was triturated with diethyl ether, filtered off with suction and dried. Yield: 1.623 g (82%).

FAB-MS: 337.3 (M+H)⁺

18e) ((R,S)-4-(4-(Amino-imino-methyl)phenyl)-3-((4-biphenylyl)methyl)-4-methyl-2,5-dioxoimidazolidin-1-yl)acetyl-L-N-methyl-aspartyl-L-phenylglycine tert-butyl ester

55 mg of DCC (0.25 mmol) were added at 0° C. to a solution of 123 mg of ((R,S)-4-(4-(amino-imino-methyl)phenyl)-3-((4-biphenyl)methyl)-4-methyl-2,5-dioxoimidazolidin-1-yl)acetic acid hydrochloride (0.25 mmol), 84 mg of H-(N-methyl-Asp)-Phg-OBu^{t} hydrochloride (0.25 mmol) and 33.8 mg of HOBt (0.25 mmol) in 5 ml of dimethylformamide. The mixture was stirred at 0° C. for one hour and at room temperature for 4 hours. The batch was then allowed to stand at room temperature overnight, the precipitate was filtered off with suction and the filtrate was concentrated. Yield: 270 mg of crude product.

FAB-MS: 775.2 (M+H)⁺

18f) ((R,S)-4-(4-(Amino-imino-methyl)phenyl)-3-((4-biphenylyl)methyl)-4-methyl-2,5-dioxoimidazolidin-1-yl)acetyl-L-N-methyl-aspartyl-L-phenylglycine

270 mg of ((R,S)-4-(4-(amino-imino-methyl)phenyl)-3-((4-biphenylyl)methyl)-4-methyl-2,5-dioxoimidazolidin-1-yl)acetyl-L-N-methyl-aspartyl-L-phenylglycine tert-butyl ester were dissolved in a mixture of 2.7 ml of trifluoroacetic acid and 0.3 ml of water. The mixture was allowed to stand at room temperature for one hour and was concentrated in a water-jet vacuum. For purification, the substance was chromatographed on Sephadex LH20 using a mixture of glacial acetic acid, n-butanol and water. The fractions containing the pure substance were concentrated. The residue was dissolved in water and the solution was freeze-dried. Yield: 30 mg of white solid (15%).

FAB-MS: 719.0 (M+H)⁺

### Example 19

((R,S)-4-(4-(Amino-imino-methyl)phenyl)-3-benzyl-4-methyl-2,5-dioxo-imidazolidin-1-yl)acetyl-L-N-methyl-aspartyl-L-phenylglycine

The compound of Example 19 was prepared analogously to Example 18.

FAB-MS: 643.2 (M+H)⁺

The compounds of Examples 20 to 25 were prepared analogously to Example 2.

### Example 20

((R,S)-4-(4-(Amino-imino-methyl)phenyl)-3,4-dibenzyl-2,5-dioxo-imidazolidin-1-yl)acetyl-L-aspartyl-L-phenylglycine

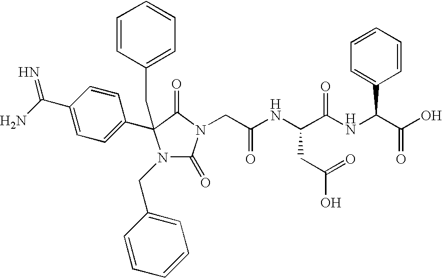

FAB-MS: 705.2 (M+H)⁺

### Example 21

((R,S)-4-(4-(Amino-imino-methyl)phenyl)-3-benzyl-4-methyl-2,5-dioxo-imidazolidin-1-yl)acetyl-L-aspartyl-L-valine

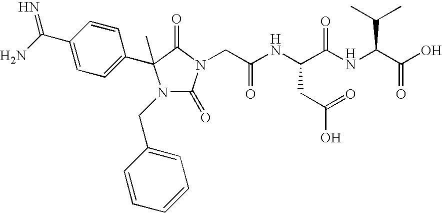

FAB-MS: 595.2 (M+H)⁺

### Example 22

((R,S)-4-(4-(Amino-imino-methyl)phenyl)-3-benzyl-4-methyl-2,5-dioxo-imidazolidin-1-yl)acetyl-L-aspartyl-L-leucine

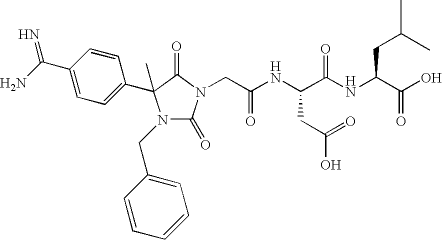

FAB-MS: 609.3 (M+H)⁺

### Example 23

((R,S)-4-(4-(Amino-imino-methyl)phenyl)-3-benzyl-4-methyl-2,5-dioxo-imidazolidin-1-yl)acetyl-L-aspartyl-L-serine

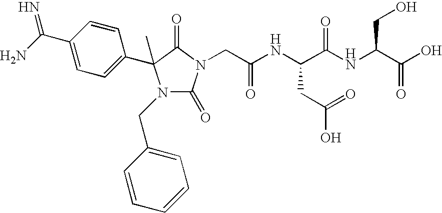

FAB-MS: 583.2 (M+H)⁺

### Example 24

((R,S)-4-(4-(Amino-imino-methyl)phenyl)-3-benzyl-4-methyl-2,5-dioxo-imidazolidin-1-yl)acetyl-L-aspartyl-L-phenylalanine

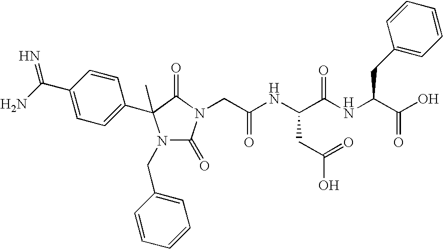

FAB-MS: 643.3 (M+H)⁺

### Example 25

((R,S)-4-(4-(Amino-imino-methyl)phenyl)-3-benzyl-4-methyl-2,5-dioxo-imidazolidin-1-yl)acetyl-L-aspartyl-L-phenylglycine methyl ester

FAB-MS: 643.2 (M+H)⁺

### Example 26

((R,S)-4-(4-(Amino-imino-methyl)phenyl)-3-benzyl-4-methyl-2,5-dioxoimidazolidin-1-yl)acetyl-L-aspartyl-(2-adamantylamide)

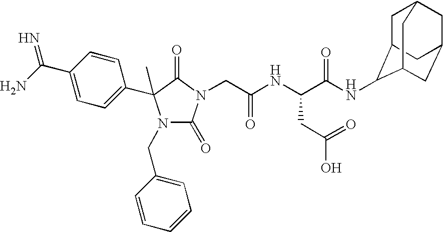

FAB-MS: 629.5 (M+H)⁺

### Example 27

((R,S)-4-(4-(Amino-imino-methyl)phenyl)-3-benzyl-4-methyl-2,5-dioxoimidazolidin-1-yl)acetyl-L-aspartyl-(1-adamantylamide)

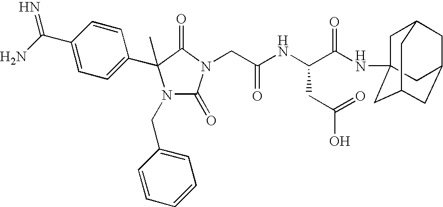

FAB-MS: 629.3 (M+H)⁺

### Example 28

((R,S)-4-(4-(Amino-imino-methyl)phenyl)-3-benzyl-4-methyl-2,5-dioxoimidazolidin-1-yl)acetyl-L-aspartyl-((1-adamantylmethyl)amide)

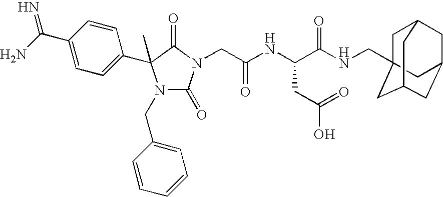

FAB-MS: 643.4 (M+H)⁺

The compounds of Examples 29 and 30 are diastereomers. One of the compounds of Examples 29 and 30 has the (S) configuration at the chiral center on C-4 of the imidazolidine ring, the other has the (R) configuration. The compounds were obtained from the compound of Example 4 by separation by means of preparative HPLC (acetonitrile/water/ammonium acetate (17:83:0.1)).

### Example 29

### Diastereomer I

((S or R)-4-(4-(Amino-imino-methyl)phenyl)-3-benzyl-4-methyl-2,5-dioxoimidazolidin-1-yl)acetyl-L-aspartyl-L-phenylglycine

FAB-MS: 628.3 (M+H)⁺

### Example 30

### Diastereomer II

((R or S)-4-(4-(Amino-imino-methyl)phenyl)-3-benzyl-4-methyl-2,5-dioxoimidazolidin-1-yl)acetyl-L-aspartyl-L-phenylglycine

FAB-MS: 628.3 (M+H)⁺

### Example 31

(R,S)-3-(((R,S)-4-(4-(Amino-imino-methyl)phenyl)-3,4-dimethyl-2,5-dioxoimidazolidin-1-yl)acetylamino)-3-phenylpropionic acid hydrochloride

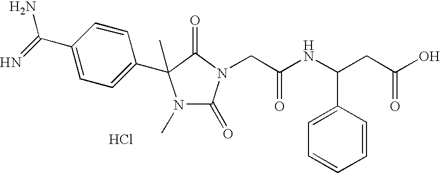

FAB-MS: 452 (M+H)⁺

### Example 32

(R,S)-3-(((R,S)-4-(4-(Amino-imino-methyl)phenyl)-3-ethyl-4-methyl-2,5-dioxoimidazolidin-1-yl)acetylamino)-3-phenylpropionic acid hydrochloride

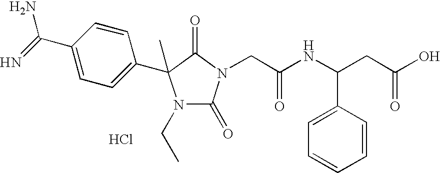

FAB-MS: 466 (M+H)⁺

### Example 33

(R,S)-3-(((R,S)-4-(4-(Amino-imino-methyl)phenyl)-3-benzyl-4-methyl-2,5-dioxoimidazolidin-1-yl)acetylamino)-3-phenylpropionic acid hydrochloride

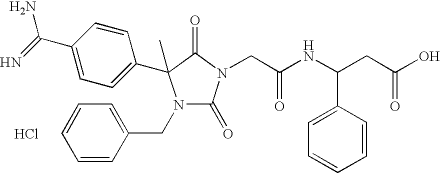

FAB-MS: 528.3 (M+H)⁺

### Example 34

3-(((R,S)-4-(4-(Amino-imino-methyl)phenyl)-3-benzyl-4-methyl-2,5-dioxoimidazolidin-1-yl)acetylamino)propionic acid hydrochloride

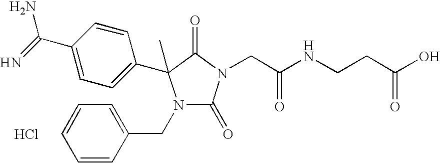

FAB-MS: 452.3 (M+H)⁺

### Example 35

((S)-4-(3-Aminopropyl)-3-ethyl-2,5-dioxoimidazolidin-1-yl)acetyl-L-aspartyl-L-phenylglycine

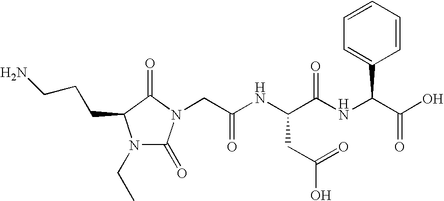

The compound was prepared analogously to steps a) to e) of Example 36, and the tert-butyl ester corresponding to the compound of Example 36e) was cleaved with trifluoroacetic acid.

FAB-MS: 492.6 (M+H)⁺

### Example 36

((S)-4-(3-Guanidinopropyl)-3-ethyl-2,5-dioxoimidazolidin-1-yl)acetyl-L-aspartyl-D-phenylglycine

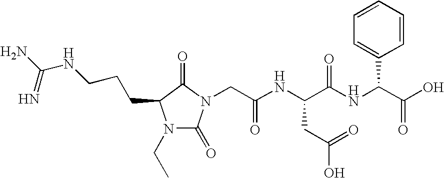

36a) Ethyl((S)-4-(3-benzyloxycarbonylaminopropyl)-2,5-dioxoimidazolidin-1-yl)acetate 52 g (185.7 mmol) of H-Orn(Z)-OCH₃ and 24.15 ml (185.7 mmol) of ethylmorpholine were dissolved in 500 ml of DMF and the solution was cooled to 0° C. 23.77 ml (185.7 mmol) of ethyl isocyanatoacetate were then added dropwise with stirring and the mixture was allowed to come to room temperature overnight. For working-up, the DMF was removed in vacuo and the residue was taken up in 500 ml of ethyl acetate. The ethyl acetate solution was washed several times with water, the ethyl acetate phase was cooled to 0° C. overnight and the precipitated product was filtered off. It was then recrystallized again from ethyl acetate.

Yield: 55.4 g (79%).

CI-MS: 378 (M+H)⁺

36b) Ethyl((S)-4-(3-benzyloxycarbonylaminopropyl)-3-ethyl-2,5-dioxo-imidazolidin-1-yl)acetate

6.74 g (17.8 mmol) of ethyl((S)-4-(3-benzyloxycarbonylaminopropyl)-2,5-dioxoimidazolidin-1-yl)acetate were cooled to 0° C. in 40 ml of DMF. 0.49 g (20 mmol) of sodium hydride was then added in portions. The mixture was subsequently treated with 2.18 g (20 mmol) of ethyl bromide and allowed to come to room temperature overnight. After completion of the reaction, the solvent was removed in vacuo and the crude product was separated by chromatography on silica gel using methylene chloride/methanol/acetic acid/water (99:1:0.1:0.1). Yield: 5.4 g (75%).

ES-MS: 406 (M+H)⁺

36c) ((S)-4-(3-Benzyloxycarbonylaminopropyl)-3-ethyl-2,5-dioxoimidazolidin-1-yl)acetic acid

2.025 g (5 mmol) of ethyl((S)-4-(3-benzyloxycarbonylaminopropyl)-3-ethyl-2,5-dioxoimidazolidin-1-yl)acetate were dissolved in 15 ml of ethanol and treated with 50 ml of a 0.1 N LiOH solution. The mixture was stirred at room temperature for 4 days. After completion of the reaction, it was treated with 200 ml of water and adjusted to pH 3 using citric acid. The aqueous phase was extracted with ethyl acetate, and the organic phase was washed several times with water and concentrated. The residue was chromatographed on silica gel using methylene chloride/methanol (8:3).

Yield: 810 mg (40%).

ES-MS: 378.3 (M+H)⁺

36d) ((S)-4-(3-Benzyloxycarbonylaminopropyl)-3-ethyl-2,5-dioxo-imidazolidin-1-yl)acetyl-L-aspartyl-D-phenylglycine di-tert-butyl ester

810 mg (2.15 mmol) of ((S)-4-(3-benzyloxycarbonylaminopropyl)-3-ethyl-2,5-dioxoimidazolidin-1-yl)acetic acid were treated in 10 ml of DMF with 87 mg (2.36 mmol) of DCC and 290 mg (2.15 mmol) of HOBt and the mixture was stirred for 30 minutes. 928 mg (2.15 mmol) of H-Asp(Obu^{t})-d-Phg-Obu^{t} and 280 μl (2.15 mmol) of N-ethylmorpholine were then added. The mixture was allowed to react overnight. After completion of the reaction, the solvent was removed in vacuo, the residue was taken up in methylene chloride and the precipitated dicyclohexylurea was filtered off. The solution was concentrated and the residue was chromatographed on silica gel using methylene chloride/methanol/acetic acid/water (97:3:0.1:0.1). Yield: 1.5 g (94%).

ES-MS: 738.4 (M+H)⁺

36e) ((S)-4-(3-Aminopropyl)-3-ethyl-2,5-dioxoimidazolidin-1-yl)acetyl-L-aspartyl-D-phenylglycine di-tert-butyl ester

1.5 g (2.03 mmol) of ((S)-4-(3-benzyloxycarbonylaminopropyl)-3-ethyl-2,5-dioxoimidazolidin-1-yl)acetyl-L-aspartyl-D-phenylglycine di-tert-butyl ester were dissolved in 70 ml of methanol, treated with 0.5 g of 10% strength palladium/active carbon catalyst and hydrogenated. After completion of the reaction, the catalyst was filtered off with suction, the filtrate was concentrated and the residue was dried in vacuo. Yield: 1.2 g (92%).

MS: 604.3 (M+H)⁺

36f) ((S)-4-(3-Guanidinopropyl)-3-ethyl-2,5-dioxoimidazolidin-1-yl)acetyl-L-aspartyl-D-phenylglycine di-tert-butyl ester

1 g (1.56 mmol) of ((S)-4-(3-aminopropyl)-3-ethyl-2,5-dioxoimidazolidin-1-yl)acetyl-L-aspartyl-D-phenylglycine di-tert-butyl ester was dissolved in 17 ml of DMF. 0.228 g (1.56 mmol) of 1H-pyrazole-1-carboxamidine hydrochloride and 0.8 ml (4.68 mmol) of diisopropylethylamine were added to the solution and it was allowed to react overnight. After completion of the reaction, the solvent was removed in vacuo and the residue was purified by chromatography on silica gel using methylene chloride/methanol/acetic acid/water (95:5:0.5:0.5). Yield: 0.68 g (68%).

ES-MS: 646.4 (M+H)⁺

36g) ((S)-4-(3-Guanidinopropyl)-3-ethyl-2,5-dioxoimidazolidin-1-yl)acetyl-L-aspartyl-D-phenylglycine

0.68 g (1.05 mmol) of ((S)-4-(3-guanidinopropyl)-3-ethyl-2,5-dioxo-imidazolidin-1-yl)acetyl-L-aspartyl-D-phenylglycine di-tert-butyl ester was dissolved in 10 ml of a mixture of trifluoroacetic acid and water (95:5). After 30 minutes, the reaction solution was concentrated in vacuo and the residue was taken up in 100 ml of water. It was then converted into the acetic acid salt using Amberlite IRA 93/45 and purified by chromatography on Sephadex G25 using 1 M acetic acid. Yield: 0.181 g (32%).

FAB-MS: 534.3 (M+H)⁺

### Example 37

((S)-4-(3-Aminopropyl)-3-benzyl-2,5-dioxoimidazolidin-1-yl)acetyl-L-aspartyl-L-phenylglycine

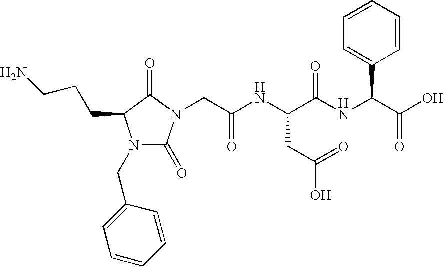

The compound of Example 37 was prepared analogously to Example 35.

FAB-MS: 553.6 (M+H)⁺

The compounds of Examples 38 to 40 were prepared analogously to Example 36.

### Example 38

((S)-4-(3-Guanidinopropyl)-3-benzyl-2,5-dioxoimidazolidin-1-yl)acetyl-L-aspartyl-L-phenylglycine

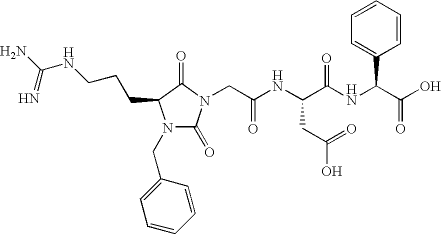

FAB-MS: 596.4 (M+H)⁺

### Example 39

((S)-4-(3-Guarndinopropyl)-3-benzyl-2,5-dioxoimidazolidin-1-yl)acetyl-D-aspartyl-L-phenylglycine-L-tyrosine hydrochloride

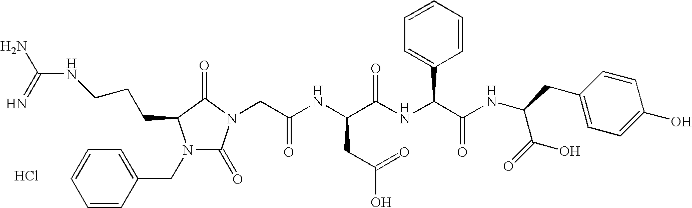

FAB-MS: 792.4 (M+H)⁺

### Example 40

((S)-4-(3-Guanidinopropyl)-3-benzyl-2,5-dioxoimidazolidin-1-yl)acetyl-D-glutamyl-L-phenylglycine-L-tyrosine hydrochloride

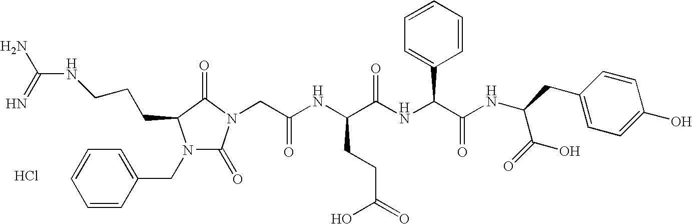

FAB-MS: 806.4 (M+H)⁺

### Example 41

((S)-4-(4-(Amino-imino-methyl)phenyl)-3-((2-naphthyl)methyl)-4-methyl-2,5-dioxoimidazolidin-1-yl)acetyl-L-aspartyl-L-phenylglycine

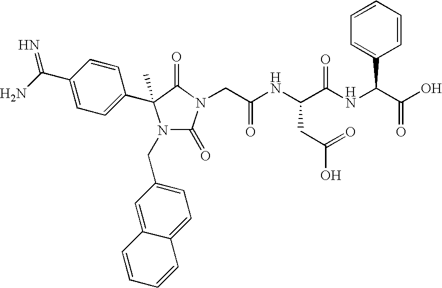

The compound was prepared by separation of ((R,S)-4-(4-(amino-imino-methyl)phenyl)-3-((2-naphthyl)methyl)-4-methyl-2,5-dioxoimidazolidin-1-yl)acetyl-L-aspartyl-L-phenylglycine (see Example 9) by means of MPLC on silica gel.

ES(+)-MS: 679.3 (M+H)⁺

### Example 42

(R,S)-3-(((R,S)-4-(4-(Amino-imino-methyl)phenyl)-3-benzyl-4-methyl-2,5-dioxoimidazolidin-1-yl)acetylamino)-2-(4-methoxybenzyl)propionic acid

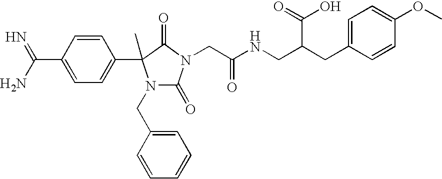

ES(+)-MS: 572.3 (M+H)⁺

### Example 43

(R,S)-3-(((R,S)-4-(4-(Amino-imino-methyl)phenyl)-3-benzyl-4-methyl-2,5-dioxoimidazolidin-1-yl)acetylamino)-2-phenylpropionic acid

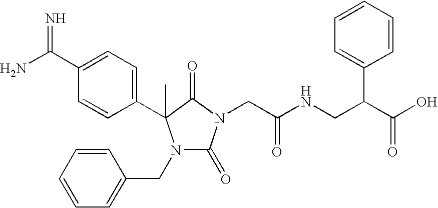

ES(+)-MS: 528.3 (M+H)⁺

### Example 44

(R,S)-3-(((R,S)-4-(4-(Amino-imino-methyl)phenyl)-3-benzyl-4-methyl-2,5-dioxoimidazolidin-1-yl)acetylamino)-2-((1-naphthyl)methyl)propionic acid

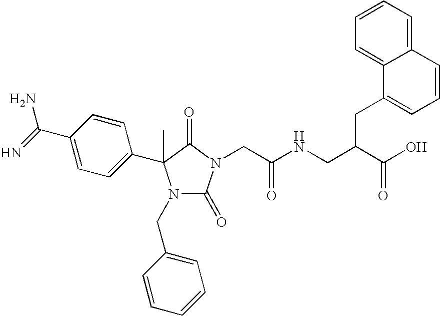

ES(+)-MS: 592.4 (M+H)⁺

### Example 45

(R,S)-3-(((R,S)-4-(4-(Amino-imino-methyl)phenyl)-3-benzyl-4-methyl-2,5-dioxoimidazolidin-1-yl)acetylamino)-2-(4-tert-butyl-benzyl)propionic acid

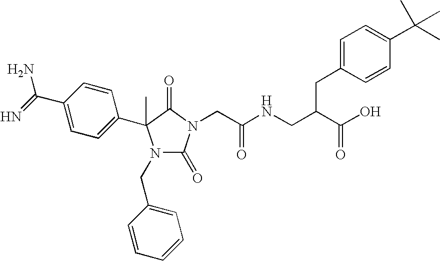

ES(+)-MS: 598.4 (M+H)⁺

### Example 46

(S)-3-(((R,S)-4-(4-(Amino-imino-methyl)phenyl)-3-benzyl-4-methyl-2,5-dioxoimidazolidin-1-yl)acetylamino)-2-benzyloxycarbonylaminopropionic acid

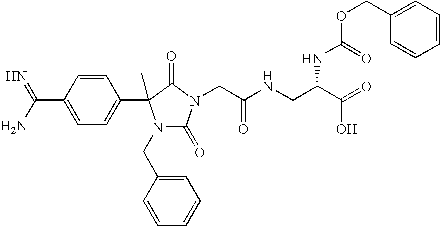

46a) tert-Butyl(S)-3-amino-2-benzyloxycarbonylaminopropionate

10 g (42 mmol) of (S)-3-amino-2-benzyloxycarbonylaminopropionic acid were shaken in an autoclave under an N₂ pressure of 20 atm for 3 days in a mixture of 100 ml of dioxane, 100 ml of isobutylene and 8 ml of conc. H₂SO₄. Excess isobutylene was blown out and 150 ml of diethyl ether and 150 ml of saturated NaHCO₃ solution were added to the remaining solution. The phases were separated and the aqueous phase was extracted 2× using 100 ml of diethyl ether each time. The combined organic phases were washed with 2×100 ml of water and dried over Na₂SO₄. After removing the solvent in vacuo, 9.58 g (78%) of product were obtained as a pale yellow oil.

46b) tert-Butyl(S)-3-(((R,S)-4-(4-(amino-imino-methyl)phenyl)-3-benzyl-4-methyl-2,5-dioxoimidazolidin-1-yl)acetylamino)-2-benzyloxycarbonylaminopropionate

208 mg (0.5 mmol) of ((R,S)-4-(4-(amino-imino-methyl)phenyl)-3-benzyl-4-methyl-2,5-dioxoimidazolidin-1-yl)acetic acid hydrochloride (prepared analogously to Example 2a using benzyl bromide instead of methyl iodide and consequent reactions, see Examples 2b-2d) and 81.5 mg (0.5 mmol) of HOOBt were suspended in 5 ml of DMF and treated at 0° C. with 110 mg (0.55 mmol) of DCC. The mixture was stirred at 0° C. for 1 h and at RT for 1 h and 147 mg (0.5 mmol) tert-butyl(S)-3-amino-2-benzyloxycarbonyl-aminopropionate were then added, and the mixture was stirred at room temperature for 2 h and allowed to stand at room temperature overnight. The solvent was removed in vacuo and the residue was chromatographed on silica gel using dichloromethane/methanol/glacial acetic acid/water (9:1:0.1:0.1). After concentrating and freeze-drying, 225 mg (69%) of tert-butyl(S)-3-(((R,S)-4-(4-(amino-imino-methyl)phenyl)-3-benzyl-4-methyl-2,5-dioxoimidazolidin-1-yl)acetylamino)-2-benzyloxycarbonylaminopropionate were obtained as a colorless solid.

46c) (S)-3-(((R,S)-4-(4-(Amino-imino-methyl)phenyl)-3-benzyl-4-methyl-2,5-dioxoimidazolidin-1-yl)acetylamino)-2-benzyloxycarbonylaminopropionic acid

220 mg (0.33 mmol) of tert-butyl(S)-3-(((R,S)-4-(4-(amino-imino-methyl)phenyl)-3-benzyl-4-methyl-2,5-dioxoimidazolidin-1-yl)acetylamino)-2-benzyloxycarbonylaminopropionate were allowed to stand at room temperature for 1 h in 5 ml of 90% strength trifluoroacetic acid. After concentrating in vacuo, the residue was chromatographed on silica gel using dichloromethane/methanol/glacial acetic acid/water (8:2:0.2:0.2). After concentrating the product fractions and freeze-drying, 155 mg (78%) of (S)-3-(((R,S)-4-(4-(amino-imino-methyl)phenyl)-3-benzyl-4-methyl-2,5-dioxoimidazolidin-1-yl)acetylamino)-2-benzyloxycarbonylaminopropionic acid were obtained as a colorless solid.

FAB-MS: 601.3 (M+H)

### Example 47

(R)-3-(((R,S)-4-(4-(Amino-imino-methyl)phenyl)-3-((2-naphthyl)methyl)-4-methyl-2,5-dioxoimidazolidin-1-yl)acetylamino)-2-benzyloxycarbonylaminopropionic acid

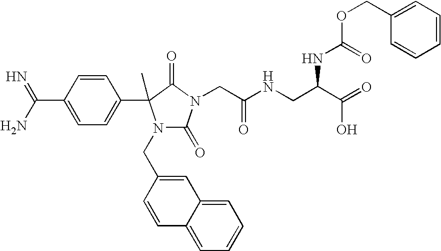

The synthesis was carried out analogously to Example 46 using tert-butyl(R)-3-amino-2-benzyloxycarbonylaminopropionate instead of the (S) isomer and 2-bromomethylnaphthalene instead of benzyl bromide.

ES(+)-MS: 651.3 (M+H)⁺

### Example 48

(S)-3-(((R,S)-4-(4-(Amino-imino-methyl)phenyl)-3-benzyl-4-methyl-2,5-dioxoimidazolidin-1-yl)acetyl-N-methylamino)-2-benzyloxycarbonylaminopropionic acid

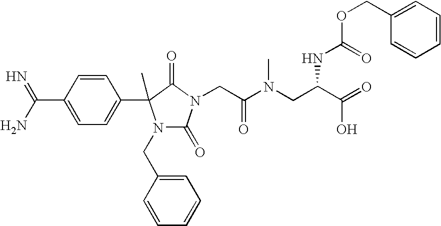

The synthesis was carried out analogously to Example 46 using tert-butyl(S)-2-benzyloxycarbonylamino-3-(N-methylamino)propionate (prepared from tert-butyl(S)-3-amino-2-benzyloxycarbonylaminopropionate analogously to S. C. Miller, T. S. Scanlan, J. Am. Chem. Soc. 1997, 119, 2301).

ES(+)-MS: 615.3 (M+H)⁺

### Example 49

((R,S)-4-(4-(Amino-imino-methyl)phenyl)-3-benzyl-4-methyl-2,5-dioxoimidazolidin-1-yl)acetyl-L-aspartic acid

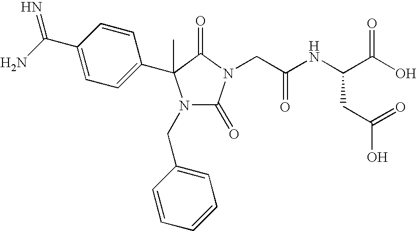

ES(+)-MS: 496.1 (M+H)⁺

### Example 50

((R,S)-2((S)-4-(4-(Amino-imino-methyl)phenyl)-3-benzyl-4-methyl-2,5-dioxoimidazolidin-1-yl)-2-(2-methylpropyl)acetyl)-L-aspartyl-L-phenylglycine

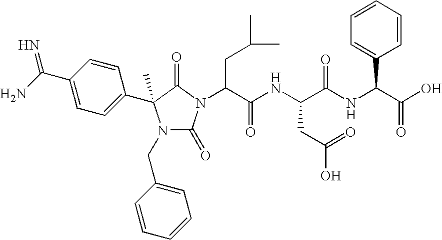

50a) (S)-4-(4-Cyanophenyl)-4-methyl-2,5-dioxoimidazolidine (50.1)

6 g (22.2 mmol) of (S)-4-(4-bromophenyl)-4-methyl-2,5-dioxoimidazolidine and 9 g (100.2 mmol) of copper(I) cyanide were heated under reflux for 5 h in 57 ml of DMF. After cooling to room temperature, the mixture was treated with water and ethyl acetate and adjusted to a pH of 2 using 2 N hydrochloric acid with ice-cooling. After filtration, the water phase was extracted 2× with ethyl acetate. The combined organic phases were washed with water and saturated sodium chloride solution, dried over sodium sulfate and concentrated in vacuo after filtration. The crude product was chromatographed on silica gel using dichloromethane/methanol (95:5). After concentrating the product fractions, 2.74 g (57%) of (50.1) were obtained.

50b) tert-Butyl(R,S)-2-((S)-4-(4-cyanophenyl)-4-methyl-2,5-dioxoimidazolidin-1-yl)-4-methylpentanoate (50.2)

128 mg (5.35 mmol) of sodium hydride were added to a solution of 1 g (4.64 mmol) of (50.1) in 15 ml of absolute DMF under argon, the mixture was stirred at room temperature for 2 h, 1.23 g (4.9 mmol) of tert-butyl D,L-2-bromo-4-methylpentanoate (50.7) were added (preparation see 50f) and the mixture was stirred at room temperature for 4 h. After addition of a further 40 mg of sodium hydride, the mixture was allowed to stand at room temperature for 3 days, the solvent was removed in vacuo and the residue was partitioned between ethyl acetate and water. A pH of 4 was set with saturated KHSO₄/K₂SO₄ solution, the phases were separated and the aqueous phase was extracted 2× using ethyl acetate. The combined organic phases were dried over sodium sulfate, the drying agent was filtered off and the filtrate was concentrated in vacuo. The residue was filtered through silica gel using heptane/ethyl acetate (2:1, then 1:2). After concentrating the product fractions, 666 mg (37%) of (50.2) were obtained.

50c) tert-Butyl(R,S)-2-((S)-4-(4-cyanophenyl)-3-benzyl-4-methyl-2,5-dioxoimidazolidin-1-yl)-4-methylpentanoate (50.3)

74 mg (3.07 mmol) of sodium hydride were added at 0° C. to a solution of 990 mg (2.56 mmol) of (50.2) in absolute DMF under argon at 0° C., the mixture was stirred at room temperature for 1 h, 334 μl (2.81 mmol) of benzyl bromide were added and the mixture was stirred at room temperature for 1.5 h. The solvent was removed in vacuo, the residue was partitioned between water and ethyl acetate and, after phase separation, the water phase was extracted with ethyl acetate. The combined organic phases were dried over sodium sulfate, the drying agent was filtered off and the filtrate was concentrated in vacuo. 1.22 g (100%) of (50.3) were obtained.

50d) tert-Butyl(R,S)-2-(((S)-4-(amino-hydroximino-methyl)phenyl)-3-benzyl-4-methyl-2,5-dioxoimidazolidin-1-yl)-4-methylpentanoate (50.4)

353 mg (6.08 mmol) of hydroxylammonium chloride and 1.05 ml (7.62 mmol) of triethylamine were added to a solution of 1.21 g (2.54 mmol) of (50.3) in 30 ml of ethanol and the mixture was heated under reflux for 2 h. The solvent was removed in vacuo, the residue was taken up in water/ethyl acetate and, after phase separation, the water phase was extracted 2× with ethyl acetate. The combined organic phases were dried over sodium sulfate. The drying agent was filtered off and the solvent was removed in vacuo. 1.16 g (90%) of (50.4) were obtained.

50e) (R,S)-2-((S)-4-(4-(Amino-imino-methyl)phenyl)-3-benzyl-4-methyl-2,5-dioxoimidazolidin-1-yl)-4-methylpentanoic acid hydrochloride (50.5)

A solution of 850 mg (1.67 mmol) of (50.4) in 50 ml of acetic acid was hydrogenated over Raney nickel. After 2 h, the catalyst was filtered off, the filtrate was concentrated in vacuo, the residue was dissolved in 10% strength acetic acid, the solution was freeze-dried and the residue was dissolved in 10 ml of 90% strength trifluoroacetic acid. After 15 min at room temperature, the trifluoroacetic acid was removed in vacuo, and the residue was concentrated 2× with toluene, treated with 0.5 N hydrochloric acid and freeze-dried. 700 mg (87%) of (50.5) were obtained.

50f) ((R,S)-2-((S)-4-(4-(Amino-imino-methyl)phenyl)-3-benzyl-4-methyl-2,5-dioxo-imidazolidin-1-yl)-2-(2-methylpropyl)acetyl)-L-aspartyl-L-phenylglycine (50.6)

138 mg (0.422 mmol) of TOTU (O-[cyano(ethoxycarbonyl)methylene-amino]-1,1,3,3-tetramethyluronium tetrafluoroborate) and 216 μl (1.26 mmol) of diisopropylethylamine were added to a solution of 200 mg (0.422 mmol) of (50.5) and 175 mg (0.422 mmol) of H-Asp(O^{t}Bu)-Phg-O^{t}Bu hydrochloride in 20 ml of absolute DMF. After stirring at room temperature for 2 h, the reaction mixture was concentrated in vacuo, the residue was taken up in ethyl acetate and the organic phase was washed 2× with saturated NaHCO₃ solution and water. After drying over sodium sulfate, filtering and concentrating the filtrate in vacuo, 320 mg of crude product were obtained, which was chromatographed on silica gel using dichloromethane/methanol/glacial acetic acid/water (9:1:0.1:0.1). After concentrating the product fractions, the residue was dissolved in 5 ml of 90% strength trifluoroacetic acid, after 15 min at room temperature the trifluoroacetic acid was removed in vacuo, the residue thus obtained was concentrated 2× with toluene and the residue was finally dissolved in 20% strength acetic acid and freeze-dried. 132 mg (46%) of (50.6) were obtained.

ES(+)-MS: 685.4 (M+H)⁺

50g) Synthesis of tert-butyl D,L-2-bromo-4-methylpentanoate (50.7)

1.96 ml of concentrated sulfuric acid and 0.515 ml of oleum (20% strength) were added to a solution of 2.5 g (12.8 mmol) of D,L-2-bromo-4-methyl-pentanoic acid in 80 ml of chloroform and 80 ml of tert-butyl acetate and the mixture was stirred at room temperature for 3 h. A pH of 4 was set by addition of 10% strength NaHCO₃ solution. The aqueous phase was separated off and extracted 2× with dichloromethane. The combined organic phases were dried over sodium sulfate. After filtration alid concentration of the filtrate in vacuo, 2.62 g (82%) of (50.7) were obtained.

### Examples 51 and 52

The compounds of Examples 51 and 52 are diastereomers. They were obtained by separation of the diastereomer mixture (50.6) from Example 50 by means of preparative HPLC (RP-18; eluent A/B=60:40; A=water/0.1% trifluoroacetic acid; B=80% acetonitrile/20% water/0.1% trifluoroacetic acid). One of the compounds of Examples 51 and 52 has the (R) configuration at the chiral center in the 2-(2-methylpropyl)acetyl unit, the other has the (S) configuration.

### Example 51

((R or S)-2-((S)-4-(4-(Amino-imino-methyl)phenyl)-3-benzyl-4-methyl-2,5-dioxo-imidazolidin-1-yl)-2-(2-methylpropyl)acetyl)-L-aspartyl-L-phenylglycine ES(+)-MS: 685.4 (M+H)⁺

### Example 52

((S or R)-2-((S)-4-(4-(Amino-imino-methyl)phenyl)-3-benzyl-4-methyl-2,5-dioxo-imidazolidin-1-yl)-2-(2-methylpropyl)acetyl)-L-aspartyl-L-phenylglycine

ES(+)-MS: 685.4 (M+H)⁺

### Example 53

(R,S)-3-((R,S)-2-((S)-4-(4-(Amino-imino-methyl)phenyl)-3-benzyl-4-methyl-2,5-dioxoimidazolidin-1-yl)-2-(2-methylpropyl)acetylamino)-3-(2,4-dimethoxyphenyl)propionic acid

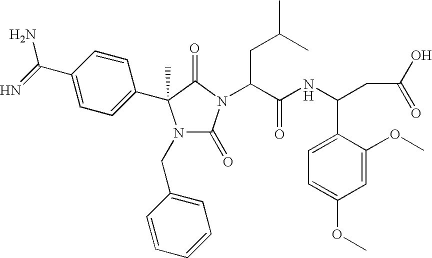

The compound was prepared by coupling of (R,S)-2-((S)-4-(4-(amino-imino-methyl)phenyl)-3-benzyl-4-methyl-2,5-dioxoimidazolidin-1-yl)-4-methylpentanoic acid hydrochloride (50.5) and tert-butyl(R,S)-3-amino-3-(2,4-dimethoxyphenyl)propionate hydrochloride and subsequent cleavage of the tert-butyl ester as described in Example 50.

ES(+)-MS: 644.4 (M+H)⁺

3-Substituted β-amino acids and β-amino acid esters, such as were employed in Example 53 and other examples, were obtained according to the following general synthesis procedure for the preparation of 3-substituted β-amino acids and 13-amino acid esters.

Racemic 3-substituted β-amino acids were prepared analogously to W. M. Radionow, E. A. Postovskaya, J. Am. Chem. Soc. 1929, 51, 841 (see also: Houben-Weyl, Methoden der Organischen Chemie [Methods of Organic Chemistry], Volume XI/2, Georg Thieme Verlag, Stuttgart, 1958, p. 497). The methyl esters or ethyl esters were prepared from these acids by methods known from the literature. The tert-butyl esters of the 3-substituted β-amino acids were prepared from these acids by first converting them into the β-benzyloxycarbonylamino acids. The tert-butyl esters were then prepared from these according to the following general synthesis procedure: 1.5 mmol of oxalyl chloride were added to 1 mmol of the β-benzyloxycarbonylaminocarboxylic acid in 13 ml of absolute dichloromethane. After stirring at room temperature for 4 h, the reaction mixture was concentrated and 6.5 ml of tert-butanol were added to the residue. The reaction mixture was stirred at room temperature for 1 h and concentrated in vacuo. The residue was taken up in ethyl acetate and extracted 2× with saturated NaHCO₃ solution and water. The organic phase was dried over sodium sulfate and, after filtration, the solvent was removed in vacuo. For preparation of the β-amino acid tert-butyl ester hydrochlorides, the benzyloxycarbonyl group was then removed hydrogenolytically by means of 10% Pd/C in methanol/HCl.

Enantiomerically pure 3-substituted β-amino acid esters were prepared analogously to S. G. Davis, O. Ichihara, Tetrahedron Asymmetry, 1991, 2, 183; S. G. Davis, N. M. Garrido, O. Ichihara, I. A. S. Walters, J. Chem. Soc., Chem. Commun., 1993, 1153; S. G. Davis, I. A. S. Walters, J. Chem. Soc., Perkin Trans. 1, 1994, 1129.

### Example 54

(R,S)-3-((R,S)-2-((S)-4-(4-(Amino-imino-methyl)phenyl)-3-benzyl-4-methyl-2,5-dioxoimidazolidin-1-yl)-2-(2-methylpropyl)acetylamino)-3-(3,4-methylendioxyphenyl)propionic acid

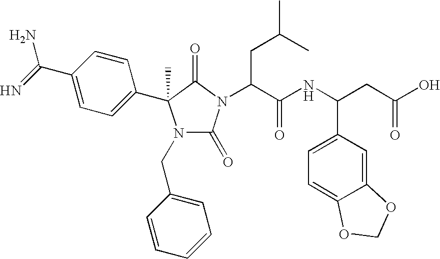

The compound was prepared by reaction of (50.5) with tert-butyl(R,S)-3-amino-3-(3,4-methylenedioxyphenyl)propionate hydrochloride and subsequent cleavage of the tert-butyl ester as described in Example 50.

ES(+)-MS: 628.4 (M+H)⁺

Examples 55 to 60 were carried out analogously to Example 54.

### Example 55

(R,S)-3-((R,S)-4-(4-(Amino-imino-methyl)phenyl)-3-benzyl-4-methyl-2,5-dioxoimidazolidin-1-yl)acetylamino)-3-(1-naphthyl)propionic acid

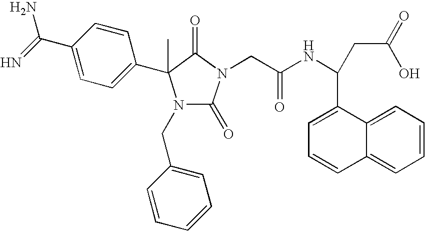

ES(+)-MS: 578.3 (M+H)⁺

### Example 56

(R,S)-3-(((R,S)-4-(4-(Amino-imino-methyl)phenyl)-3-benzyl-4-methyl-2,5-dioxoimidazolidin-1-yl)acetylamino)butyric acid

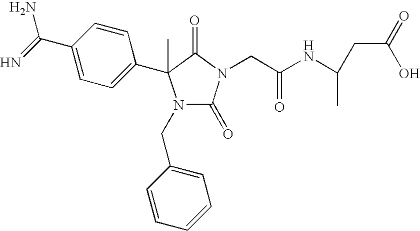

ES(+)-MS: 466.2 (M+H)⁺

### Example 57

(R,S)-3-(((R,S)-4-(4-(Amino-imino-methyl)phenyl)-3-benzyl-methyl-2,5-dioxoimidazolidin-1-yl)acetylamino)-3-(3,4-dimethoxyphenyl)propionic acid

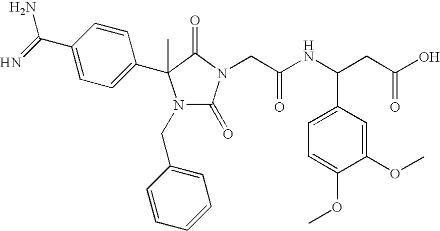

FAB-MS: 588.3 (M+H)⁺

### Example 58

(R,S)-3-(((R,S)-4-(4(Amino-imino-methyl)phenyl)-3-benzyl-4-methyl-2,5-dioxoimidazolidin-1-yl)acetylamino)-3-(3,4-ethylenedioxyphenyl)propionic acid

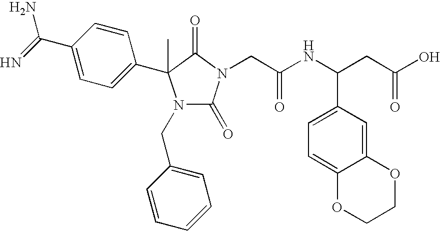

ES(+)-MS: 586.2 (M+H)⁺

### Example 59

(R,S)-3-(((R,S)-4-(4-(Amino-imino-methyl)phenyl)-3-benzyl-4-methyl-2,5-dioxoimidazolidin-1-yl)acetylamino)-3-(3,5-dimethoxyphenyl)propionic acid

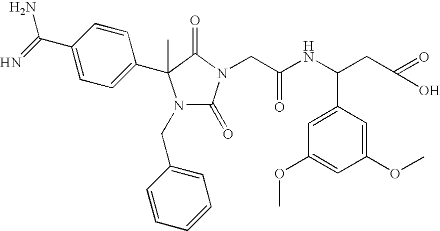

ES(+)-MS: 588.2 (M+H)⁺

### Example 60

(R,S)-3-(((R,S)-4-(4-(Amino-imino-methyl)phenyl)-3-benzyl-4-methyl-2,5-dioxoimidazolidin-1-yl)acetylamino)-3-(3-methoxyphenyl)propionic acid

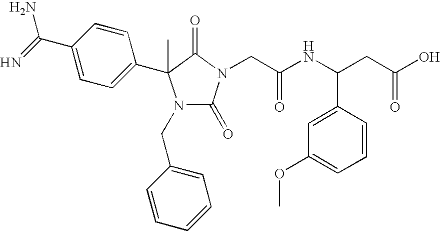

ES(+)-MS: 558.2 (M+H)⁺

### Example 61

(R,S)-3-(((R,S)-4-(4-(Amino-imino-methyl)phenyl)-3-benzyl-4-methyl-2,5-dioxoimidazolidin-1-yl)acetylamino)-3-(2-methoxyphenyl)propionic acid

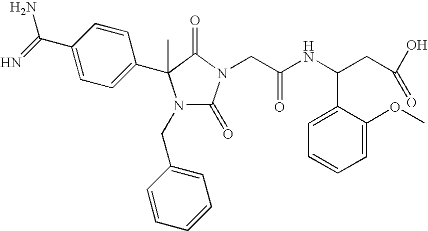

ES(+)-MS: 558.2 (M+H)⁺

### Example 62

((R,S)-2-((R,S)-4-(4-(Amino-imino-methyl)phenyl)-3-benzyl-4-methyl-2,5-dioxoimidazolidin-1-yl)-2-methylacetyl)-L-aspartyl-L-phenylglycine

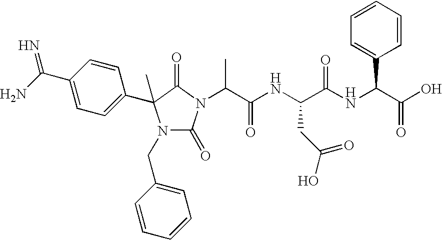

The compound was prepared analogously to Example 50 by reacting racemic (50.1) with ethyl(R,S)-2-bromopropionate and, before peptide coupling with H-Asp(O^{t}Bu)-Phg-O^{t}Bu×HCl, cleaving the ethyl ester with 6 N hydrochloric acid.

ES(+)-MS: 643.3 (M+H)⁺

### Example 63

(S)-4-(4-(Amino-imino-methyl)phenyl)-3-benzyl-4-methyl-2,5-dioxo-imidazolidin-1-yl)acetyl-L-aspartyl-L-phenylglycine

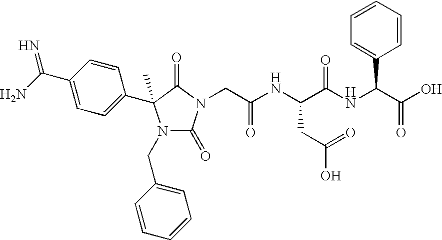

The compound was prepared analogously to Example 50 by reacting (50.1) with ethyl 2-bromoacetate and, before peptide coupling with H-Asp(O^{t}Bu)-Phg-O^{t}Bu×HCl, cleaving the ethyl ester with 6 N hydrochloric acid.

ES(+)-MS: 629.3 (M+H)⁺

### Example 64

(3-((R,S)-4-(4-(Amino-imino-methyl)phenyl)-3-benzyl-4-methyl-2,5-dioxoimidazolidin-1-yl)propionyl)-L-aspartyl-L-phenylglycine

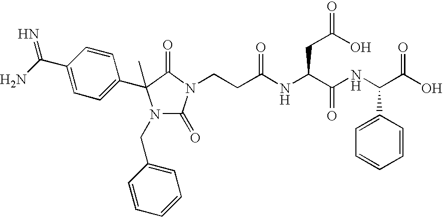

The compound was prepared analogously to Example 50 by reacting racemic (50.1) with ethyl 3-bromopropionate and, before peptide coupling with H-Asp(O^{t}Bu)-Phg-O^{t}Bu×HCl, cleaving the ethyl ester with 6 N hydrochloric acid.

ES(+)-MS: 643.3 (M+H)⁺

### Example 65

((R,S)-4-(4-(Amino-imino-methyl)phenyl)-3-benzyl-4-methyl-2,5-dioxoimidazolidin-1-yl)acetyl-L-aspartyl-L-N-methyl-phenylglycine

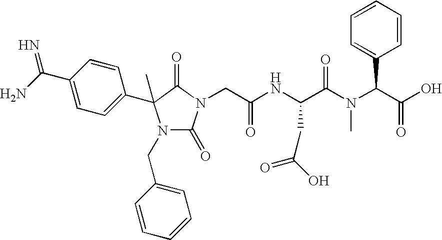

The compound was obtained analogously to Example 63, employing racemic (50.1) and

coupling with H-Asp(O^{t}Bu)-(N-methyl-Phg)-O^{t}Bu×HCl instead of H-Asp(O^{t}Bu)-Phg-O^{t}Bu×HCl.

ES(+)-MS: 643.3 (M+H)⁺

### Example 66

(R,S)-3-(((R,S)-4-(4-(Amino-imino-methyl)phenyl)-3-benzyl-4-methyl-2,5-dioxoimidazolidin-1-yl)acetyl-(N-methylamino))-3-phenylpropionic acid

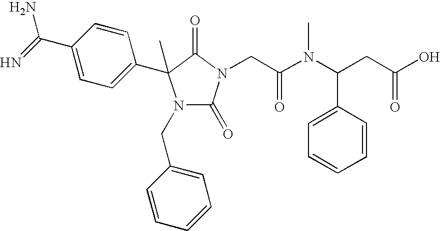

ES(+)-MS: 542.3 (M+H)⁺

### Example 67

(S)-3-((R,S)-4-(4-(Amino-imino-methyl)phenyl)-3-benzyl-4-methyl-2,5-dioxoimidazolidin-1-yl)acetylamino)-2-(4-trifluoromethylbenzyloxy-carbonylamino)propionic acid

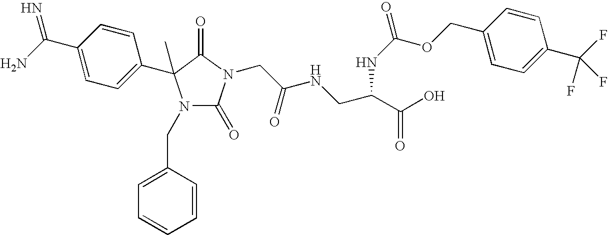

0.22 ml (1.29 mmol), of diisopropylethylamine and 204 mg (0.644 mmol) of N-(4-trifluoromethylbenzyloxycarbonyloxy)succimide were added to a solution of 300 mg (0.644 mmol) of (S)-3-(((R,S)-4-(4-(amino-imino-methyl)phenyl)-3-benzyl-4-methyl-2,5-dioxoimidazolidin-1-yl)acetylamino)-2-aminopropionic acid dihydrochloride (prepared from tert-butyl(S)-3-(((R,S)-4-(4-(amino-imino-methyl)phenyl)-3-benzyl-4-methyl-2,5-dioxoimidazolidin-1-yl)acetylamino)-2-benzyloxycarbonylaminopropionate by hydrogenolytic cleavage of the benzyloxycarbonyl group and cleavage of the tert-butyl ester with 6 N hydrochloric acid) and the mixture was stirred at room temperature for 4 h. The solvent was removed in vacuo and the residue was chromatographed on silica gel using dichloromethane/methanol/acetic acid/water (9:1:0.1:0.1) and methanol. The product fractions were concentrated and chromatographed on Sephadex LH20 using 40% strength acetic acid. 145 mg (37%) of the desired product were obtained after concentrating the product fractions.

ES(+)-MS: 669.3 (M+H)

### Example 68

((R,S)-4-(4-Aminomethylphenyl)-3-benzyl-4-methyl-2,5-dioxoimidazolidin 1-yl)acetyl-Laspartyl-L-phenylglycine

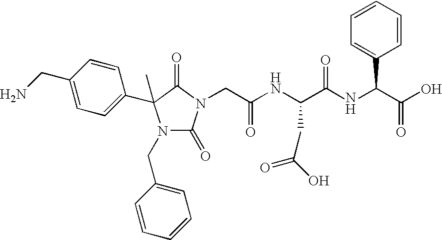

### Example 69

((R,S)-4-(4-Guanidinomethylphenyl)-3-benzyl-4-methyl-2,5-dioxo-imidazolidin-1-yl)acetyl-L-aspartyl-L-phenylglycine

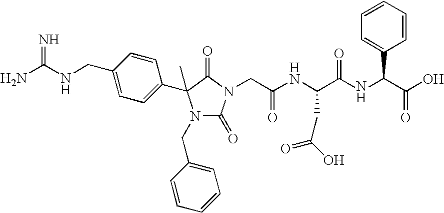

69a) Methyl((R,S)-4-(4-aminomethylphenyl)-3-benzyl-4-methyl-2,5-dioxo-imidazolidin-1-yl)acetate

7.55 g (20 mmol) of methyl((R,S)-4-(4-cyanophenyl)-3-benzyl-4-methyl-2,5-dioxoimidazolidin-1-yl)acetate (prepared from racemic 50.1 by reaction with methyl 2-bromoacetate and conversion of the reaction product with benzyl bromide analogously to Example 50) were hydrogenated at a hydrogen pressure of 3 atm for 7 h over 1.5 g of Pd/C in 80 ml of a mixture of ethanol and 50% strength acetic acid. The catalyst was filtered off and the residue was chromatographed on silica gel using dichloromethane/methanol (8:2). After concentrating the product fractions, 7.6 g (100%) of methyl((R,S)-4-(4-aminomethylphenyl)-3-benzyl-4-methyl-2,5-dioxo-imidazolidin-1-yl)acetate were obtained.

69b) ((R,S)-4-(4-Aminomethylphenyl)-3-benzyl-4-methyl-2,5-dioxo-imidazolidin-1-yl)acetic acid

3.7 g (9.7 mmol) of methyl((R,S)-4-(4-aminomethylphenyl)-3-benzyl-4-methyl-2,5-dioxoimidazolidin-1-yl)acetate were heated under reflux for 6 h in 80 ml of concentrated hydrochloric acid. The solution was concentrated in vacuo, the residue was taken up in water, the solution was filtered and the filtrate-was freeze-dried. 2.79 g (78%) of ((R,S)-4-(4-aminomethylphenyl)-3-benzyl-4-methyl-2,5-dioxoimidazolidin-1-yl)acetic acid were obtained.

69c) ((R,S)-4-(4-Guanidinomethylphenyl)-3-benzyl-4-methyl-2,5-dioxoimidazolidin-1-yl)acetic acid

807 mg (2 mmol) of ((R,S)-4-(4-aminomethylphenyl)-3-benzyl-4-methyl-2,5-dioxoimidazolidin-1-yl)acetic acid in 20 ml of absolute DMF were treated with 1.02 ml (6 mmol) of diisopropylethylamine and then with 220 mg (2 mmol) of 1H-pyrazole-1-carboxamidine×HCl. The reaction mixture was stirred at 50° C. for 5 h and then allowed to stand at room temperature overnight. 0.2 ml (0.4 mmol) of diisopropylethylamine and 44 mg (0.4 mmol) of 1H-pyrazole-1-carboxamidine×HCl were added again and the mixture was stirred at 50° C. for a further 6 h. The reaction mixture was concentrated in vacuo, the residue was triturated 2× with diethyl ether, the diethyl ether was decanted off and the residue was chromatographed on Sephadex LH 20 using water/butanol/acetic acid (43:4.3:3.5). 682 mg (83%) of ((R,S)-4-(4-guanidinomethylphenyl)-3-benzyl-4-methyl-2,5-dioxoimidazolidin-1-yl)acetic acid were obtained.

69d) ((R,S)-4-(4-Guanidinomethylphenyl)-3-benzyl-4-methyl-2,5-dioxoimidazolidin-1-yl)acetyl-L-aspartyl-L-phenylglycine

The compound was prepared by coupling of ((R,S)-4-(4-guanidinomethylphenyl)-3-benzyl-4-methyl-2,5-dioxoimidazolidin-1-yl)acetic acid with H-Asp(O^{t}Bu)-Phg-(O^{t}Bu)×HCl and subsequent cleavage of the tert-butyl ester analogously to Example 2.

ES(+)-MS: 658.3 (M+H)⁺

### Example 70

((R,S)-4-(4-Aminophenyl)-3-benzyl-4-methyl-2,5-dioxoimidazolidin-1-yl)acetyl-L-aspartyl-L-phenylglycine

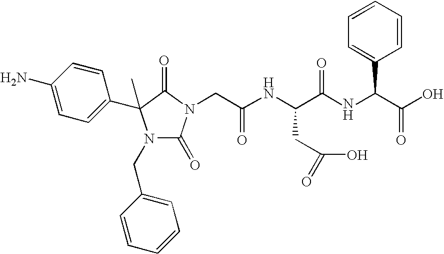

70a) ((R,S)-4-(4-Aminophenyl)-3-benzyl-4-methyl-2,5-dioxoimidazolidin-1-yl)acetic acid

A solution of 6.86 g (17.9 mmol) of ((R,S)-4-(4-nitrophenyl)-3-benzyl-4-methyl-2,5-dioxoimidazolidin-1-yl)acetic acid (prepared from 4-nitrophenyl methyl ketone analogously to the synthesis of ((R,S)-4-(4-cyano-phenyl)-3-benzyl-4-methyl-2,5-dioxoimidazolidin-1-yl)acetic acid from 4-cyanophenyl methyl ketone, cf. Example 1; for the introduction of the 3-benzyl group see Example 50) in 150 ml of methanol was hydrogenated over 10% Pd/C for 4 h. The catalyst was filtered off, the filtrate was concentrated in vacuo and the residue was triturated with diethyl ether and then filtered off with suction. 3.82 g (60%) of ((R,S)-4-(4-aminophenyl)-3-benzyl-4-methyl-2,5-dioxoimidazolidin-1-yl)acetic acid were obtained.

70b) ((R,S)-4-(4-Aminophenyl)-3-benzyl-4-methyl-2,5-dioxoimidazolidin-1-yl)acetyl-L-aspartyl-L-phenylglycine

The compound was prepared by coupling of ((R,S)-4-(4-aminophenyl)-3-benzyl-4-methyl-2,5-dioxoimidazolidin-1-yl)acetic acid with H-Asp(O^{t}Bu)-Phg-(O^{t}Bu)×HCl and subsequent cleavage of the tert-butyl ester analogously to Example 2, the residue from the trifluoroacetic acid cleavage being triturated with diethyl ether, filtered off with suction and dried in a high vacuum.

ES(+)-MS: 602.3 (M+H)⁺

### Example 71

((R,S)-4-(4-Guanidinophenyl)-3-benzyl-4-methyl-2,5-dioxoimidazolidin-1-yl)acetyl-Laspartyl-L-phenylglycine

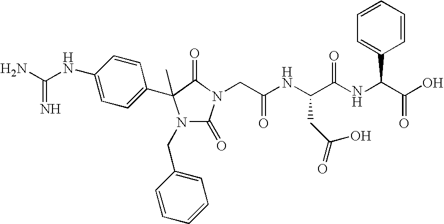

The compound was prepared from ((R,S)-4-(4-aminophenyl)-3-benzyl-4-methyl-2,5-dioxoimidazolidin-1-yl)acetic acid (see Example 70) after conversion of the amino group into the guanidino group using 1H-pyrazole-1-carboxamidine×HCl (as described in Example 69) and subsequent coupling with H-Asp(O^{t}Bu)-Phg-(O^{t}Bu)×HCl and cleavage of the tert-butyl ester analogously to Example 2, the residue from the trifluoroacetic acid cleavage only being triturated with diethyl ether, filtered off with suction and dried in a high vacuum.

ES(+)-MS: 644.3 (M+H)⁺

### Example 72

((R,S)-4-(4-Aminobenzyl)-3-benzyl-4-methyl-2,5-dioxoimidazolidin-1-yl)acetyl-L-aspartyl-L-phenylglycine

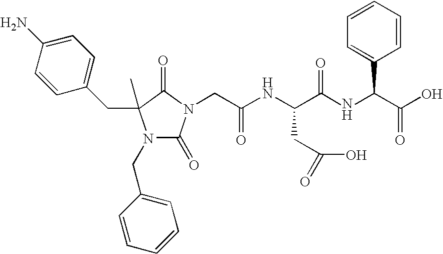

The compound was prepared by coupling ((R,S)-4-(4-aminobenzyl)-3-benzyl-4-methyl-2,5-dioxoimidazolidin-1-yl)acetic acid, which was synthesized analogously to Example 70 from ((R,S)-4-(4-nitrobenzyl)-3-benzyl-4-methyl-2,5-dioxoimidazolidin-1-yl)acetic acid (prepared from 4-nitrobenzyl methyl ketone analogously to the synthesis of ((R,S)-4-(4-cyanophenyl)-3-benzyl-4-methyl-2,5-dioxoimidazolidin-1-yl)acetic acid from 4-cyanophenyl methyl ketone, cf. Example 1; for the introduction of the 3-benzyl group see Example 50), with H-Asp(O^{t}Bu)-Phg-(O^{t}Bu)×HCl analogously to Example 2 and, after cleavage of the tert-butyl esters with 90% strength trifluoroacetic acid, chromatographing the residue on Sephadex LH using water/butanol/acetic acid (43:4.3:3.5).

ES(+)-MS: 616.3 (M+H)⁺

### Example 73

((R,S)-4-(4-Guanidinobenzyl)-3-benzyl-4-methyl-2,5-dioxoimidazolidin-1-yl)acetyl-L-aspartyl-L-phenylglycine

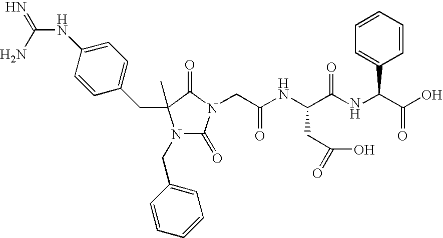

The compound was prepared from ((R,S)-4-(4-aminobenzyl)-3-benzyl-4-methyl-2,5-dioxoimidazolidin-1-yl)acetic acid (see Example 72) by conversion of the amino group into the guanidino group with 1H-pyrazole-1-carboxamidine×HCl (as described in Example 69), subsequent coupling with H-Asp(O^{t}Bu)-Phg-(O^{t}Bu)×HCl and cleavage of the tert-butyl esters analogously to Example 2, the residue from the trifluoroacetic acid cleavage being chromatographed on Sephadex LH 20 using water/butanol/acetic acid (43:4.3:3.5).

ES(+)-MS: 658.3 (M+H)⁺

Analogously to Example 67, the compounds of Examples 74 and 75 can also be prepared by, for example, reacting (S)-3-(((R,S)-4-(4-(amino-imino-methyl)phenyl)-3-benzyl-4-methyl-2,5-dioxoimidazolidin-1-yl)acetylamino)-2-aminopropionic acid dihydrochloride (see Example 67) with the corresponding carbonyl chlorides. tert-Butyl (S)-2-amino-3-tert-butoxycarbonylaminopropionate hydrochloride can also be used as a starting material for the preparation. Likewise, other benzyl carbamates having any desired substituents on the benzyl ring in the carbamate group can also be prepared analogously to Example 67.

### Example 74

(S)-3-((R,S)-2-((S)-4-(Amino-imino-methyl)phenyl)-3-benzyl-4-methyl-2,5-dioxoimidazolidin-1-yl)-2-(2-methylpropyl)acetylamino)-2-(2-methylpropyloxycarbonylamino)propionic acid

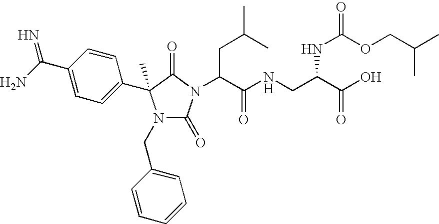

### Example 75

(S)-3-((R,S)-2-((S)-4-(4-(Amino-imino-methyl)phenyl)-3-benzyl-4-methyl-2,5-dioxoimidazolidin-1-yl)-2-(2-methylpropyl)acetylamino)-2-(2,2-dimethylpropyloxycarbonylamino)propionic acid

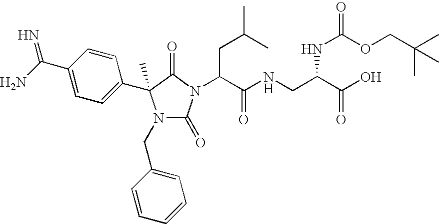

The compounds of Examples 77 to 79 were prepared by solid-phase synthesis according to the general procedure indicated in Example 76.

### Example 76

Solid-Phase Synthesis (General Procedure)

General

The syntheses on the polymeric support were carried out according to the synthesis sequence which is shown in Scheme 1. The radicals R⁵¹ to R⁵⁵ in Scheme 1 have the meaning of the radicals which are located in the position in the molecule concerned in formula I, or they can contain functional groups in protected form or in the form of precursors. R⁵¹ corresponds to the radicals R¹⁴ and R¹⁵, where functional groups present in these radicals can be present in protected form or in the form of precursors. R⁵² together with the CH group to which this radical is bonded, corresponds to the group B (R⁵² thus corresponds to a substituent on a methylene group representing B). R⁵³ corresponds to R¹³. R⁵⁴ corresponds to the group R¹-A, where functional groups present therein can be present in protected form or in the form of precursors, in particular, for example, in the present case an amidino group is present in the form of the cyano precursor. R^{54a} corresponds to the group R¹-A.R⁵⁵ corresponds to the group R⁰.

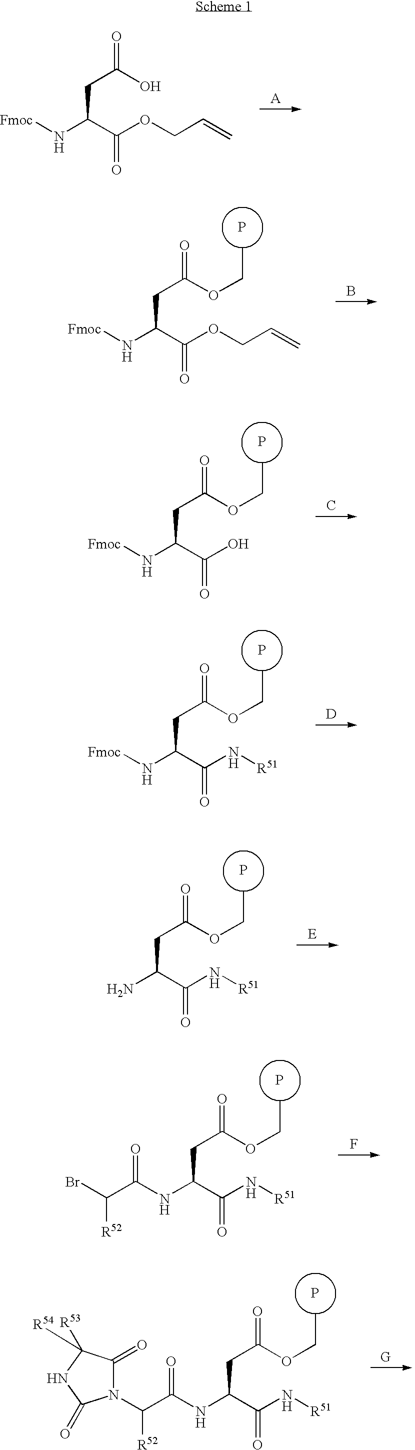

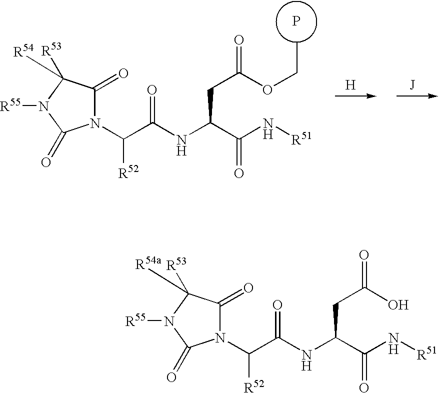

The synthesis of intermediates on a larger scale was carried out in special reaction vessels with frits inserted at the bottom of the reaction vessel; the synthesis of the compounds of the formula I was carried out in syringes or reaction blocks (Act 496, MultiSynTech). The syntheses on the resin were monitored by on bead analysis (FT-IR with ATR unit and MAS-NMR) and cleavage of an analytical sample from the resin (HPLC, MS. NMR).

Preparation of the aspartic acid building block FmocAsp(OH)Oallyl FmocAsp(OtBu)Oallyl (40 g, 88.7 mmol) was treated with 25 ml of trifluoroacetic acid and the mixture was stirred at room temperature for 30 min. The solvent was stripped off on a rotary evaporator. The residue was dried in vacuo. FmocAsp(OH)Oallyl was obtained as a yellow oil (33.9 g, 97%).

ES(+)-MS: 395.2 (M+H)⁺

Linkage to the Polymeric Support (Step A in Scheme 1)

40 g of Wang polystyrene resin (1.1 mmol/g; Bachem) were preswollen at room temperature with 20 ml of DMF for 5 min. After addition of a solution of 26.0 g (1.5 equivalents) of FmocAsp(OH)Oallyl and 34.3 g (1.5 equivalents) of 1-benzotriazolyloxytripyrrolidinophosphornum hexafluorophosphate (PyBOP) and 9.3 ml (1.5 equivalents) of diisopropylethylamine in 120 ml of DMF, the mixture was shaken at 40° C. for 10 h. After reaction was complete, the solution was filtered off with suction and the resin was washed with DMF (5×20 ml). After addition of a solution of acetic anhydride (10 ml) and diisopropylethylamine (9.3 ml, 1.5 equivalents) in 40 ml of DMF, the mixture was again shaken at room temperature for 30 min. The solution was filtered off with suction and the resin was washed in succession three times each with 40 ml of DMF, of methanol and of dichloromethane. The resin was then dried in vacuo. Determination of the loading by the Fmoc method showed a loading of 0.6 mmol/g.

Cleavage of the Allyl Group on the Polymeric Support (Step B)

The resin was preswollen at room temperature for 5 minin DMF under argon. After addition of tetrakis(triphenylphosphine)palladium and N-methylpyrrolidine (10 equivalents), the mixture was shaken at 40° C. for 6 h under argon. After reaction was complete, the solution was filtered off with suction and the resin was washed in succession three times each with DMF, methanol, toluene and dichloromethane and then dried.

Coupling with Amino Compounds on the Polymeric Support (Step C)

The loaded resin with a free carboxyl function was preswollen at room temperature for 5 min in DMF. After addition of a solution of HOBt (1.2 equivalents), TOTU (1.2 equivalents) and diisopropylethylamine (1.2 equivalents) in DMF, the mixture was shaken at room temperature for 30 min. The amino compound (1.2 equivalents) was added dissolved in DMF. The suspension was shaken at room temperature until reaction was complete (HPLC checking). After reaction was complete, the solution was filtered off with suction and the resin was washed in succession three times each with DMF, methanol, toluene and dichloromethane and then dried.

Cleavage of the Fmoc Protective Group (Step D)

For cleavage of the Fmoc protective group, the resin was preswollen at room temperature for 5 min in DMF. After addition of a solution of DMF/piperidine (1:1), the mixture was shaken at room temperature for 20 mm. The solution was filtered off with suction and the process was repeated. The cleavage of an analytical sample showed complete reaction according to HPLC/MS investigation. After complete reaction, the resin was washed three times with dichloromethane and employed directly in the coupling.

Coupling with α-halocarboxylic acids (Step E)

The symmetrical anhydrides were formed from α-halocarboxylic acids (5 equivalents) by 30-minute reaction with diisopropylcarbodiimide (DIC) (2.4 equivalents) in dichloromethane. After this time, 2 equivalents of diisopropylethylamine were added. The mixture was added to the resin and shaken at room temperature for 12 h. After reaction was complete, the solution was filtered off with suction and the resin was washed in succession three times each with DMF, toluene and dichloromethane and then immediately further reacted.

b) Coupling with Acid Halides

Instead of using acids and DIC, coupling can also be carried out using the acid halides. For this, the resin is preswollen at room temperature for 5 min with dichloromethane. The α-halocarboxylic acid halides (1.5 equivalents) are added dissolved in dichloromethane. After addition of a catalytic amount of 4-dimethylaminopyridine and diisopropylethylamine (1 equivalent), the mixture is shaken at room temperature for 8 h. After reaction is complete, the solution is filtered off with suction and the resin is washed in succession three times each with DMF, toluene and dichloromethane and then immediately further reacted.

Coupling of the α-haloacyl Compounds with Hydantoins (Step F)

The 4-cyanophenylhydantoins (2 equivalents) were activated in DMF with diazabicycloundecene (DBU) (2 equivalents) at room temperature. The activated solution was added after 15 min to the resin preswollen in DMF for 5 min. The mixture was shaken at room temperature for 8 h. After reaction was complete, the solution was filtered off with suction and the resin was washed in succession three times each with DMF, methanol, toluene and dichloromethane and then dried.

N-Alkylation of the Hydantoin on the Polymeric Support (Step G)

The resin was preswollen at room temperature for 5 min in DMF. After addition of cesium carbonate (3 equivalents), the mixture was shaken at room temperature for 30 min. After addition of the alkylating agent (bromide or iodide), the mixture was shaken at 50° C. for 6 h. After reaction was complete, the solution was filtered off with suction and the resin was washed in succession three times each with DMF, methanol/water/DMF (1.5:1.5:7), DMF, toluene and dichloromethane and then dried.

Instead of using cesium carbonate, the alkylation can also be carried out using phosphazenes. For this, the resin is preswollen at room temperature for 5 min in DMF. After addition of N′″-tert-butyl-N,N,N′,N′,N″,N″-hexamethylphosphorimidic triamide (phosphazene base P1-t-Bu) (3 equivalents), the mixture is shaken at room temperature for 30 min. After addition of the alkylating agent (bromide or iodide), the mixture is shaken at room temperature for 4 h. After reaction was complete, the solution is filtered off with suction and the resin is washed in succession three times each with DMF, toluene and dichloromethane and then dried.

Preparation of the amidino group from the cyano group on the polymeric support (step H) The resin was shaken at room temperature for 12 h with a saturated solution of hydrogen sulfide in pyridine/triethylamine (2:1). The solution was filtered off with suction and the resin was washed in succession three times each with methanol, DMF, toluene and dichloromethane. After addition of a 20% strength solution of methyl iodide in acetone/toluene (4:1), it was shaken at room temperature for a further 12 h. The solution was filtered off with suction and the resin was washed three times in succession in each case with acetone/toluene (4:1), DMF, methanol and methanol/toluene (4:1). After addition of ammonium acetate (10 equivalents) in methanol/toluene/acetic acid (80:16:4), the mixture was shaken at 50° C. for 3 h. After reaction was complete, the solution was filtered off with suction and the resin was washed in succession three times each with DMF, methanol, toluene and dichloromethane and then dried.

Cleavage from the Resin (Step J)

For removal of the compound from the resin, a mixture of trifluoroacetic acid/dichloromethane (1:1) was added to the resin. The suspension was shaken for 1 h. The resin was filtered off. The remaining solution was concentrated in vacuo. The residue was purified by silica gel chromatography (dichloromethane and ethyl acetate).

### Example 77

((R,S)-4-(4-(Amino-imino-methyl)phenyl)-3-((2-naphtyl)methyl)-4-methyl-2,5-dioxoimidazolidin-1-yl)acetyl-L-aspartyl-L-valine

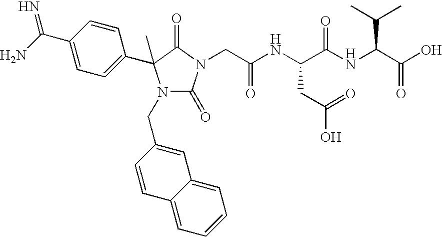

ES(+)-MS: 645.7 (M+H)⁺

### Example 78

((R,S)-4-(4-(Amino-imino-methyl)phenyl)-3-((2-naphthyl)methyl)₄-methyl-2,5-dioxoimldazolidin-1-yl)acetyl-L-aspartyl-L-serine methyl ester

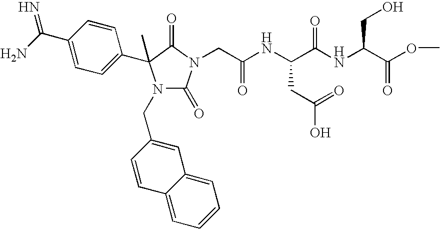

ES(+)-MS: 647.7 (M+H)⁺

### Example 79

((R,S)-4-(4-(Amino-imino-methyl)phenyl)-3-benzyl-4-methyl-2,5-dioxolmidazolidin-1-yl)acetyl-L-aspartyl-L-isoleucine

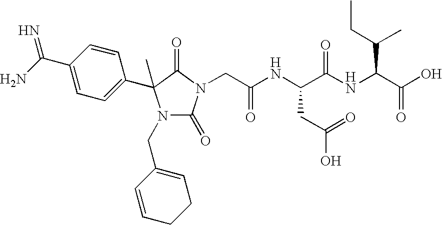

ES(+)-MS: 609.7 (M+H)⁺

Investigation of the Biological Activity

As a test method for the activity of the compounds of the formula I on the interaction between VCAM-1 and VLA-4, an assay which is specific for this interaction is used. The cellular binding components, i.e. the VLA-4 integrins, are offered in their natural form as surface molecules on human U937 cells (ATCC CRL 1593) which belong to the group of leucocytes. As specific binding components, recombinant soluble fusion proteins prepared by genetic engineering and consisting of the extracytoplasmic domains of human VCAM-1 and the constant region of a human immunoglobulin of the subclass IgG1 are used.

Test Method

Assay for the Measurement of the Adhesion of U937 Cells (ATCC CRL 1593) to hVCAM-1 (1-3)-IgG

1. Preparation of Human VCAM-1(1-3)-IgG and Human CD4-IgG

A genetic construct for the expression of the extracellular domains of human VCAM-1 was employed, associated with the genetic sequence of the heavy chain of human immunoglobulin IgG1 (hinge, CH2 and CH3 regions), from Dr. Brian Seed, Massachusetts General Hospital, Boston, USA. The soluble fusion protein hVCAM-1(1-3)-IgG contained the three amino-terminal extracellular immunoglobulin-like domains of human VCAM-1 (Damle and Aruffo, Proc. Natl. Acad. Sci. USA 1991, 88, 6403). CD4-IgG (Zettlmeissl et al, DNA and Cell Biology 1990, 9, 347) served as a fusion protein for negative controls. The recombinant proteins were expressed as soluble proteins after DEAE/dextran-mediated DNA-transfection in COS cells (ATCC CRL1651) according to standard procedures (Ausubel et al., Current protocols in molecular biology, John Wiley & Sons, Inc., 1994).

2. Assay for Measurement of the Adhesion of U937 Cells to hVCAM-1(1-3)-IgG

2.1 96-well microtiter test plates (Nunc Maxisorb) were incubated at room temperature for 1 hour with 100 μl/well of a goat-anti-human IgG antibody solution (10 μg/ml in 50 mM tris, pH 9.5). After removing the antibody solution, washing was carried out once with PBS.

2.2 150 μl/well of a blocking buffer (1% BSA in PBS) were incubated on the plates at room temperature for 0.5 hours. After removing the blocking buffer, washing was carried out once with PBS.

2.3 100 μl per well of a cell culture supernatant of transfected COS cells were incubated on the plates at room temperature for 1.5 hours. The COS cells were transfected with a plasmid which codes for the three N-terminal immunoglobulin-like domains of VCAM-1, coupled to the Fc part of human IgG1 (hVCAM-1(1-3)-IgG). The content of hVCAM-1 (1-3)-IgG was about 0.5-1 p.μ/ml. After removing the culture supernatant, washing was carried out once with PBS.

2.4 The plates were incubated at room temperature for 20 minutes with 100 t1/well of Fc receptor blocking buffer (1 mg/ml of γ-globulin, 100 mM NaCl, 100 μM MgCl₂, 100 μM MnCl₂, 100 μM CaCl₂, 1 mg/ml BSA in 50 mM HEPES, pH 7.5). After removing the Fc receptor blocking buffer, washing was carried out once with PBS.

2.5 20 μl of binding buffer (100 mM NaCl, 100 μM MgCl₂, 100 μM MgCl₂, 100 μM CaCl₂, 1 mg/ml BSA in 50 mM HEPES, pH 7.5), were initially introduced, the substances to be tested were added in 10 μl of binding buffer and the mixture was incubated for 20 minutes. As controls, antibodies against VCAM-1 (BBT, No. BBA6) and against VLA-4

(Immunotech, No. 0764) were used.

2.6 U937 cells were incubated in Fc receptor blocking buffer for 20 minutes and then pipetted in at a concentration of 1×10⁶/ml and in an amount of 100 μl per well (final volume 125 μl/well).

2.7 The plates were slowly immersed at an angle of 45° in stop buffer (100 mM NaCl, 100 μM MgCl₂, 100 μM MnCl₂, 100 μM CaCl₂ in 25 mM Tris, pH 7.5) and shaken off. The process was repeated.

2.8 50 μl/well of a dye solution (16.7 μg/ml of Hoechst dye 33258, 4% formaldehyde, 0.5% Triton X-100 in PBS) were then incubated on the plates for 15 minutes.

2.9 The plates were shaken off and slowly immersed at an angle of 45° in stop buffer (100 mM NaCl, 100 μM MgCl₂, 100 μM MnCl₂, 100 μM CaCl₂ in 25 mM Tris, pH 7.5). The process was repeated. Then, with the liquid, measurements were made in a cytofluorimeter (Millipore) (sensitivity: 5; filter: excitation wavelength: 360 nm, emission wavelength: 460 nm).

The intensity of the light emitted by the stained U937 cells is a measure of the number of the U937 cells adhered to the hVCAM-1(1-3)-IgG and remaining on the plate and thus a measure of the ability of the added test substance to inhibit this adhesion. From the inhibition of the adhesion at various concentrations of the test substance, the concentration IC₅₀ which leads to an inhibition of adhesion by 50% was calculated.

The following test results were obtained:

| | U937/VCAM-1 cell |
|---|---|
| | adhesion test |
| Example | IC₅₀ (μM) |
| 1 | 140 |
| 2 | 15 |
| 4 | 1.1 |
| 5 | 15 |
| 6 | 65 |
| 7 | 25 |
| 8 | 3.5 |
| 9 | 0.5 |
| 10 | 47 |
| 11 | 62 |
| 12 | 2.7 |
| 13 | 3.7 |
| 14 | 0.25 |
| 15 | 32 |
| 16 | 30 |
| 17 | 79 |
| 18 | 0.09 |
| 19 | 0.2 |
| 20 | 2.0 |
| 25 | 22 |
| 33 | 45 |
| 34 | 175 |
| 35 | 250 |
| 36 | 250 |
| 37 | 200 |
| 38 | 45 |
| 39 | 8 |
| 40 | 27 |
| 41 | 0.28 |
| 46 | 6.8 |
| 57 | 17.5 |
| 58 | 25 |
| 59 | 27.5 |
| 62 | 0.37 |
| 63 | 0.22 |
| 67 | 2.25 |
| 69 | 4.5 |
| 70 | 3 |
| 71 | 3.25 |
| 73 | 125 |

It will be apparent to those skilled in the art that various modifications and variations can be made to the compositions and processes of this invention. Thus, it is intended that the present invention cover such modifications and variations, provided they come within the scope of the appended claims and their equivalents.

The disclosure of all publications cited above are expressly incorporated herein by reference in their entireties to the same extent as if each were incorporated by reference individually. The disclosure of German Patent Application No. 19647380.2, for which benefit under 35 USC § 119 is claimed, is expressly incorporated herein in its entirety.

1-28. (canceled)

29. A compound of the formula Id 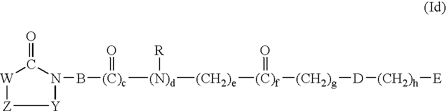
wherein
W is R¹-A-C(R¹³) or R¹-A-CH═C;
Y is a carbonyl, thiocarbonyl, or methylene group;
Z is N(R⁰);
A is a bivalent radical from the group consisting of (C₁-C₆)-alkylene, (C₃-C₇)-cycloalkylene, phenylene, phenylene-(C₁-C₆)-alkyl, (C₁-C₆)-alkylenephenyl, and phenylene-(C₂-C₆)-alkenyl, or a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;
B is a bivalent (C₁-C₆)-alkylene radical which is substituted by a radical from the group consisting of (C₁-C₈)-alkyl, (C₂-C₈)-alkenyl, (C₂-C₈)-alkynyl, (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl and heteroaryl-(C₁-C₆)-alkyl optionally substituted in the heteroaryl radical;
D is C(R²)(R³), N(R³), or CH═C(R³);
E is tetrazolyl, (R⁸O)₂P(O), HOS(O)₂, R⁹NHS(O)₂, or R¹⁰CO;
R is hydrogen, (C₁-C₈)-alkyl, (C₃-C₈)-cycloalkyl, optionally substituted (C₆-C₁₄)-aryl, or (C₆-C₁₄)-aryl-(C₁-C₈)-alkyl optionally substituted in the aryl radical;
R⁰ is hydrogen, (C₁-C₈)-alkyl, (C₃-C₁₂)-cycloalkyl, (C₃-C₁₂)-cycloalkyl-(C₁-C₈)-alkyl, (C₆-C₁₂)-bicycloalkyl, (C₆-C₁₂)-bicycloalkyl-(C₁-C₈)-alkyl, (C₆-C₁₂)-tricycloalkyl, (C₆-C₁₂)-tricycloalkyl-(C₁-C₈)-alkyl, optionally substituted (C₆-C₁₄)-aryl, (C₆-C₁₄)-aryl-(C₁-C₈)-alkyl optionally substituted in the aryl radical, optionally substituted heteroaryl, heteroaryl-(C₁-C₈)-alkyl optionally substituted in the heteroaryl radical, CHO, (C₁-C₈)-alkyl-CO, (C₃-C₁₂)-cycloalkyl-CO, (C₃-C₁₂)-cycloalkyl-(C₁-C₈)-alkyl-CO, (C₆-C₁₂)-bicycloalkyl-CO, (C₆-C₁₂)-bicycloalkyl-(C₁-C₈)-alkyl-CO, (C₆-C₁₂)-tricycloalkyl-CO, (C₆-C₁₂)-tricycloalkyl-(C₁-C₈)-alkyl-CO, optionally substituted (C₆-C₁₄)-aryl-CO, (C₆-C₁₄)-aryl-(C₁-C₈)-alkyl-CO optionally substituted in the aryl radical, optionally substituted heteroaryl-CO, heteroaryl-(C₁-C₈)-alkyl-CO optionally substituted in the heteroaryl radical, (C₁-C₈)-alkyl-S(O)ₙ, (C₃-C₁₂)-cycloalkyl-S(O)ₙ, (C₃-C₁₂)-cycloalkyl-(C₁-C₈)-alkyl-S(O)ₙ, (C₆-C₁₂)-bicycloalkyl-S(O)ₙ, (C₆-C₁₂)-bicycloalkyl-(C₁-C₈)-alkyl-S(O)ₙ, (C₆-C₁₂)-tricycloalkyl-S(O)ₙ, (C₆-C₁₂)-tricycloalkyl-(C₁-C₈)-alkyl-S(O)ₙ, optionally substituted (C₆-C₁₄)-aryl-S(O)ₙ, (C₆-C₁₄)-aryl-(C₁-C₈)-alkyl-S(O), optionally substituted in the aryl radical, optionally substituted heteroaryl-S(O)ₙ or heteroaryl-(C₁-C₈)-alkyl-S(O), optionally substituted in the heteroaryl radical, wherein n is 1 or 2;
R¹ is X—NH—C(═NH)—(CH₂)ₚ or X¹—NH—(CH₂)ₚ, wherein p is 0, 1, 21 or 3;
X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, (R⁸O)₂P(O), cyano, hydroxyl, (C₁-C₆)-alkoxy, (C₆-C₁₄)-aryl-(C₁-C₆)-alkoxy which can also be substituted in the aryl radical, or amino;
X¹ has one of the meanings of X or is R′—NH—C(═N—R″), wherein R′ and R″, independently of one another, have the meanings of X;
R² is hydrogen, (C₁-C₈)-alkyl, optionally substituted (C₆-C₁₄)-aryl, (C₆-C₁₄)-aryl-(C₁-C₈)-alkyl optionally substituted in the aryl radical or (C₃-C₈)-cycloalkyl;
R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, R⁴CO, COOR⁴, CON(CH₃)R¹⁴, CONHR¹¹⁴, CSNHR¹⁴, COOR¹⁵, CON(CH₃)R¹⁵ or CONHR¹⁵;
R⁴ is hydrogen or (C₁-C₂₈)-alkyl which can optionally be mono- or polysubstituted by identical or different radicals R⁴¹;
R^{4′} is hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, halogen, nitro, trifluoromethyl, or the radical R⁵;
R⁵ is optionally substituted (C₆-C₁₄)-aryl, —(C₆-C₁₄)-aryl-(C₁-C₈)-alkyl optionally substituted in the aryl radical, a mono- or bicyclic 5- to 12-membered heterocyclic ring which can be aromatic, partially hydrogenated, or completely hydrogenated, and which can contain one, two, or three identical or different heteroatoms from the group consisting of nitrogen, oxygen, and sulfur, a radical R⁶ or a radical R⁶CO—, wherein the aryl radical and, independently thereof, the heterocyclic radical can be mono- or polysubstituted by identical or different radicals from the group consisting of (C₁-C₁₈)-alkyl, (C₁-C₁₈)-alkoxy, halogen, nitro, amino, and trifluoromethyl;
R⁶ is R⁷R⁸N, R⁷O or R⁷S, or an amino acid side chain, a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or wherein the peptide bond can be reduced to —NH—CH₂—, and their esters and amides, wherein hydrogen or hydroxymethyl can optionally stand in place of free functional groups and/or where free functional groups can be protected by protective groups customary in peptide chemistry;
R⁷ is hydrogen, (C₁-C₁₈)-alkyl, (C₆-C₁₄)-aryl-(C₁-C₈)-alkyl, (C₁-C₁₈)-alkylcarbonyl, (C₁-C₁₈)-alkoxycarbonyl, (C₆-C₁₄)-arylcarbonyl, (C₆-C₁₄)-aryl-(C₁-C₈)-alkylcarbonyl or (C₆-C₁₄)-aryl-(C₁-C₁₈)-alkyloxycarbonyl, wherein the alkyl groups can optionally be substituted by an amino group and/or where the aryl radicals can be mono- or polysubstituted by identical or different radicals from the group consisting of (C₁-C₈)-alkyl, (C₁-C₈)-alkoxy, halogen, nitro, amino and trifluoromethyl, or is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical and/or wherein the peptide bond can be reduced to —NH—CH₂—;
R⁸ is hydrogen, (C₁-C₁₈)-alkyl, optionally substituted (C₆-C₁₄)-aryl or (C₆-C₁₄)-aryl-(C₁-C₈)-alkyl which can also be substituted in the aryl radical;
R⁹ is hydrogen, aminocarbonyl, (C₁-C₁₈)-alkylaminocarbonyl, (C₃-C₈)-cycloalkylaminocarbonyl, optionally substituted (C₆-C₁₄)-arylaminocarbonyl, (C₁-C₁₈)-alkyl, optionally substituted (C₆-C₁₄)-aryl or (C₃-C₈)-cycloalkyl;
R¹⁰ is hydroxyl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;
R¹¹ is hydrogen, (C₁-C₁₈)-alkyl, R¹²CO, optionally substituted (C₆-C₁₄)-aryl-S(O)₂, (C₁-C₁₈)-alkyl-S(O)₂, (C₆-C₁₄)-aryl-(C₁-C₈)-alkyl optionally substituted in the aryl radical or R⁹NHS(O)₂;
R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical, optionally substituted (C₆-C₁₄)-aryloxy, amino or mono- or di-((C₁-C₁₈)-alkyl)amino;
R¹³ is hydrogen, (C₁-C₆)-alkyl, (C₆-C₁₄)-aryl-(C₁-C₈)-alkyl optionally substituted in the aryl radical or (C₃-C₈)-cycloalkyl;
R¹⁴ is hydrogen or (C₁-C₂₈)-alkyl which can optionally be mono- or polysubstituted by identical or different radicals from the group consisting of hydroxyl, hydroxycarbonyl, aminocarbonyl, mono- or di-((C₁-C₁₈)-alkyl)aminocarbonyl, amino-(C₂-C₁₈)-alkylaminocarbonyl, amino-(C₁-C₃)-alkylphenyl-(C₁-C₃)-alkylaminocarbonyl, (C₁-C₁₈)-alkylcarbonylamino-(C₁-C₃)-alkylphenyl-(C₁-C₃)-alkylaminocarbonyl, (C₁-C₁₈)-alkylcarbonylamino-(C₂-C₁₈)-alkylaminocarbonyl, (C₆-C₁₄)-aryl-(C₁-C₈)-alkoxycarbonyl which can also be substituted in the aryl radical, amino, mercapto, (C₁-C₁₈)-alkoxy, (C₁-C₁₈)-alkoxycarbonyl, optionally substituted (C₃-C₈)-cycloalkyl, HOS(O)₂—(C₁-C₃)-alkyl, R⁹NHS(O)₂—(C₁-C₃)-alkyl, (R⁸O)₂P(O)—(C₁-C₃)-alkyl, tetrazolyl-(C₁-C₃)-alkyl, halogen, nitro, trifluoromethyl and R⁵;
R¹⁵ is R¹⁶—(C₁-C₆)-alkyl or R¹⁶;
R⁶ is a 6- to 24-membered bicyclic or tricyclic radical which is saturated or partially unsaturated and which can also contain one to four identical or different heteroatoms from the group consisting of nitrogen, oxygen, and sulfur and which can also be substituted by one or more identical or different substituents from the group consisting of (C₁-C₄)-alkyl and oxo;
c, d, and f; independently of one another, are 0 or 1, but cannot all simultaneously be 0;
e, g, and h, independently of one another, are 0, 1, 2, 3, 4, 5, or 6;
in all its stereoisomeric forms and mixtures thereof in any ratio, and/or its physiologically tolerable salts.

30. The compound of claim 29, wherein
W is R¹-A-C(R¹³);
Y is a carbonyl group;
A is a bivalent radical from the group consisting of (C₃-C₇)-cycloalkylene, phenylene, phenylene-(C₁-C₆)-alkyl, (C₁-C₆)-alkylenephenyl and a bivalent radical of a 5- or 6-membered saturated or unsaturated ring which can contain 1 or 2 nitrogen atoms and can be mono- or disubstituted by (C₁-C₆)-alkyl or doubly bonded oxygen or sulfur;
B is a bivalent methylene radical or ethylene radical which is substituted by a radical from the group consisting of (C₁-C₈)-alkyl, (C₂-C₈)-alkenyl, (C₂-C₈)-alkynyl, (C₃-C₁₀)-cycloalkyl, (C₃-C₁₀)-cycloalkyl-(C₁-C₆)-alkyl, optionally substituted (C₆-C₁₄)-aryl, (C₆-C₁₄)-aryl-(C₁-C₆)-alkyl optionally substituted in the aryl radical, optionally substituted heteroaryl, and heteroaryl-(C₁-C₆)-alkyl optionally substituted in the heteroaryl radical;
D is C(R²)(R³);
E is tetrazolyl or R¹⁰CO;
R is hydrogen or (C₁-C₈)-alkyl;
X is hydrogen, (C₁-C₆)-alkyl, (C₁-C₆)-alkylcarbonyl, (C₁-C₆)-alkoxycarbonyl, (C₁-C₁₈)-alkylcarbonyloxy-(C₁-C₆)-alkoxycarbonyl, optionally substituted (C₆-C₁₄)-arylcarbonyl, optionally substituted (C₆-C₁₄)-aryloxycarbonyl, (C₆-C₁₄)-aryl-(C₁-C₆)-alkoxycarbonyl which can also be substituted in the aryl radical, cyano, hydroxyl, (C₁-C₆)-alkoxy, (C₆-C₁₄)-aryl-(C₁-C₆)-alkoxy which can also be substituted in the aryl radical, or amino;
R² is hydrogen or (C₁-C₈)-alkyl;
R³ is hydrogen, (C₁-C₈)-alkyl, optionally substituted (C₆-C₁₄)-aryl, (C₆-C₁₄)-aryl-(C₁-C₈)-alkyl optionally substituted in the aryl radical, (C₃-C₈)-cycloalkyl, (C₂-C₈)-alkenyl, (C₂-C₈)-alkynyl, (C₂-C₈)-alkenylcarbonyl, (C₂-C₈)-alkynylcarbonyl, pyridyl, R¹¹NH, CON(CH₃)R¹⁴, CONHR¹⁴, CON(CH₃)R¹⁵, or CONHR¹⁵;
R⁵ is optionally substituted (C₆-C₁₄)-aryl, (C₆-C₁₄)-aryl-(C₁-C₈)-alkyl optionally substituted in the aryl radical, a mono- or bicyclic 5- to 12-membered heterocyclic ring which can be aromatic, partially hydrogenated, or completely hydrogenated and which can contain one, two, or three identical or different heteroatoms from the group consisting of nitrogen, oxygen and sulfur, or a radical R⁶CO—, wherein the aryl radical and, independently thereof, the heterocyclic radical, can be mono- or polysubstituted by identical or different radicals from the group consisting of (C₁-C₈)-alkyl, (C₁-C₈)-alkoxy, halogen, nitro, amino, or trifluoromethyl;
R⁶ is a natural or unnatural amino acid, imino acid, optionally N—(C₁-C₈)-alkylated or N—((C₆-C₁₄)-aryl-(C₁-C₈)-alkylated) azamino acid or a dipeptide radical which can also be substituted in the aryl radical, and their esters and amides, wherein free functional groups can be protected by protective groups customary in peptide chemistry;
R¹¹ is R¹²CO, optionally substituted (C₆-C₁₄)-aryl-S(O)₂ or (C₁-C₁₈)-alkyl-S(O)₂;
R¹² is hydrogen, (C₁-C₁₈)-alkyl, (C₂-C₈)-alkenyl, (C₂-C₈)-alkynyl, optionally substituted (C₆-C₁₄)-aryl, (C₁-C₁₈)-alkoxy, (C₆-C₁₄)-aryl-(C₁-C₈)-alkoxy which can also be substituted in the aryl radical or optionally substituted (C₆-C₁₄)-aryloxy;
R¹³ is hydrogen or (C₁-C₄)-alkyl;
R¹⁴ is (C₁-C₁₀)-alkyl which can optionally be mono- or polysubstituted by identical or different radicals from the group consisting of hydroxyl, hydroxycarbonyl, aminocarbonyl, mono- or di-((C₁-C₁₈)-alkyl)amino-carbonyl, (C₆-C₁₄)-aryl-(C₁-C₈)-alkoxycarbonyl which can also be substituted in the aryl radical, (C₁-C₈)-alkoxy, (C₁-C₈)-alkoxycarbonyl, optionally substituted (C₃-C₈)-cycloalkyl, tetrazolyl-(C₁-C₃)-alkyl, trifluoromethyl and R⁵;
c and d are 1, and f is 0;
e and h, independently of one another, are 0 or 1, and g is 0.

31. The compound of claim 29, wherein
B is substituted by a (C₁-C₈)-alkyl radical.

32. A pharmaceutical composition comprising or more compounds of claim 29 and a pharmaceutically acceptable carrier.

33-38. (canceled)

39. The compound of claim 29, wherein
E is R¹⁰CO; and
R⁰ is (C₆-C₁₄)-aryl-(C₁-C₄)-alkyl optionally substituted in the aryl radical.

40. A method of treating a disease or disorder involving inflammation, comprising administering to a subject in need thereof an effective amount of the pharmaceutical composition of claim 32.

41. A method of antagonizing VLA-4, comprising administering to a subject in need thereof an effective amount of the pharmaceutical composition of claim 32.

42. A method of treating a disease or disorder selected from the group consisting of rheumatoid arthritis, inflammatory bowel disease, systemic lupus erythematosus, inflammatory disorders of the central nervous system, asthma, allergies, cardiovascular disorders, arteriosclerosis, multiple sclerosis, restenoses, diabetes, damage to organ transplants, tumor growth, tumor metastasis, melanoma, lymphoma, and malaria, comprising administering to a subject in need thereof an effective amount of the pharmaceutical composition of claim 32.

43. A method of treating a disease or disorder, wherein said disease or disorder exhibits an abnormally large amount of leucocyte adhesion and/or migration, comprising administering to a subject in need thereof an effective amount of the pharmaceutical composition of claim 32.
